SEQUENCE LISTING (12 sequences)

SEQ ID NO: 1 - DNA, 64 bp, synthetic construct
  source 1..64: mol_type = other DNA
  modified_base 8: mod_base = OTHER; note = 3,4-(methylenedioxy)-6-nitrophenylethoxycarbonyl-su bstituted deoxyuridine
  modified_base 15: mod_base = OTHER; note = 3,4-(methylenedioxy)-6-nitrophenylethoxycarbonyl-su bstituted deoxyuridine
  modified_base 22: mod_base = OTHER; note = 3,4-(methylenedioxy)-6-nitrophenylethoxycarbonyl-su bstituted deoxyuridine
  modified_base 29: mod_base = OTHER; note = 3,4-(methylenedioxy)-6-nitrophenylethoxycarbonyl-su bstituted deoxyuridine
  modified_base 36: mod_base = OTHER; note = 3,4-(methylenedioxy)-6-nitrophenylethoxycarbonyl-su bstituted deoxyuridine
  modified_base 43: mod_base = OTHER; note = 3,4-(methylenedioxy)-6-nitrophenylethoxycarbonyl-su bstituted deoxyuridine
  modified_base 50: mod_base = OTHER; note = 3,4-(methylenedioxy)-6-nitrophenylethoxycarbonyl-su bstituted deoxyuridine
  modified_base 57: mod_base = OTHER; note = 3,4-(methylenedioxy)-6-nitrophenylethoxycarbonyl-su bstituted deoxyuridine
caaaaaanaaaaaanaaaaaanaaaaaanaaaaaanaaaaaanaaaaaanaaaaaanaaaaaac

SEQ ID NO: 2 - DNA, 57 bp, synthetic construct
  source 1..57: mol_type = other DNA
gttttttattttttattttttattttttattttttattttttattttttattttttg

SEQ ID NO: 3 - DNA, 21 bp, synthetic construct
  source 1..21: mol_type = other DNA
aaaaaataaaaaaccaaaaaa

SEQ ID NO: 4 - DNA, 68 bp, synthetic construct
  source 1..68: mol_type = other DNA
  modified_base 10: mod_base = OTHER; note = O6-ortho-nitrobenzylguanine
  modified_base 17: mod_base = OTHER; note = O6-ortho-nitrobenzylguanine
  modified_base 24: mod_base = OTHER; note = O6-ortho-nitrobenzylguanine
  modified_base 31: mod_base = OTHER; note = O6-ortho-nitrobenzylguanine
  modified_base 38: mod_base = OTHER; note = O6-ortho-nitrobenzylguanine
  modified_base 45: mod_base = OTHER; note = O6-ortho-nitrobenzylguanine
  modified_base 52: mod_base = OTHER; note = O6-ortho-nitrobenzylguanine
  modified_base 59: mod_base = OTHER; note = O6-ortho-nitrobenzylguanine
cctaaaaaanaaaaaanaaaaaanaaaaaanaaaaaanaaaaaanaaaaaanaaaaaanaaaaaacga

SEQ ID NO: 5 - DNA, 68 bp, synthetic construct; identical to SEQ ID NO: 4
  source 1..68: mol_type = other DNA
  modified_base 1: mod_base = OTHER; note = 5'-phosphate modified nucleotide
  modified_base 10: mod_base = OTHER; note = O6-ortho-nitrobenzylguanine
  modified_base 17: mod_base = OTHER; note = O6-ortho-nitrobenzylguanine
  modified_base 24: mod_base = OTHER; note = O6-ortho-nitrobenzylguanine
  modified_base 31: mod_base = OTHER; note = O6-ortho-nitrobenzylguanine
  modified_base 38: mod_base = OTHER; note = O6-ortho-nitrobenzylguanine
  modified_base 45: mod_base = OTHER; note = O6-ortho-nitrobenzylguanine
  modified_base 52: mod_base = OTHER; note = O6-ortho-nitrobenzylguanine
  modified_base 59: mod_base = OTHER; note = O6-ortho-nitrobenzylguanine

SEQ ID NO: 6 - DNA, 18 bp, synthetic construct
  source 1..18: mol_type = other DNA
ttttttaggtcgtttttt

SEQ ID NO: 7 - DNA, 68 bp, synthetic construct
  source 1..68: mol_type = other DNA
  modified_base 1: mod_base = OTHER; note = 5'-phosphate modified nucleotide
gttttttcttttttcttttttcttttttcttttttcttttttcttttttcttttttcttttttaggtc

SEQ ID NO: 8 - DNA, 143 bp, synthetic construct
  source 1..143: mol_type = other DNA
  modified_base 7: mod_base = OTHER; note = O6-nitrobenzylguanine
  modified_base 9: mod_base = OTHER; note = N6-coumarinylmethyl-adenine
  modified_base 20: mod_base = OTHER; note = O6-nitrobenzylguanine
  modified_base 22: mod_base = OTHER; note = N6-coumarinylmethyl-adenine
  modified_base 33: mod_base = OTHER; note = O6-nitrobenzylguanine
  modified_base 35: mod_base = OTHER; note = N6-coumarinylmethyl-adenine
  modified_base 46: mod_base = OTHER; note = O6-nitrobenzylguanine
  modified_base 48: mod_base = OTHER; note = N6-coumarinylmethyl-adenine
  modified_base 59: mod_base = OTHER; note = O6-nitrobenzylguanine
  modified_base 61: mod_base = OTHER; note = N6-coumarinylmethyl-adenine
  modified_base 72: mod_base = OTHER; note = O6-nitrobenzylguanine
  modified_base 74: mod_base = OTHER; note = N6-coumarinylmethyl-adenine
  modified_base 85: mod_base = OTHER; note = O6-nitrobenzylguanine
  modified_base 87: mod_base = OTHER; note = N6-coumarinylmethyl-adenine
  modified_base 98: mod_base = OTHER; note = O6-nitrobenzylguanine
  modified_base 100: mod_base = OTHER; note = N6-coumarinylmethyl-adenine
  modified_base 111: mod_base = OTHER; note = O6-nitrobenzylguanine
  modified_base 113: mod_base = OTHER; note = N6-coumarinylmethyl-adenine
  modified_base 124: mod_base = OTHER; note = O6-nitrobenzylguanine
  modified_base 126: mod_base = OTHER; note = N6-coumarinylmethyl-adenine
  modified_base 137: mod_base = OTHER; note = O6-nitrobenzylguanine
  modified_base 139: mod_base = OTHER; note = N6-coumarinylmethyl-adenine
tcgattnanaattattcctnanaattattcctnanaattattcctnanaattattcctnanaattattcctnanaattattcctnanaattattcctnanaattattcctnanaattattcctnantttatcttatnantcga

SEQ ID NO: 9 - DNA, 143 bp, synthetic construct
  source 1..143: mol_type = other DNA
tcgattcataagataaattcaggaataattttcaggaataattttcaggaataattttcaggaataattttcaggaataattttcaggaataattttcaggaataattttcaggaataattttcaggaataattttcaatcga

SEQ ID NO: 10 - DNA, 143 bp, synthetic construct
  source 1..143: mol_type = other DNA
  modified_base 7: mod_base = OTHER; note = O6-nitrobenzylguanine
  modified_base 46: mod_base = OTHER; note = O6-nitrobenzylguanine
  modified_base 59: mod_base = OTHER; note = O6-nitrobenzylguanine
  modified_base 85: mod_base = OTHER; note = O6-nitrobenzylguanine
  modified_base 124: mod_base = OTHER; note = O6-nitrobenzylguanine
  modified_base 137: mod_base = OTHER; note = O6-nitrobenzylguanine
tcgattnaaaattattcctgaaaattattcctgaaaattattcctnaaaattattcctnaaaattattcctgaaaattattcctnaaaattattcctgaaaattattcctgaaaattattcctnaatttatcttatnaatcga

SEQ ID NO: 11 - DNA, 42 bp, synthetic construct
  source 1..42: mol_type = other DNA
  misc_difference 1: note = Convertable base may be guanine, O6-guanine, O6-thioguanine, N2-guanine, N7-guanine, N6-adenine, N5-adenine, O4-thymine, O4-uracil, N3-thymine, 2-thio-thymine, 4-thio-thymine, N4-cytosine, N3-cytosine, 4,5-dimethoxy-2-nitrobenzyl carbamate, (2-nitrophenyl)methanol, Phenylmethanol, 3-(2,4-dimethyl-3 ,6-dioxocyclohexa-1,4-dien-1-yl)-3-methylbutanoic acid, or (6-(dimethylamino)-2-oxo-2H-chromen-4-yl)methyl carbamate
  misc_difference 7: note = Convertable base may be thymine, O6-guanine, O6-thioguanine, N2-guanine, N7-guanine, N6-adenine, N5-adenine, O4-thymine, O4-uracil, N3-thymine, 2-thio-thymine, 4-thio-thymine, N4-cytosine, N3-cytosine, 4,5-dimethoxy-2-nitrobenzyl carbamate, (2-nitrophenyl)methanol, Phenylmethanol, 3-(2,4-dimethyl-3 ,6-dioxocyclohexa-1,4-dien-1-yl)-3-methylbutanoic acid, or (6-(dimethylamino)-2-oxo-2H-chromen-4-yl)methyl carbamate
  misc_difference 9: note = Convertable base may be guanine, O6-guanine, O6-thioguanine, N2-guanine, N7-guanine, N6-adenine, N5-adenine, O4-thymine, O4-uracil, N3-thymine, 2-thio-thymine, 4-thio-thymine, N4-cytosine, N3-cytosine, 4,5-dimethoxy-2-nitrobenzyl carbamate, (2-nitrophenyl)methanol, Phenylmethanol, 3-(2,4-dimethyl-3 ,6-dioxocyclohexa-1,4-dien-1-yl)-3-methylbutanoic acid, or (6-(dimethylamino)-2-oxo-2H-chromen-4-yl)methyl carbamate
  misc_difference 12: note = Convertable base may be thymine, O6-guanine, O6-thioguanine, N2-guanine, N7-guanine, N6-adenine, N5-adenine, O4-thymine, O4-uracil, N3-thymine, 2-thio-thymine, 4-thio-thymine, N4-cytosine, N3-cytosine, 4,5-dimethoxy-2-nitrobenzyl carbamate, (2-nitrophenyl)methanol, Phenylmethanol, 3-(2,4-dimethyl-3 ,6-dioxocyclohexa-1,4-dien-1-yl)-3-methylbutanoic acid, or (6-(dimethylamino)-2-oxo-2H-chromen-4-yl)methyl carbamate
  misc_difference 13: note = Convertable base may be guanine, O6-guanine, O6-thioguanine, N2-guanine, N7-guanine, N6-adenine, N5-adenine, O4-thymine, O4-uracil, N3-thymine, 2-thio-thymine, 4-thio-thymine, N4-cytosine, N3-cytosine, 4,5-dimethoxy-2-nitrobenzyl carbamate, (2-nitrophenyl)methanol, Phenylmethanol, 3-(2,4-dimethyl-3 ,6-dioxocyclohexa-1,4-dien-1-yl)-3-methylbutanoic acid, or (6-(dimethylamino)-2-oxo-2H-chromen-4-yl)methyl carbamate
  misc_difference 17: note = Convertable base may be thymine, O6-guanine, O6-thioguanine, N2-guanine, N7-guanine, N6-adenine, N5-adenine, O4-thymine, O4-uracil, N3-thymine, 2-thio-thymine, 4-thio-thymine, N4-cytosine, N3-cytosine, 4,5-dimethoxy-2-nitrobenzyl carbamate, (2-nitrophenyl)methanol, Phenylmethanol, 3-(2,4-dimethyl-3 ,6-dioxocyclohexa-1,4-dien-1-yl)-3-methylbutanoic acid, or (6-(dimethylamino)-2-oxo-2H-chromen-4-yl)methyl carbamate
  misc_difference 18: note = Convertable base may be guanine, O6-guanine, O6-thioguanine, N2-guanine, N7-guanine, N6-adenine, N5-adenine, O4-thymine, O4-uracil, N3-thymine, 2-thio-thymine, 4-thio-thymine, N4-cytosine, N3-cytosine, 4,5-dimethoxy-2-nitrobenzyl carbamate, (2-nitrophenyl)methanol, Phenylmethanol, 3-(2,4-dimethyl-3 ,6-dioxocyclohexa-1,4-dien-1-yl)-3-methylbutanoic acid, or (6-(dimethylamino)-2-oxo-2H-chromen-4-yl)methyl carbamate
  misc_difference 21: note = Convertable base may be thymine, O6-guanine, O6-thioguanine, N2-guanine, N7-guanine, N6-adenine, N5-adenine, O4-thymine, O4-uracil, N3-thymine, 2-thio-thymine, 4-thio-thymine, N4-cytosine, N3-cytosine, 4,5-dimethoxy-2-nitrobenzyl carbamate, (2-nitrophenyl)methanol, Phenylmethanol, 3-(2,4-dimethyl-3 ,6-dioxocyclohexa-1,4-dien-1-yl)-3-methylbutanoic acid, or (6-(dimethylamino)-2-oxo-2H-chromen-4-yl)methyl carbamate
  misc_difference 22: note = Convertable base may be guanine, O6-guanine, O6-thioguanine, N2-guanine, N7-guanine, N6-adenine, N5-adenine, O4-thymine, O4-uracil, N3-thymine, 2-thio-thymine, 4-thio-thymine, N4-cytosine, N3-cytosine, 4,5-dimethoxy-2-nitrobenzyl carbamate, (2-nitrophenyl)methanol, Phenylmethanol, 3-(2,4-dimethyl-3 ,6-dioxocyclohexa-1,4-dien-1-yl)-3-methylbutanoic acid, or (6-(dimethylamino)-2-oxo-2H-chromen-4-yl)methyl carbamate
  misc_difference 25: note = Convertable base may be thymine, O6-guanine, O6-thioguanine, N2-guanine, N7-guanine, N6-adenine, N5-adenine, O4-thymine, O4-uracil, N3-thymine, 2-thio-thymine, 4-thio-thymine, N4-cytosine, N3-cytosine, 4,5-dimethoxy-2-nitrobenzyl carbamate, (2-nitrophenyl)methanol, Phenylmethanol, 3-(2,4-dimethyl-3 ,6-dioxocyclohexa-1,4-dien-1-yl)-3-methylbutanoic acid, or (6-(dimethylamino)-2-oxo-2H-chromen-4-yl)methyl carbamate
  misc_difference 26: note = Convertable base may be guanine, O6-guanine, O6-thioguanine, N2-guanine, N7-guanine, N6-adenine, N5-adenine, O4-thymine, O4-uracil, N3-thymine, 2-thio-thymine, 4-thio-thymine, N4-cytosine, N3-cytosine, 4,5-dimethoxy-2-nitrobenzyl carbamate, (2-nitrophenyl)methanol, Phenylmethanol, 3-(2,4-dimethyl-3 ,6-dioxocyclohexa-1,4-dien-1-yl)-3-methylbutanoic acid, or (6-(dimethylamino)-2-oxo-2H-chromen-4-yl)methyl carbamate
  misc_difference 28: note = Convertable base may be thymine, O6-guanine, O6-thioguanine, N2-guanine, N7-guanine, N6-adenine, N5-adenine, O4-thymine, O4-uracil, N3-thymine, 2-thio-thymine, 4-thio-thymine, N4-cytosine, N3-cytosine, 4,5-dimethoxy-2-nitrobenzyl carbamate, (2-nitrophenyl)methanol, Phenylmethanol, 3-(2,4-dimethyl-3 ,6-dioxocyclohexa-1,4-dien-1-yl)-3-methylbutanoic acid, or (6-(dimethylamino)-2-oxo-2H-chromen-4-yl)methyl carbamate
  misc_difference 30: note = Convertable base may be guanine, O6-guanine, O6-thioguanine, N2-guanine, N7-guanine, N6-adenine, N5-adenine, O4-thymine, O4-uracil, N3-thymine, 2-thio-thymine, 4-thio-thymine, N4-cytosine, N3-cytosine, 4,5-dimethoxy-2-nitrobenzyl carbamate, (2-nitrophenyl)methanol, Phenylmethanol, 3-(2,4-dimethyl-3 ,6-dioxocyclohexa-1,4-dien-1-yl)-3-methylbutanoic acid, or (6-(dimethylamino)-2-oxo-2H-chromen-4-yl)methyl carbamate
  misc_difference 33: note = Convertable base may be thymine, O6-guanine, O6-thioguanine, N2-guanine, N7-guanine, N6-adenine, N5-adenine, O4-thymine, O4-uracil, N3-thymine, 2-thio-thymine, 4-thio-thymine, N4-cytosine, N3-cytosine, 4,5-dimethoxy-2-nitrobenzyl carbamate, (2-nitrophenyl)methanol, Phenylmethanol, 3-(2,4-dimethyl-3 ,6-dioxocyclohexa-1,4-dien-1-yl)-3-methylbutanoic acid, or (6-(dimethylamino)-2-oxo-2H-chromen-4-yl)methyl carbamate
  misc_difference 34: note = Convertable base may be guanine, O6-guanine, O6-thioguanine, N2-guanine, N7-guanine, N6-adenine, N5-adenine, O4-thymine, O4-uracil, N3-thymine, 2-thio-thymine, 4-thio-thymine, N4-cytosine, N3-cytosine, 4,5-dimethoxy-2-nitrobenzyl carbamate, (2-nitrophenyl)methanol, Phenylmethanol, 3-(2,4-dimethyl-3 ,6-dioxocyclohexa-1,4-dien-1-yl)-3-methylbutanoic acid, or (6-(dimethylamino)-2-oxo-2H-chromen-4-yl)methyl carbamate
  misc_difference 36: note = Convertable base may be thymine, O6-guanine, O6-thioguanine, N2-guanine, N7-guanine, N6-adenine, N5-adenine, O4-thymine, O4-uracil, N3-thymine, 2-thio-thymine, 4-thio-thymine, N4-cytosine, N3-cytosine, 4,5-dimethoxy-2-nitrobenzyl carbamate, (2-nitrophenyl)methanol, Phenylmethanol, 3-(2,4-dimethyl-3 ,6-dioxocyclohexa-1,4-dien-1-yl)-3-methylbutanoic acid, or (6-(dimethylamino)-2-oxo-2H-chromen-4-yl)methyl carbamate
  misc_difference 39: note = Convertable base may be guanine, O6-guanine, O6-thioguanine, N2-guanine, N7-guanine, N6-adenine, N5-adenine, O4-thymine, O4-uracil, N3-thymine, 2-thio-thymine, 4-thio-thymine, N4-cytosine, N3-cytosine, 4,5-dimethoxy-2-nitrobenzyl carbamate, (2-nitrophenyl)methanol, Phenylmethanol, 3-(2,4-dimethyl-3 ,6-dioxocyclohexa-1,4-dien-1-yl)-3-methylbutanoic acid, or (6-(dimethylamino)-2-oxo-2H-chromen-4-yl)methyl carbamate
  misc_difference 41: note = Convertable base may be thymine, O6-guanine, O6-thioguanine, N2-guanine, N7-guanine, N6-adenine, N5-adenine, O4-thymine, O4-uracil, N3-thymine, 2-thio-thymine, 4-thio-thymine, N4-cytosine, N3-cytosine, 4,5-dimethoxy-2-nitrobenzyl carbamate, (2-nitrophenyl)methanol, Phenylmethanol, 3-(2,4-dimethyl-3 ,6-dioxocyclohexa-1,4-dien-1-yl)-3-methylbutanoic acid, or (6-(dimethylamino)-2-oxo-2H-chromen-4-yl)methyl carbamate
naccatnancannatcnngannatnntnancannancangnc

SEQ ID NO: 12 - DNA, 42 bp, synthetic construct
  source 1..42: mol_type = other DNA
  misc_difference 1: note = Convertable base may be guanine, O6-guanine, O6-thioguanine, N2-guanine, N7-guanine, N6-adenine, N5-adenine, O4-thymine, O4-uracil, N3-thymine, 2-thio-thymine, 4-thio-thymine, N4-cytosine, N3-cytosine, 4,5-dimethoxy-2-nitrobenzyl carbamate, (2-nitrophenyl)methanol, Phenylmethanol, 3-(2,4-dimethyl-3 ,6-dioxocyclohexa-1,4-dien-1-yl)-3-methylbutanoic acid, or (6-(dimethylamino)-2-oxo-2H-chromen-4-yl)methyl carbamate
  misc_difference 12: note = Convertable base may be thymine, O6-guanine, O6-thioguanine, N2-guanine, N7-guanine, N6-adenine, N5-adenine, O4-thymine, O4-uracil, N3-thymine, 2-thio-thymine, 4-thio-thymine, N4-cytosine, N3-cytosine, 4,5-dimethoxy-2-nitrobenzyl carbamate, (2-nitrophenyl)methanol, Phenylmethanol, 3-(2,4-dimethyl-3 ,6-dioxocyclohexa-1,4-dien-1-yl)-3-methylbutanoic acid, or (6-(dimethylamino)-2-oxo-2H-chromen-4-yl)methyl carbamate
  misc_difference 13: note = Convertable base may be guanine, O6-guanine, O6-thioguanine, N2-guanine, N7-guanine, N6-adenine, N5-adenine, O4-thymine, O4-uracil, N3-thymine, 2-thio-thymine, 4-thio-thymine, N4-cytosine, N3-cytosine, 4,5-dimethoxy-2-nitrobenzyl carbamate, (2-nitrophenyl)methanol, Phenylmethanol, 3-(2,4-dimethyl-3 ,6-dioxocyclohexa-1,4-dien-1-yl)-3-methylbutanoic acid, or (6-(dimethylamino)-2-oxo-2H-chromen-4-yl)methyl carbamate
  misc_difference 18: note = Convertable base may be guanine, O6-guanine, O6-thioguanine, N2-guanine, N7-guanine, N6-adenine, N5-adenine, O4-thymine, O4-uracil, N3-thymine, 2-thio-thymine, 4-thio-thymine, N4-cytosine, N3-cytosine, 4,5-dimethoxy-2-nitrobenzyl carbamate, (2-nitrophenyl)methanol, Phenylmethanol, 3-(2,4-dimethyl-3 ,6-dioxocyclohexa-1,4-dien-1-yl)-3-methylbutanoic acid, or (6-(dimethylamino)-2-oxo-2H-chromen-4-yl)methyl carbamate
  misc_difference 21: note = Convertable base may be thymine, O6-guanine, O6-thioguanine, N2-guanine, N7-guanine, N6-adenine, N5-adenine, O4-thymine, O4-uracil, N3-thymine, 2-thio-thymine, 4-thio-thymine, N4-cytosine, N3-cytosine, 4,5-dimethoxy-2-nitrobenzyl carbamate, (2-nitrophenyl)methanol, Phenylmethanol, 3-(2,4-dimethyl-3 ,6-dioxocyclohexa-1,4-dien-1-yl)-3-methylbutanoic acid, or (6-(dimethylamino)-2-oxo-2H-chromen-4-yl)methyl carbamate
  misc_difference 25: note = Convertable base may be thymine, O6-guanine, O6-thioguanine, N2-guanine, N7-guanine, N6-adenine, N5-adenine, O4-thymine, O4-uracil, N3-thymine, 2-thio-thymine, 4-thio-thymine, N4-cytosine, N3-cytosine, 4,5-dimethoxy-2-nitrobenzyl carbamate, (2-nitrophenyl)methanol, Phenylmethanol, 3-(2,4-dimethyl-3 ,6-dioxocyclohexa-1,4-dien-1-yl)-3-methylbutanoic acid, or (6-(dimethylamino)-2-oxo-2H-chromen-4-yl)methyl carbamate
  misc_difference 26: note = Convertable base may be guanine, O6-guanine, O6-thioguanine, N2-guanine, N7-guanine, N6-adenine, N5-adenine, O4-thymine, O4-uracil, N3-thymine, 2-thio-thymine, 4-thio-thymine, N4-cytosine, N3-cytosine, 4,5-dimethoxy-2-nitrobenzyl carbamate, (2-nitrophenyl)methanol, Phenylmethanol, 3-(2,4-dimethyl-3 ,6-dioxocyclohexa-1,4-dien-1-yl)-3-methylbutanoic acid, or (6-(dimethylamino)-2-oxo-2H-chromen-4-yl)methyl carbamate
  misc_difference 28: note = Convertable base may be thymine, O6-guanine, O6-thioguanine, N2-guanine, N7-guanine, N6-adenine, N5-adenine, O4-thymine, O4-uracil, N3-thymine, 2-thio-thymine, 4-thio-thymine, N4-cytosine, N3-cytosine, 4,5-dimethoxy-2-nitrobenzyl carbamate, (2-nitrophenyl)methanol, Phenylmethanol, 3-(2,4-dimethyl-3 ,6-dioxocyclohexa-1,4-dien-1-yl)-3-methylbutanoic acid, or (6-(dimethylamino)-2-oxo-2H-chromen-4-yl)methyl carbamate
  misc_difference 34: note = Convertable base may be guanine, O6-guanine, O6-thioguanine, N2-guanine, N7-guanine, N6-adenine, N5-adenine, O4-thymine, O4-uracil, N3-thymine, 2-thio-thymine, 4-thio-thymine, N4-cytosine, N3-cytosine, 4,5-dimethoxy-2-nitrobenzyl carbamate, (2-nitrophenyl)methanol, Phenylmethanol, 3-(2,4-dimethyl-3 ,6-dioxocyclohexa-1,4-dien-1-yl)-3-methylbutanoic acid, or (6-(dimethylamino)-2-oxo-2H-chromen-4-yl)methyl carbamate
  misc_difference 36: note = Convertable base may be thymine, O6-guanine, O6-thioguanine, N2-guanine, N7-guanine, N6-adenine, N5-adenine, O4-thymine, O4-uracil, N3-thymine, 2-thio-thymine, 4-thio-thymine, N4-cytosine, N3-cytosine, 4,5-dimethoxy-2-nitrobenzyl carbamate, (2-nitrophenyl)methanol, Phenylmethanol, 3-(2,4-dimethyl-3 ,6-dioxocyclohexa-1,4-dien-1-yl)-3-methylbutanoic acid, or (6-(dimethylamino)-2-oxo-2H-chromen-4-yl)methyl carbamate
  misc_difference 41: note = Convertable base may be thymine, O6-guanine, O6-thioguanine, N2-guanine, N7-guanine, N6-adenine, N5-adenine, O4-thymine, O4-uracil, N3-thymine, 2-thio-thymine, 4-thio-thymine, N4-cytosine, N3-cytosine, 4,5-dimethoxy-2-nitrobenzyl carbamate, (2-nitrophenyl)methanol, Phenylmethanol, 3-(2,4-dimethyl-3 ,6-dioxocyclohexa-1,4-dien-1-yl)-3-methylbutanoic acid, or (6-(dimethylamino)-2-oxo-2H-chromen-4-yl)methyl carbamate
naccattagcannatctngangatnntnagcatnancaggnc

(19) United States
(12) Patent Application Publication
(10) Pub. No.: US 2024/0427528 A1
(43) Pub. Date: Dec. 26, 2024

(54) COMPOSITIONS, SYSTEMS, AND METHODS FOR NUCLEIC ACID DATA STORAGE

(71) Applicant: NAIO, Inc., San Carlos, CA (US)

(72) Inventor: Eric Kool, Stanford, CA (US)

(21) Appl. No.: 18/410,087

(22) Filed: Jan. 11, 2024

Related U.S. Application Data
(63) Continuation of application No. PCT/US2022/038591, filed on Jul. 27, 2022.
(60) Provisional application No. 63/226,720, filed on Jul. 28, 2021.
(60) Provisional application No. 63/269,324, filed on Mar. 14, 2022.

(51) Int. Cl. G06F 3/06; G16B 50/30

(52) U.S. Cl. CPC G06F 3/0673; G06F 3/0604; G06F 3/0655; G16B 50/30

(57) ABSTRACT

Provided herein are writable polymers (e.g., writable nucleic acid polymers) for data storage and related methods. Generally, a writable polymer (e.g., a nucleic acid polymer) contains one or more convertible residues (e.g., convertible nucleobases) that are capable of being converted from a first state to a second state, the first and the second state being different. Various methods can be utilized to generate a writable nucleic polymer, such as polymerase extension via rolling circle reaction or chemical synthesis and ligation. Also provided herein are various methods for writing or encoding a writable nucleic acid polymer by selectively converting nucleobases into the second state. Also provided herein are various methods for reading or decoding a nucleic acid polymer encoded with data.

### CROSS REFERENCE TO RELATED APPLICATIONS

This application is a continuation application of International Patent Application No. PCT/US2022/038591, filed Jul. 27, 2022, which claims the benefit of U.S. Provisional Patent Application No. 63/226,720, filed Jul. 28, 2021, and U.S. Provisional Patent Application No. 63/269,324, filed on Mar. 14, 2022, the content of each of which is incorporated by reference herein in its entirety.

### SEQUENCE LISTING

The instant application contains a Sequence Listing which has been submitted electronically in XML format and is hereby incorporated by reference in its entirety. Said XML copy, created on Sep. 4, 2024, is named 63281-702_301_SL.xml and is 64,934 bytes in size.”

### TECHNICAL FIELD

The disclosure is generally directed to compositions, systems, and methods for storing data in nucleic acid molecules.

### BACKGROUND

As the amount of digital data increases, the complications of storing digital data long term is becoming a rapidly growing issue. Electronically or magnetically archived digital data can easily be manipulated, distorted, and/or lost while in storage. While efficient solid-state electronic methods for archival data storage exist, they are not stable over a period of years, resulting in loss of data unless the data is periodically rewritten or transferred to a new device. Similarly, magnetic tape is commonly used for data archiving, but it also degrades over time. Therefore, ways to efficiently encode and store data, especially over long periods, are being pursued very actively.

Nucleic acid molecules (especially DNA) offer a potential solution for overcoming issues with data storage. With its sequences of repeated bases, nucleic acid polymers are essentially biochemical molecules of digital information, which can be stably stored at high densities for extremely long durations in time. Natural DNA contains digital information encoded in the four bases: A, C. T, and G, and can be used to encode binary data in its sequence in synthesized strands. A single polymer of DNA can be very long (such as in chromosomes) and encodes millions of bits of data. It has been estimated that 1 cubic inch of DNA can encode 10¹⁸ bytes of data. Furthermore. DNA is relatively stable, and has yielded sequence information even from samples tens or hundreds of thousands of years old. Thus. DNA offers considerable promise for archiving data.

Further, to facilitate the access to stored data in nucleic acid molecules, the stored data can be read rapidly and cheaply via high-throughput sequencing techniques. Advances in sequencing technology have greatly lowered the cost and increased the speed of sequencing, allowing data in DNA to be read efficiently. Newer long-read single molecule technologies enable rapid reading of bases in single DNA molecules tens of thousands of bases in length. Newer nanopore technologies enable the reading of sequence from single molecules of DNA in seconds to minutes (see N Kono and K. Arakawa. Dev Growth Differ. 2019; 61:316-326; and Q Chen and Z. Liu. Sensors (Basel). 2019; 19:1886; the disclosures of which are each incorporated herein by reference), and can read sequences of strands tens of thousands or base pairs in length or more.

Although nucleic acids are a great potential source of data storage, the process of synthesizing of nucleic acids in particular data-defining sequences is inefficient and thus the process of encoding the nucleic acids is a substantial barrier to utilizing nucleic acids as data storage. Current approaches for storing data in DNA involve chemical or enzymatic synthesis of strands of arbitrary sequences that encode digital information (see G. M. Church. Y. Gao. and S. Kosuri Science. 2012; 337:1628: X. Chengtao, et al., Nucleic Acids Res. 2021; 49:5451-5469; and E. Yoo, et al., Comput Struct Biotechnol J. 2021:19:2468-2476; the disclosures of which are each incorporated herein by reference). Oligonucleotide synthesizers can produce DNAs of length up to roughly 100-200 nucleotides. Specialized synthesizers can produce hundreds or thousands of oligonucleotides at one time, which promises higher throughput of data writing. In addition to chemical DNA synthesis, enzymatic approaches involving polymerases or other enzymes are also under investigation for creating DNAs of arbitrary data-encoding sequence. These involve adding specialized nucleotides one at a time, or short segments of DNA, step by step.

The approach of encoding data in DNA during synthesis is limited by yield, strand length, time, and cost. Current efficient DNA synthesizers produce strands up to roughly 200 nucleotides, and thus encode relatively small amounts of information. Large numbers of different oligonucleotides must be synthesized to compensate for the short sequences. Oligonucleotide synthesis requires excess reagents to achieve high stepwise yields, and requires expensive consumption of reagents and solvents. It also requires time to achieve these high yields for each nucleotide addition (commonly 1-5 min for each step), which implies the need for extended time for encoding larger amounts of data. Common enzymatic approaches under development similarly add nucleotides or groups of nucleotides in stepwise fashion, and have not yet greatly improved on the ability to produce very long strands and encode large amounts of data. Because the enzymatic synthesis approaches also occur stepwise, they also have limits in the speed of data encoding. Further, since both the above chemical and enzymatic strategies typically produce relatively short strands, they may not be ideal for single molecule sequencing, and instead may rely on sequencing methods that require larger amounts of each written DNA.

### SUMMARY OF THE DISCLOSURE

In one aspect, provided herein are polymers for encoding data, comprising:
a plurality of convertible residues iteratively spaced along and covalently linked to the backbone of the polymer,
wherein each of the plurality of convertible residues has a first state and is capable of being converted from the first state into a second state, the first state and the second state being different and the plurality of convertible residues in the first state and the second state are readable by a polymerase enzyme;
wherein the plurality of convertible residues are covalently linked to the polymer in the first state and in the second state.

In certain embodiments, the polymer is a nucleic acid polymer and the plurality of convertible residues are convertible nucleobases.

In certain embodiments, the nucleic acid polymer is a single-stranded nucleic acid polymer.

In certain embodiments, the nucleic acid polymer is double-stranded nucleic acid polymer.

In certain embodiments, the nucleic acid polymer comprises Deoxyribonucleic acid (DNA), Ribonucleic acid (RNA), phosphorothioate DNA, glycerol nucleic acids (GNA), threose nucleic acids (TNA), locked nucleic acids (LNA), or a combination thereof.

In certain embodiments, the nucleic acid polymer comprises greater than 10 convertible residues.

In certain embodiments, the ratio of the total number of nucleotides to the convertible residues in the nucleic acid polymer is between 2 to 100.

In certain embodiments, the plurality of convertible nucleobases are non-naturally occurring nucleobases.

In certain embodiments, the plurality of convertible nucleobases are modified naturally occurring nucleobases or derivatives of naturally occurring nucleobases.

In certain embodiments, each of the plurality of convertible nucleobases comprises a chemically modifiable moiety.

In certain embodiments, each of the plurality of convertible nucleobases the chemically modifiable moiety is directly attached to the base of the convertible nucleobases.

In certain embodiments, each of the plurality of convertible nucleobases the chemically modifiable moiety is attached to the base without a linker or a sidechain.

In certain embodiments, the plurality of convertible nucleobases are covalently linked to the backbone of the nucleic acid via the sugar.

In certain embodiments, the chemically modifiable moiety is activatable by light, voltage, enzymatic agent, chemical reagent, or a redox agent, thereby converting from the first state into the second state.

In certain embodiments, the chemically modifiable moiety is activatable by light, thereby converting from the first state into the second state.

In certain embodiments, the conversion from the first state into the second state occurs via an irreversible reaction.

In certain embodiments, the convertible nucleobase becomes a naturally occurring nucleobase after conversion into the second state.

In certain embodiments, the convertible nucleobase becomes guanine, adenine, thymine, uracil or cytosine after conversion into the second state.

In certain embodiments, the backbone of the polymer (e.g., phosphate and sugar in nucleic acid polymer) remain unchanged during the conversion from the first state into the second state.

In certain embodiments, the polymer comprises two or more different sets of convertible residues, each set of convertible residues has a first state and is capable of being converted from the first state into a second state, the first state and the second state being different.

In certain embodiments, each of the plurality of convertible residues comprises a chemically modifiable moiety that can be activated by light.

In certain embodiments, the two or more different sets of convertible residues are activatable by light of different wavelengths.

In certain embodiments, a first set of convertible residues is activatable by light of a first wavelength, and a second set of convertible residues is activatable by light of a second wavelength, the first wavelength and the second wavelength being different.

In certain embodiments, the chemically modifiable moiety comprises one or more photo-removable groups.

In certain embodiments, the chemically modifiable moiety is a leaving group.

In certain embodiments, the one or more photo-removable groups are:

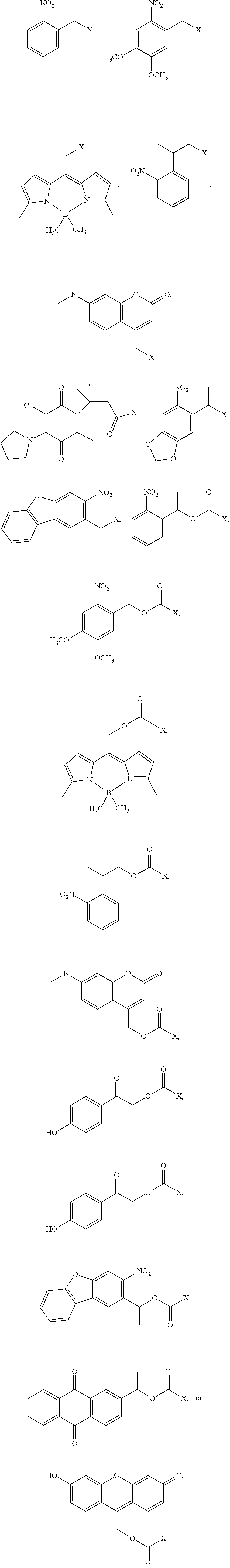

wherein X represents NR2, NHR, OR, or SR, and wherein R is the nucleobase to which the photo-removable group is attached.

In certain embodiments, the plurality of convertible nucleobases are capable of being converted by light of a wavelength of 325 nm, 360 nm, or 400 nm.

In certain embodiments, the plurality of convertible nucleobases are capable of being converted by light of a wavelength of between 400 nm to 850 nm.

In certain embodiments, each of the plurality of convertible nucleobases comprises a chemically modifiable moiety that is activatable by redox.

In certain embodiments, the chemically modifiable moiety is capable of being activated by localized oxidation.

In certain embodiments, the chemically modifiable moiety is capable of being activated by oxidation using electrodes.

In certain embodiments, a nucleotide comprising the convertible nucleobase is selected from the group consisting of:

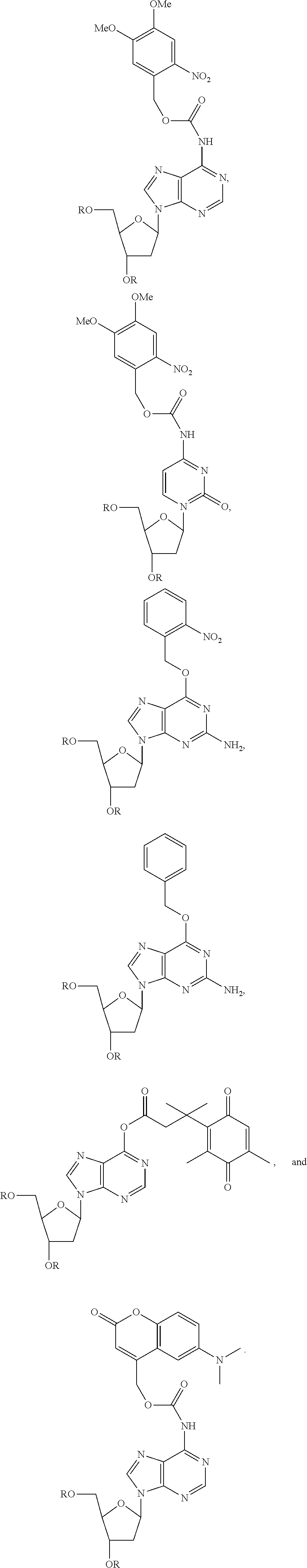

In certain embodiments, the convertible nucleobase is selected from the group consisting of O6-guanine, N2-guanine, N7-guanine, N6-adenine, N5-adenine, O4-thymine, N3-thymine, 2-thio-thymine, 4-thio-thymine, N4-cytosine, or N3-cytosine.

In certain embodiments, the first state and the second state of the plurality of convertible nucleobases are readable by a sequencing method capable of detecting and differentiating non-naturally occurring and/or modified nucleobases.

In certain embodiments, the first state and the second state of the plurality of convertible nucleobases are readable by nanopore sequencing.

In certain embodiments, the first state and the second state of the plurality of convertible nucleobases are readable by sequencing by synthesis.

In certain embodiments, when the plurality of convertible nucleobases are converted to the second state, properties of the plurality of convertible nucleobases are modified (e.g., having reduced size, altered shape, modified H-bonding, and/or modified polymerase substrate ability) as compared to the first state.

In certain embodiments, one or more of the plurality of convertible nucleobases are capable of being converted from the second state into a third state; wherein the one or more of the plurality of convertible nucleobases are attached covalently to the nucleic acid polymer in the third state.

In certain embodiments, each of the plurality of convertible residues is capable of being independently and selectively converted.

In certain embodiments, the polymers provided herein further comprise a plurality of spacer residues linked via the backbone of the polymer, wherein each of the plurality of convertible residues are separated by one or more spacer residues of the plurality of spacer residues.

In certain embodiments, the iterative spacing among the plurality of convertible residues conforms to a resolution of a writing mechanism for encoding data on the polymer.

In certain embodiments, the iterative spacing among two adjacent convertible residues is equal to or greater than a resolution of a data encoding mechanism for encoding data into the polymer.

In certain embodiments, the resolution of the writing mechanism is at least 1 nm.

In certain embodiments, the plurality of spacer residues do not interfere with reading of the convertible residues.

In certain embodiments, the plurality of spacer residues in the polymer are the same spacer residues.

In certain embodiments, the plurality of spacer residues comprise two or more different spacer residues (e.g., different nucleobases such as different naturally occurring nucleobases).

In certain embodiments, the polymer consists essentially of spacer residues.

In certain embodiments, each of the plurality of convertible nucleobases are separated by 2, 3, 4, 5, 6, 7, 8, 9, 10, 20, 30, 40, or 50 spacer residues.

In certain embodiments, each of the plurality of convertible nucleobases are separated by 6 spacer residues.

In certain embodiments, the plurality of spacer residues are naturally occurring nucleobases, non-naturally nucleobases, tetrahydrofuran abasic residues, or ethylene glycol residues.

In certain embodiments, the plurality of spacer residues are naturally occurring nucleobases.

In certain embodiments, the polymers provided herein further comprise one or more delimiters linked to the backbone of the polymer.

In certain embodiments, each of the one or more delimiters comprises one or more naturally occurring nucleobases or non-naturally nucleobases.

In certain embodiments, the one or more delimiters comprise naturally occurring nucleobases.

In certain embodiments, the one or more delimiters separate two or more adjacent data fields within the polymer.

In certain embodiments, the polymers provided herein further comprise one or more data tags.

In certain embodiments, the one or more data tags comprise one or more naturally occurring nucleobases or non-naturally nucleobases.

In certain embodiments, the polymer is a nucleic acid polymer and the one or more data tags are present at the 5′ or 3′ end of the nucleic acid polymer.

In certain embodiments, the one or more data tags are incorporated to the nucleic acid polymer during the nucleic acid polymer is synthesized, during the plurality of convertible nucleobases are converted to the second state, or via ligation after the plurality of convertible nucleobases are converted to the second state.

In certain embodiments, the polymer can be stored under standard nucleic acid storage protocols.

In certain embodiments, the polymer is a nucleic acid polymer that can be stored in appropriate nuclease-free solution at room temperature, or at a lower temperature (e.g., −20° C.).

In certain embodiments, the polymer can be stored at room temperature without stabilizer.

In another aspect, also provided herein are systems for data writing, comprising:
a writable polymer comprising a plurality of convertible residues iteratively spaced along and covalently linked to the backbone of the polymer, wherein each of the plurality of convertible residues has a first state and is capable of being converted from the first state into a second state, the first state and the second state being different and the plurality of convertible residues in the first state and the second state are readable by a polymerase enzyme; wherein the plurality of convertible residues are attached covalently linked to the polymer in the first state and in the second state; and
a data writing device for writing data on the writable polymer.

In certain embodiments, the writable polymer is a writable nucleic acid polymer and the plurality of convertible residues are convertible nucleobases.

In certain embodiments, the data writing device comprises a nanopore.

In certain embodiments, the data writing device comprises a microscope with a light source.

In certain embodiments, the data writing device converts the plurality of convertible nucleobases into the second state by light pulses, voltage pulses, an enzymatic agent, or a redox agent.

In certain embodiments, the data writing device converts the converts the plurality of convertible nucleobases into the second state by light pulses.

In certain embodiments, the data writing device comprises a light irradiation device.

In another aspect, also provided herein are methods for generating a writable nucleic acid polymer, comprising:
providing a circular single-stranded oligonucleotide template, wherein the circular single-stranded oligonucleotide template is complementary to a repeating data field that comprises convertible nucleobases; and
incubating the circular single-stranded oligonucleotide template in the presence of a nucleic acid primer, a polymerase, and triphosphate nucleotides, wherein the triphosphate nucleotides comprise convertible nucleobases in a first state and are capable of being converted from the first state into a second state, the first state and the second state being different.

In certain embodiments, the circular single-stranded oligonucleotide template comprises nucleobases complementary to the convertible nucleobases, and wherein the complementary nucleobases are iteratively spaced such that the incubation of the template with the nucleic acid primer, the polymerase, and the triphosphate nucleotides provides a nucleic acid polymer comprising a plurality of the convertible nucleobases iteratively spaced along and covalently linked via the backbone of the nucleic acid polymer; wherein the plurality of the convertible nucleobases are covalently linked to the nucleic acid polymer in the first state and in the second state.

In certain embodiments, the repeating data field further comprises spacer nucleobases, and wherein the triphosphate nucleotides further comprise triphosphate spacer nucleotides.

In yet another aspect, provided herein are methods for generating a writable nucleic acid polymer, comprising:
chemically synthesizing a plurality of oligomers, each oligomer comprises a plurality of convertible nucleobases iteratively spaced along and linked via the nucleic acid polymer backbone, wherein each of the plurality of convertible nucleobases has a first state and is capable of being converted from the first state into a second state; wherein the plurality of convertible nucleobases are attached covalently to the nucleic acid polymer in the first state and in the second state, the first state and the second state being different; and
ligating the plurality of oligomers to form the writable nucleic acid polymer

In certain embodiments, each of the plurality of oligomers comprises a plurality of spacer residues linked via the backbone of the nucleic acid polymer, wherein each of the plurality of the convertible nucleobases is separated by one or more spacer residues of the plurality of spacer residues.

In certain embodiments, the ligating step is via chemical ligation.

In certain embodiments, the ligating step is via enzymatic ligation.

In certain embodiments, a complementary DNA splint is used in the ligating step.

In certain embodiments, the method further comprises: annealing a plurality of complements to the oligomers prior to the ligating step.

In yet another aspect, provided herein are methods for writing data onto a writable polymer, comprising:
providing a writable polymer that comprises a plurality of convertible residues iteratively spaced along and covalently linked via the backbone of the polymer, wherein each convertible residues of the plurality of convertible residues has a first state and is capable of being converted from the first state into a second state, the first state and the second state being different and the plurality of convertible residues in the first state and the second state are readable by a polymerase enzyme; and
selectively converting, utilizing a data writing device, one or more of the plurality of convertible residues into the second state such that a data encoded polymer is generated.

In certain embodiments, the writable polymer is a writable nucleic acid polymer and the plurality of convertible residues are convertible nucleobases.

In certain embodiments, the data writing device comprises a nanopore, and the method further comprising: passing the writable polymer through the nanopore of the writing device, wherein the nanopore comprises converts one or more of the plurality of convertible residues into the second state.

In certain embodiments, the nanopore is a plasmonic nanopore that provides light pulses or redox energy to selectively convert convertible nucleobases from the first state into the second state.

In certain embodiments, the data writing device comprises a plasmonic well or channel, and the method further comprising: transferring the writable polymer into the plasmonic well or channel of the data encoding device, wherein the plasmonic well or channel provides light pulses or redox energy to selectively convert convertible nucleobases from the first state into the second state.

In certain embodiments, the data writing device selectively coverts the convertible residues into the second state by light pulses, voltage pulses, an enzymatic agent, or a redox agent.

In certain embodiments, the data writing device selectively converts the converts the convertible residues into the second state by light pulses.

In certain embodiments, the convertible residues become naturally occurring nucleobases after conversion into the second state.

In certain embodiments, the plurality of convertible residues comprise two or more types of convertible residues, wherein a first type of convertible residues are activatable by light of a first wavelength and a second type of convertible residues are activatable by light of a second wavelength.

In certain embodiments, the iterative spacing among the plurality of the convertible residues conforms to a resolution of the data writing device for selectively converting the convertible residues.

In certain embodiments, the selectively converting step does not require specific positioning of the writable polymer.

In certain embodiments, the conversion of the convertible residues into the second state is non-uniform on the data encoded polymer.

In certain embodiments, the conversion of the convertible residues into the second state is not limited to certain positions on the data encoded polymer.

In certain embodiments, the method further comprises stretching or combing the writable polymer (e.g., a writable DNA) on a solid support.

In certain embodiments, the method further comprises visualizing locations of the convertible residues using a dye.

In certain embodiments, the method further comprises locally illuminating or locally exciting the writable polymer.

In certain embodiments, the locally illuminating or locally exciting uses Stimulated Emission Depletion (STED) laser.

In certain embodiments, the method further comprises joining two or more data fields from two or more writable polymers end-to-end, resulting in a joined polymer comprising two or more data fields.

In certain embodiments, the method further comprises controlling the passage rate of the writable polymer through the nanopore of the writing device.

In certain embodiments, a plurality of writable polymers pass through the data writing device to write the same data (e.g., generating data redundancy).

In yet another aspect, also provided herein are methods for reading data from a polymer encoded with data, comprising:
providing the polymer encoded with data comprising convertible residues iteratively spaced along and covalently linked via the backbone of the polymer, wherein a first subset of the convertible residues are in a first state and a second subset of the convertible residues are in a second state, the first state and the second state being different and the plurality of convertible residues in the first state and the second state are readable by a polymerase enzyme; and
passing the writable polymer encoded with data through a data reading device to read the encoded data on the polymer encoded with data.

In certain embodiments, the writable polymer is a writable nucleic acid polymer and the plurality of convertible residues are convertible nucleobases.

In certain embodiments, the convertible residues in the first state can be converted into the second state via light.

In certain embodiments, wherein the data reading device comprises a nanopore.

In certain embodiments, wherein the data reading device is a sequencing device.

In certain embodiments, the sequencing device is a sequencing by synthesis device.

In certain embodiments, the method further comprises measuring current flow of electrolytes during passage of the writable polymer.

In certain embodiments, the method further comprises determining whether each of the plurality of convertible residues is in the first state or the second state based on the measured current flow of electrolytes during passage of the writable polymer.

In certain embodiments, the method further comprises re-passing the polymer encoded with data through the data reading device to re-read the encoded data on the polymer encoded with data.

In certain embodiments, the method further comprises validating and correcting the encoded data on the polymer encoded with data by comparing the encoded data on multiple copies of the polymer encoded with data.

In yet another aspect, also provided herein are methods for reading or decoding data from a nucleic acid polymer encoded with data, the method comprising:
providing a plurality of redundant copies of the nucleic acid polymer encoded with data comprising:
   a plurality of converted nucleobases, wherein each converted nucleobase comprises a first nucleobase structure, wherein the first converted nucleobase has been converted from a first state into a second state, the first state and the second state being different; and
   a plurality of convertible nucleobases, wherein each convertible nucleobase comprising a second nucleobase structure and a directly linked leaving group, and wherein the convertible nucleobase is provided in a first state and is capable of being converted from the first state into a second state by releasing the second leaving group from the second nucleobase structure, the first state and the second state being different;
   wherein the converted nucleobases and convertible nucleobases are linked via the nucleic acid polymer backbone; and
sequencing each redundant copy of the plurality redundant copies of the nucleic acid polymer.

In certain embodiments, the method further comprises: detecting the plurality of converted nucleobases and the plurality of convertible nucleobases; and decoding the data based on the detected plurality of converted nucleobases.

In certain embodiments, the plurality of converted nucleobases in the first state and the second state are readable by a polymerase enzyme.

In certain embodiments, the plurality of convertible nucleobases in the first state and the second state are readable by a polymerase enzyme.

In certain embodiments, the plurality of converted nucleobases and the plurality of convertible nucleobases are detected based on the sequencing result of the redundant copies of the nucleic acid polymer encoded with data.

### BRIEF DESCRIPTION OF THE DRAWINGS

The description and claims will be more fully understood with reference to the following figures and data graphs, which are presented as exemplary embodiments and should not be construed as a complete recitation of the scope of the disclosure.

FIGS. 1A and 1B provide a schematic of a writable nucleic acid polymer in accordance with various embodiments.

FIGS. 2A and 2B provide a schematic of a data encodable nucleic acid polymer in accordance with various embodiments.

FIGS. 3A-3G show structures of various example convertible nucleobases for use in a writable nucleic acid polymer.

FIG. 4 provides an example of convertible nucleobase O6-nitrobenzyl-guanine in accordance with various embodiments.

FIGS. 5A and 5B show structures of various example nucleotides comprising a convertible nucleobase for use in a writable polymer in accordance with various embodiments.

FIG. 6 provides molecular structure diagrams of various removable groups (e.g., leaving groups) in a convertible nucleobase for use in a writable polymer in accordance with various embodiments.

FIG. 7 provides a schematic of generating a writable nucleic acid polymer utilizing polymerase extension via a rolling circle reaction in accordance with various embodiments.

FIG. 8 provides a schematic of generating a writable nucleic acid polymer utilizing chemical synthesis and ligation in accordance with various embodiments.

FIGS. 9A-9C provide a schematic for encoding data in a writable nucleic acid polymer utilizing a nanopore and light energy in accordance with various embodiments.

FIGS. 10A-10C provide a schematic for encoding data in a data encodable nucleic acid polymer comprising pairs of convertible nucleobases utilizing a nanopore and light energy in accordance with various embodiments.

FIGS. 11A-11C illustrate encoding data in a writable nucleic acid polymer comprising convertible nucleobases utilizing a nanopore and light energy in accordance with various embodiments. FIG. 11A: a writable nucleic acid polymer comprising convertible nucleobases Cₐ and C_{b}; FIG. 11B: the writable nucleic acid polymer passing through a nanopore, certain convertible nucleobases (e.g., a Cₐ on the 3′ end) has been converted by light energy to converted nucleobases (e.g., Cₐ′) as the written state; and FIG. 11C: certain convertible nucleobases Cₐ and C_{b} have been selectively converted to converted nucleobases Cₐ′ and C_{b}′, respectively, resulting in a nucleic acid polymer encoded with data comprising stochastically or irregularly spaced converted nucleobases Cₐ′ and C_{b}′.

FIGS. 12A-12C provide a schematic for encoding data in a writable nucleic acid polymer comprising duads utilizing a nanopore and light energy in accordance with various embodiments.

FIGS. 13A-13C provide molecular structure diagrams of dual-bit convertible nucleobases for use in a writable nucleic acid polymer in accordance with various embodiments.

FIGS. 14A and 14B provide data decoding strategies using a nanopore current-based sequencing (FIG. 14A) and sequencing by synthesis (FIG. 14B) in accordance with various embodiments.

FIG. 15 illustrates an example of encoding a data encodable nucleic acid polymer comprising convertible nucleobases with binary data 1010010, by selectively converting certain T* to T and G* to G, respectively. Certain convertible nucleobases in the data encodable nucleic acid polymer are skipped during the data encoding process, and the resulting nucleic acid polymer encoded with data comprises stochastically and/or irregularly spaced converted nucleobases (e.g., T and G).

### DETAILED DESCRIPTION

Provided herein are compositions of data-encodable polymers (e.g., nucleic acid polymers), and methods and systems thereof, for data encoding/decoding (writing/reading) and data storage. Also provided herein are method of making the polymers (e.g., nucleic acid polymers) described herein.

Turning now to the drawings and data, compositions and systems of nucleic acid data storage, methods of use and methods of synthesis, in accordance with various embodiments, are disclosed. In several embodiments, a system of data storage comprises writable (i.e., data-encodable) nucleic acid polymers having one or more nucleobases that are convertible. Accordingly, a writable nucleic acid polymer is akin to a blank tape that is encodable, wherein the writable nucleic acid polymer is encoded by converting one or more its nucleobases. Nucleobase conversion can be thought of as a binary code, where each convertible nucleobase is akin to a “bit,” unconverted nucleobases are akin to a “0),” and nucleobases that have been converted are akin to a “1.” It should be understood, however, that a binary code is not the only possibility, and codes can be written in ternary, quaternary, or other numeral system code, which can be done utilizing multiple types of convertible bases or performing multiple writings to further alter the state a convertible base. In some embodiments, the conversion of a convertible nucleobase is stable, or permanent, which allows for long-term archiving. In some embodiments, the combination of two convertible nucleotides comprises a “bit”.

In some embodiments, a convertible residue (e.g., a convertible nucleobase) is referred to as a writable “bit,” and a converted residue (e.g., a converted nucleobase such as a native nucleobase) is referred to as a written “bit.”

In some embodiment, the terms “writable” and “data-encodable” are used herein interchangeably. In some embodiment, the terms “writing” and “data encoding” are used herein interchangeably.

In some embodiments, the terms “leaving group” and “removable group” are used herein interchangeably. In some embodiment, when referring to convertible nucleobases, the terms “pair” and “duad” are used herein interchangeably. “Duad,” used herein refers to a pair of different convertible nucleobases (e.g., writable bits) that are located close enough relative to one another in the polymers described herein (e.g., nucleic acid polymers) such that both are exposed to a single writing action or event (e.g. the same pulse of light or the same voltage pulse). Thus, the convertible nucleotides that comprise the duad are closer than the resolution of the writing action or event.

In other embodiments of the systems provided herein, the systems comprise two or more sets of convertible nucleobases (e.g., nucleobases having different structures, such having different chemically modifiable moieties), where nucleobase conversion (e.g., cage group removal off of nucleobase) can be thought of as a binary code, and each convertible nucleobase (or sets of two or more convertible bases) is akin to a writable “bit” of data, and each converted nucleobase (or sets of two more converted nucleobases) is akin to a written “bit” of data. In some embodiments, convertible nucleobases are utilized to encode a data bit, where conversion of a first nucleobase structure (i.e., a first set of convertible nucleobases) is akin to a “0,” and conversion of a second nucleobase structure (i.e., a second set of convertible nucleobases) of the pair is akin to a “1”, and data can be encoded by selective conversion of nucleobases along the polymer (e.g., the nucleic acid polymer). In some embodiments, a pair of convertible nucleobases are utilized to encode data in a writable bit, where conversion of one nucleobase of the pair is akin to a “0.” and conversion of both nucleobases of the pair is akin to a “1” and data can be encoded by nucleobase pair conversions along the polymer. It should be understood, however, that a binary code is not the only possibility, and codes can be written in ternary, quaternary, or other numeral system code, which can be done utilizing multiple types of convertible bases or performing multiple writings to further alter the state a convertible base. In some embodiments, the conversion of a convertible nucleobase is stable for long periods, or permanent, which allows for long-term archiving.

In some embodiments, the nucleic acid polymer is a single-stranded nucleic acid polymer or a double-stranded nucleic acid polymer. In some embodiments, the nucleic acid polymer is a single-stranded nucleic acid polymer. In some embodiments, the nucleic acid polymer is a double-stranded nucleic acid polymer.

Some embodiments are directed towards compositions of writable nucleic acid polymers. Any appropriate nucleic acid polymer can be utilized, including (but not limited to) DNA, RNA, phosphorothioate DNA, glycerol nucleic acids (GNA), threose nucleic acids (TNA). Further, a nucleic acid polymer may be single stranded or double stranded. In several embodiments, a writable nucleic acid polymer comprises a plurality of convertible nucleobases that are linked by a polymer backbone. In certain embodiments, convertible nucleobases are spaced apart to provide spatial resolution such that each nucleobase can be independently and selectively converted in accordance with encoding. In some embodiments, spacer residues linked via the polymer backbone are utilized to provide spaces between the convertible nucleobases. In some embodiments, spacer residues are unreactive to the writing mechanism. In various embodiments, a writable nucleic acid polymer can further include delimiters and/or data tags for labeling the data, each of which can be provided by a particular sequence of nucleobases.

In some embodiments, any appropriate nucleic acid polymer can be utilized, including (but not limited to) DNA, RNA, phosphorothioate DNA, glycerol nucleic acids (GNA), threose nucleic acids (TNA), locked nucleic acids (LNA), and combinations thereof.

In some embodiments, the plurality of convertible nucleotides are capable of being incorporated into the nucleic acid polymer by one or more polymerase enzymes.

In some embodiments, the plurality of convertible nucleobases are non-naturally occurring nucleobases. In some embodiments, the plurality of convertible nucleobases are modified naturally occurring nucleobases or derivatives of naturally occurring nucleobases.

In some embodiments, each of the plurality of convertible nucleobases comprises a chemically modifiable moiety. In some embodiments, each of the plurality of convertible nucleobases the chemically modifiable moiety is directly attached to the base of the convertible nucleobases. In some embodiments, each of the plurality of convertible nucleobases the chemically modifiable moiety is attached to the base without a linker or a sidechain. In some embodiments, the plurality of convertible nucleobases are covalently linked to the backbone of the nucleic acid via a sugar of the backbone of the nucleic acid. In some embodiments, the removable group in the plurality of convertible nucleobases are covalently linked to the backbone of the nucleic acid via the nucleobase.

In some embodiments, the convertible nucleobases are linked to the backbone of the nucleic acid polymer in the same way that a nucleobase in a native nucleotide is linked to the backbone of the nucleic acid polymer (via the sugar in a nucleotide), without an intervening linker or as a sidechain.

In some embodiments, the nucleobase conversion (i.e., from the first state to the second state) is performed by removing one or more removal groups from the nucleobase. In several embodiments, the removable group is a caging group.

In one embodiment, the chemically modifiable moiety is activatable by light, thereby converting from the first state into the second state. In some embodiments, the conversion from the first state into the second state occurs via an irreversible reaction. In some embodiments, the convertible nucleobase becomes a naturally occurring nucleobase after conversion into the second state. In some embodiments, the convertible nucleobase becomes a native nucleobase after conversion into the second state. In one embodiment, the convertible nucleobase becomes guanine, adenine, thymine, uracil, or cytosine after conversion into the second state. In some embodiments, the backbone of the polymer (e.g., phosphate and sugar in nucleic acid polymer) remain unchanged during the conversion from the first state into the second state. In some embodiments, the chemically modifiable moiety is activatable by light, voltage, enzymatic agent, chemical reagent, or a redox agent or redox electrode, thereby converting from the first state into the second state. In some embodiments, the chemically modifiable moiety comprises one or more photo-removable groups.

In some embodiments, the one or more photo-removable groups are:

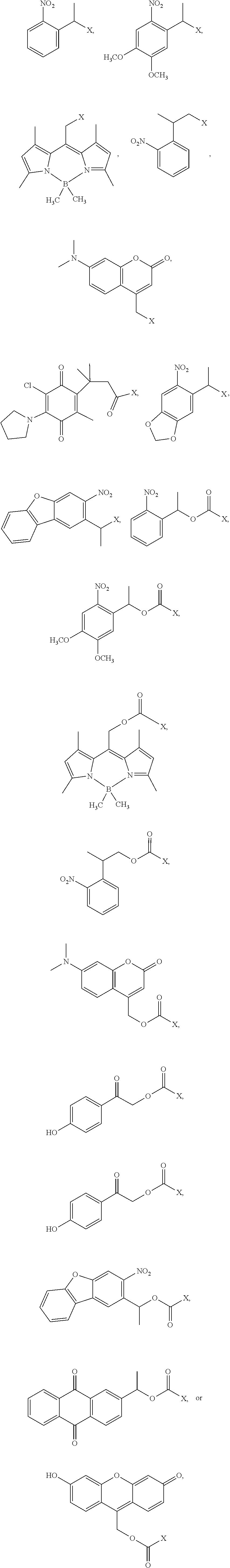

wherein X represents NR2, NHR, OR, or SR, and wherein R is the nucleobase to which the photo-removable group is attached.

In some embodiments, the plurality of convertible nucleobases are capable of being converted by light of a wavelength of 325 nm, 360 nm, or 400 nm.

In some embodiments, the plurality of convertible nucleobases are capable of being converted by light of a wavelength of between 400 nm to 850 nm.

In some embodiments, each of the plurality of convertible nucleobases comprises a chemically modifiable moiety that is activatable or removable by redox. In some embodiments, the chemically modifiable moiety is capable of being activated by localized oxidation. In some embodiments, the chemically modifiable moiety is capable of being activated by oxidation or reduction using one or more electrodes.

In some embodiments, a nucleotide comprising the convertible nucleobase is selected from the group consisting of:

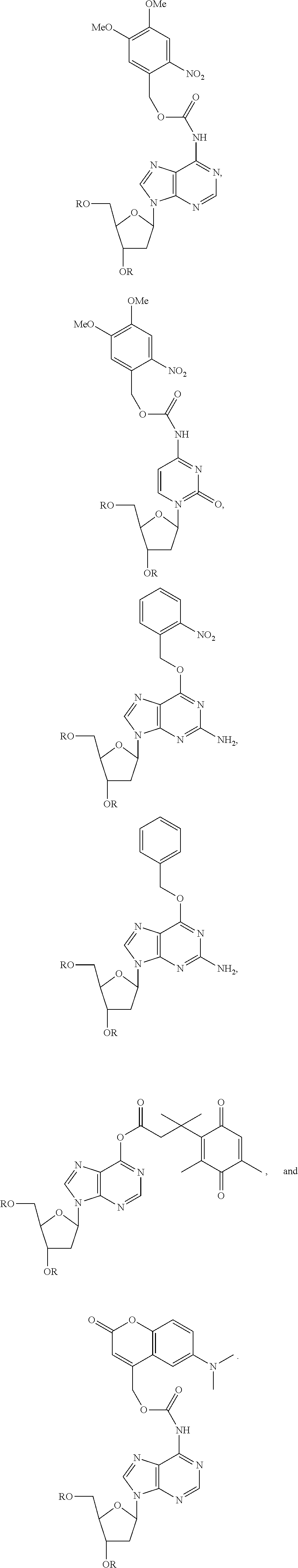

In some embodiments, the convertible nucleobase (with a specific substitution position of the removable group) is selected from the group consisting of O6-guanine, O6-thioguanine, N2-guanine, N7-guanine, N6-adenine, N5-adenine, O4-thymine, O4-uracil, N3-thymine, 2-thio-thymine, 4-thio-thymine, N4-cytosine, or N3-cytosine.

In some embodiments, the first state and the second state of the plurality of convertible nucleobases are readable by a sequencing method capable of detecting and differentiating non-naturally occurring and/or modified nucleobases. In some embodiments, the first state and the second state of the plurality of convertible nucleobases are readable by nanopore sequencing. In some embodiments, the first state and the second state of the plurality of convertible nucleobases are readable by sequencing by synthesis. In some embodiments, when the plurality of convertible nucleobases are converted to the second state, properties of the plurality of convertible nucleobases are modified (e.g., having reduced size, altered shape, modified H-bonding, and/or modified polymerase substrate ability and/or polymerase coding) as compared to the first state. In some embodiments, one or more of the plurality of convertible nucleobases are capable of being converted from the second state into a third state; wherein the one or more of the plurality of convertible nucleobases are attached covalently to the nucleic acid polymer in the third state. In some embodiments, each of the plurality of convertible residues is capable of being independently and selectively converted.

In some embodiments, the polymers described herein (e.g., nucleic acid polymers) comprise two or more different sets of convertible residues, each set of convertible residues has a first state and is capable of being converted from the first state into a second state, the first state and the second state being different. In some embodiments, each of the plurality of convertible residues comprises a chemically modifiable moiety that can be activated and/or removed by light, and the two or more different sets of convertible residues are activatable and/or removable by light of different wavelengths. In some embodiments, a first set of convertible residues is activatable by light of a first wavelength, and a second set of convertible residues is activatable by light of a second wavelength, the first wavelength and the second wavelength being different.

In certain embodiments, the convertible nucleobases (or pairs of convertible bases) in the writable nucleic acid polymers described herein are iteratively spaced apart to provide spatial resolution such that each nucleobase (or each set or pair) can be independently and selectively converted in accordance with encoding. In certain embodiments, the convertible nucleobases are regularly or irregularly spaced apart, but data is encoded by identifying and selectively converting certain nucleobases to yield a nucleic acid polymer encoded with data. In some of the embodiments, the data encoding mechanism may skip any convertible nucleobases as necessary until it reaches the right convertible nucleobase in accordance with the code.

In some preferred embodiments, the convertible nucleobases are regularly spaced apart (e.g., by spacers), but data is encoded by identifying and selectively converting certain nucleobases to yield a nucleic acid polymer encoded with data comprising stochastically spaced converted nucleobases (i.e., written bits). One of the advantages of the writable nucleic acid polymers provided herein is no controlling of the position or passing rate of the writable nucleic acid polymers is needed. Certain convertible nucleobases can be skipped.

In several embodiments, a writing procedure is utilized to encode a writable nucleic acid with data. Data encoding can be performed by selectively converting convertible nucleobases of a nucleic acid molecule such that the written nucleic acid molecule contains a sequence of unconverted and converted nucleobases, akin to a binary code of “zeros” and “ones”. Any appropriate mechanism to chemically convert a nucleobase into second structure can be utilized. In accordance with various embodiments, a nucleobase is altered via light, voltage, enzymatic agent, chemical reagent, and/or a redox agent.

In some embodiments, the data written (data-encoded) nucleic acid molecule contains a sequence of converted nucleobases comprising a converted first set of nucleobases and a converted second set of nucleobases, akin to a binary code of “zeros” and “ones”.

In some embodiments, the data written (encoded) nucleic acid polymers are stored in accordance with standard nucleic acid storage protocols. For instance, data written nucleic acid polymers can be stored dry, as a precipitate, or in an appropriate nuclease-free solution at room temperature, or at colder temperatures (e.g., 20° C.). Stabilizers such as (for example) alcohol, chelating agents and nuclease inhibitors, may be included with the stored nucleic acid. To read the data on written nucleic acid polymers, any appropriate sequencer capable of reading unnatural and/or altered nucleobases can be utilized, such as Oxford Nanopore Technologies PromethION, MinION, and GridION sequencing platforms (Oxford. UK) or Pacific Bioscience's Single Molecule. Real-Time (SMRT) sequencing platform (Menlo Park. CA). Alternatively, a nanopore device can be fabricated or manufactured for reading the data. The nanopore can be comprised of solid-state materials, or can contain one or more proteins.

In some embodiments, the use of solid supports to sequester and stabilize the nucleic acid such as polymer beads, glass beads, or mineral solids are also contemplated. In some embodiments, the data on the written (encoded) nucleic acid polymers is decoded or read by sequencing by synthesis (SBS). And in some embodiments, a sequencer capable of reading modified and/or unmodified nucleobases can be utilized to decode or read data, such as Oxford Nanopore Technologies PromethION. MinION, and GridION sequencing platforms (Oxford. UK) or Pacific Bioscience's Single Molecule. Real-Time (SMRT) sequencing platform (Menlo Park. CA).

The present disclosure overcomes many of the limitations associated with traditional nucleic acid data storage by separating the synthesis and data encoding into distinct steps. The disclosure provides molecular strategies for producing long strands of writable nucleic acids that, in themselves, do not encode data, but rather provide a template with the capacity for being written. Writable nucleic acid polymers can be produced in bulk in advance of data encoding. The disclosure further provides compositions and systems comprising convertible nucleobases (and pairs of convertible nucleobases) that act as writable “bits” of data, which can be switched from a first state into a second state, thus defining “0” and “1” in binary code. The disclosure further provides methods for writing data into the writable nucleic acid polymers provided herein at the single molecule level, thus consuming negligible amounts of material. Data writing may be achieved chemically or physically, utilizing (for example) light pulses or voltage pulses. Finally, because the written nucleic acid polymers are long, they encode more data per molecule than do short DNAs, and can be efficiently and rapidly read by various sequencers existing within the current market. The compositions, systems, and methods described herein greatly increase the speed and density of nucleic acid data encoding while lowering cost.

### Writable Polymers for Encoding Data

In one aspect, provided herein are polymers for encoding data, comprising a plurality of convertible residues, iteratively spaced along and covalently linked to the backbone of the polymer, wherein each of the plurality of convertible residues has a first state and a is capable of being converted from the first state into a second state, and wherein the plurality of convertible residues are covalently linked to the polymer in the first state and in the second state. In some embodiments, the first state and the second state are different (e.g., the convertible residues have different structures when in the first and the second state). In some embodiments, the plurality of convertible residues in the first state and in the second state are readable by a polymerase enzyme. In some embodiments, the plurality of convertible residues are repeatedly spaced along the backbone of the polymer.

In some embodiments, the polymers described herein are nucleic acid polymers and the plurality of convertible residues are convertible nucleobases.

In certain embodiments, the convertible residues are iteratively spaced apart to provide spatial resolution such that each residue can be independently converted. In some embodiments, any appropriate spacer (e.g., non-writable, i.e., unreactive to the data writing mechanism) are between the convertible residues. In some embodiments, residues linked by the polymer backbone can be utilized as spacers. In some embodiments, the spacers spaced between the convertible residues in accordance with the spatial resolution of the writing mechanism and/or writing device. In some embodiments, spacers are residues, which may be unreactive to the writing mechanism. In some embodiments, these spacers are unmodified DNA nucleotides. In various embodiments, the polymer further comprises delimiters and/or data tags for labeling the data.

In some embodiments, the polymers described herein (e.g., nucleic acid polymers) further comprise a plurality of spacer residues linked via the backbone of the polymer, wherein each of the plurality of convertible residues are separated by one or more spacer residues of the plurality of spacer residues. In some embodiments, wherein the iterative spacing among the plurality of convertible residues conforms to a resolution of a writing mechanism for encoding data on the polymer. In some embodiments, the iterative spacing among two adjacent convertible residues is equal to or greater than a resolution of a data encoding mechanism for encoding data into the polymer. In some embodiments, the resolution of the writing mechanism is at least 1 nm. In some embodiments, the plurality of spacer residues do not interfere with reading of the convertible residues. In some embodiments, the plurality of spacer residues in the polymer are the same spacer residues. In some embodiments, the plurality of spacer residues comprise two or more different spacer residues (e.g., different nucleobases such as different naturally occurring nucleobases).

In some embodiments, the polymers described herein are blank tapes. In some embodiments, the polymers described herein are blank tapes of DNA. Blank tape used herein refers to a writable nucleic acid polymer that comprises convertible nucleobases iteratively spaced along the writable nucleic acid polymer, such that conversion of convertible nucleobases from a first state into a second state results in encoding of data. The blank tape itself contains no data, but is capable of being encoded with data by use of an appropriate writing system (e.g., by light) via converting the convertible nucleobases. In some embodiments, the blank tape is writable sequentially from one end to the other end to encode data.

In some embodiments, the blank tape is writable over its entire length. In some embodiments, each convertible nucleobase in the blank tape is independently and individually writable.

In some embodiments, the polymers described herein (e.g., nucleic acid polymers) consist essentially of spacer residues.

In some embodiments, the polymers described herein (e.g., nucleic acid polymers) comprise no delimiter or data tag.

In some embodiments, the polymers described herein (e.g., nucleic acid polymers) consist of spacer residues and convertible residues (e.g., convertible nucleobases).

In some embodiments, each of the plurality of convertible nucleobases are separated by 2, 3, 4, 5, 6, 7, 8, 9, 10, 20, 30, 40, or 50 spacer residues. In some embodiments, each of the plurality of convertible nucleobases are separated by 6 spacer residues. In some embodiments, the plurality of spacer residues are naturally occurring nucleobases, non-naturally nucleobases, tetrahydrofuran abasic residues, or ethylene glycol residues, the plurality of spacer residues are naturally occurring nucleobases.

In some embodiments, the polymers described herein (e.g., nucleic acid polymers) further comprise one or more delimiters linked to the backbone of the polymer. In some embodiments, each of the one or more delimiters comprises one or more naturally occurring nucleobases or non-naturally nucleobases. In some embodiments, the one or more delimiters comprise naturally occurring nucleobases. In some embodiments, the one or more delimiters separate two or more adjacent data fields within the polymer.

In some embodiments, the polymers described herein (e.g., nucleic acid polymers) further comprise one or more data tags. In some embodiments, the one or more data tags comprise one or more naturally occurring nucleobases or non-naturally nucleobases. In some embodiments, the polymer is a nucleic acid polymer and the one or more data tags are present at the 5′ or 3′ end of the nucleic acid polymer. In some embodiments, the one or more data tags are incorporated to the nucleic acid polymer during the nucleic acid polymer is synthesized, during the plurality of convertible nucleobases are converted to the second state, or via ligation after the plurality of convertible nucleobases are converted to the second state.

In some embodiments, the polymer can have any number or length of monomeric units, for example, from as short as 10 monomeric units to longer than 100,000 monomeric units. In various embodiments, the polymer has greater than 500 monomeric units, greater than 1,000 monomeric units, greater than 5000 monomeric units, greater than 10,000 monomeric units, greater than 50,000 monomeric units, or greater than 100,000 monomeric units.

In some embodiments, the nucleic acid polymer comprises greater than 10 convertible residues. In some embodiments, the nucleic acid polymer comprises greater than 100 convertible residues. In some embodiments, the nucleic acid polymer comprises greater than 500 convertible residues. In some preferred embodiments, the nucleic acid polymer comprises greater than 1,000 convertible residues. In some embodiments, the nucleic acid polymer comprises greater than 10,000 convertible residues. In some embodiments, the nucleic acid polymer comprises greater than 100,000 convertible residues.

In some embodiments, the ratio of the total number of monomeric units (e.g., nucleotides) to the convertible residues (e.g., convertible nucleobases) in the polymer (e.g., nucleic acid polymer) is between 2 to 500. In some embodiments, the ratio of the total number of monomeric units (e.g., nucleotides) to the convertible residues (e.g., convertible nucleobases) in the polymer (e.g., nucleic acid polymer) is between 2 to 200. In some embodiments, the ratio of the total number of monomeric units (e.g., nucleotides) to the convertible residues (e.g., convertible nucleobases) in the polymer (e.g., nucleic acid polymer) is between 2 to 100. In some embodiments, the ratio of the total number of monomeric units (e.g., nucleotides) to the convertible residues (e.g., convertible nucleobases) in the polymer (e.g., nucleic acid polymer) is between 2 to 10. In some embodiments, the ratio of the total number of monomeric units (e.g., nucleotides) to the convertible residues (e.g., convertible nucleobases) in the polymer (e.g., nucleic acid polymer) is between 10 to 50).

In some embodiments, the ratio of the total number of monomeric units (e.g., nucleotides) to the convertible residues (e.g., convertible nucleobases) in the polymer (e.g., nucleic acid polymer) is between 10 to 100. In some embodiments, the ratio of the total number of monomeric units (e.g., nucleotides) to the convertible residues (e.g., convertible nucleobases) in the polymer (e.g., nucleic acid polymer) is between 20 to 100. In some embodiments, the ratio of the total number of monomeric units (e.g., nucleotides) to the convertible residues (e.g., convertible nucleobases) in the polymer (e.g., nucleic acid polymer) is between 20 to 50. In some embodiments, the ratio of the total number of monomeric units (e.g., nucleotides) to the convertible residues (e.g., convertible nucleobases) in the polymer (e.g., nucleic acid polymer) is greater than 100.

### Writable Nucleic Acid Polymers

In certain embodiments, the polymers described herein (e.g., writable polymers) are nucleic acid polymers and the plurality of convertible residues are convertible nucleobases. In certain embodiments, the polymers described herein are nucleic acid polymers comprising a plurality of convertible nucleobases iteratively spaced along and covalently linked to the backbone of the nucleic acid polymer, wherein each of the plurality of convertible nucleobases has a first state (e.g., having a first state structure) and is capable of being converted from the first state into a second state (e.g., having a second state structure), the plurality of convertible nucleobases are covalently linked to the nucleic acid polymer in the first state and in the second state. In some embodiments, the first state and the second state are different and are both readable by a polymerase enzyme. In some embodiments, the nucleobase in the second state is a natural nucleobase. In some embodiments, the nucleobase in the second state is scarless (i.e., in native form of nucleobase, such as guanine, adenine, thymine, thiothymine, thioguanine, or 5-methylcytosine, or cytosine.

In some embodiments, the unwritten state is also referred to as the unconverted state, and the written state is also referred to the converted state.

Compounds in accordance with embodiments of the disclosure are based on nucleic acids having a plurality of convertible nucleobases, which are akin to writable data bits. Each convertible nucleobase can exist in two or more states, an unwritten state (e.g., a first state) akin to a “0”, and at least a first written state (e.g., a second state of the nucleobase) akin to a written bit denoting “1”, and in some embodiments a second written state (e.g., a third state of the nucleobase), and/or further written states (i.e., the written bits are further writable). In several embodiments, the writable nucleic acid polymers are synthesized with a plurality of convertible nucleobases in an “unwritten” state that are capable of being converted to “written” state(s). In some embodiments, two different convertible nucleobases are employed as a pair for encoding a single bit; conversion of one encodes a “0” while conversion of the other encodes a “1”. These writable nucleic acids can be created having long lengths (e.g., 5 to 50 kb, or more) and can be produced in bulk, prior to data writing.

In some embodiments, a single convertible nucleobase is utilized to encode a bit of data. In some embodiments, a set of two or more convertible nucleobases is utilized to enable the encoding of a bit of data. In some embodiments, a pair of two different convertible nucleobases are employed as a pair for enabling the encoding of a single bit. In some embodiments utilizing a pair of two different convertible nucleobases, conversion of a first nucleobase encodes a “0” while conversion of the other nucleobase encodes a “1”. In some embodiments utilizing a pair of two different convertible nucleobases, conversion of one nucleobase encodes a “0” while conversion of both of the nucleobases encodes a “1”.

In several embodiments, the writable nucleic acid polymer comprises a plurality of convertible nucleobases that are linked to the polymer backbone. In certain embodiments, convertible nucleobases are iteratively spaced apart to provide spatial resolution such that each nucleobase can be independently converted. In some embodiments, the spatial resolution depends, at least in part, on the writing mechanism. For instance, if an optical light source and device with 1 nm of resolution is used to alter nucleobases, then each convertible base needs to be separated by at least 1 nm. Any appropriate spacer between the alterable nucleobases can be utilized. In some embodiments, residues linked by the polymer backbone can be utilized as spacers. Because the distances between nucleobases in a double-stranded DNA polymer is about 0.34 nm, in accordance with numerous embodiments, three spacers are utilized for each nanometer of spatial resolution of the alteration-inducing source. In some embodiments, spacers are nucleobases, which may be unreactive to the writing mechanism. In various embodiments, a writable nucleic acid polymer can further include delimiters and/or data tags for labeling the data, each of which can be provided by a particular sequence of residues.

In several embodiments, a data encodable nucleic acid polymer comprises a plurality of convertible nucleobases that are linked by the polymer backbone. In certain embodiments, convertible nucleobases are regularly or irregularly spaced apart, but data is encoded by identifying and selectively converting nucleobases to yield an encoded polymer. In some of the embodiments utilizing regularly or irregularly spaced convertible nucleobases, the data encoding mechanism may skip any convertible nucleobases as necessary until it reaches the right convertible nucleobase in accordance with the code, resulting in a nucleic acid polymer encoded with data comprising stochastically and/or regularly spaced converted nucleobases. In certain embodiments, convertible nucleobases (or sets of nucleobases) are iteratively spaced apart to provide spatial resolution such that each nucleobase (or each set of nucleobases) can be independently converted. The spatial resolution depends, at least in part, on the writing mechanism. For instance, if an optical light source and device with 1 nm of resolution is used to alter nucleobases, then each convertible base (or each set of nucleobases) needs to be separated by at least 1 nm. Any appropriate spacer between the convertible nucleobases (or sets of nucleobases) can be utilized. In some embodiments, residues linked by the polymer backbone can be utilized as spacers. Because the distances between nucleobases in a double-stranded DNA polymer is about 0.34 nm, in accordance with numerous embodiments, three spacers are utilized for each nanometer of spatial resolution of the alteration-inducing source. In some embodiments, spacers are nucleobases, which may be unreactive to the writing mechanism. In various embodiments, a data encodable nucleic acid polymer can further include delimiters and/or data tags for labeling the data, each of which can be provided by a particular sequence of residues.

In some embodiment, the writable nucleic acid polymers provided herein are capable of being written (e.g., convertible nucleobases selectively and sequentially converted to converted (e.g., naturally occurring or native nucleobases)) in both directions (e.g., in either the 5′ to 3′ direction or the 3′ to 5′ direction).

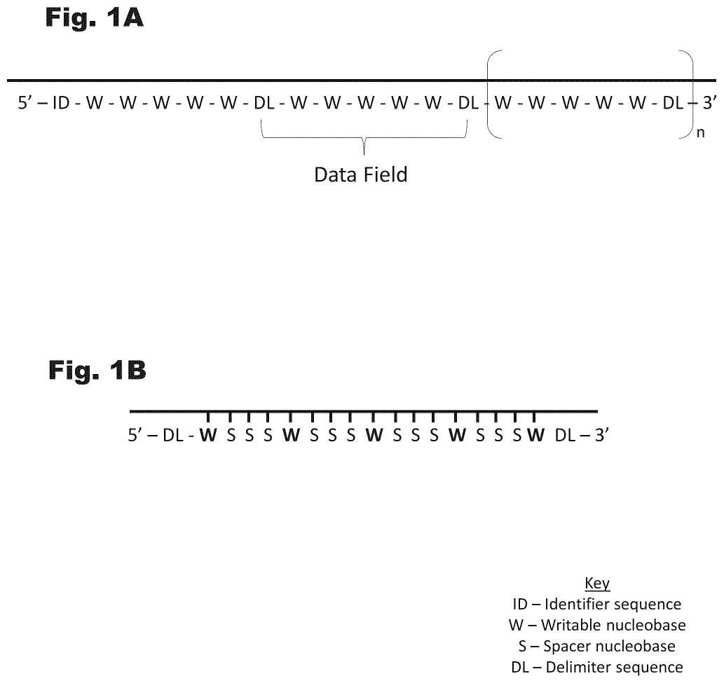

FIG. 1A illustrates an example of a writable nucleic acid polymer having a plurality of writable nucleobases. The writable nucleic acid polymer comprises a repeating strand sequence, which can exist as a single-stranded or double-stranded molecule. The repeating unit comprises convertible nucleobases, which may be natural or unnatural, that can undergo chemical changes from a first structure state to a second structure state, akin to a switch from a “0” state to a “1” state. Each of these convertible bases is akin to a “bit” for data encoding. It is understood that the definition of “1” and “0” is arbitrary, and simply meant to signify binary code. Prior to any data writing, convertible nucleobase bases are initially provided in the unconverted state. In some embodiments, the repeating unit of the writable nucleic acid polymer comprises data fields that include a plurality of convertible nucleobases, and may also contain spacers or sequences that delimit or separate bits. FIG. 1B provides another example of a data field sequence having a plurality of convertible nucleobases separated by spacers. For example, as shown, three spacers are utilized between each convertible nucleobase which would provide 1 nm of spatial resolution. It is understood that longer spacer sequences can be used in cases of lower bit-writing resolution. In some embodiments, a writable nucleic acid polymer includes one or more unique data tag sequences, denoting documentation such as type of data, date, or other information. A unique data tag sequence may be written during the synthesis of the writable DNA, or may be written during the data writing process, or may be added on to an end via a primer, or may be added to the data strand via ligation after data writing.

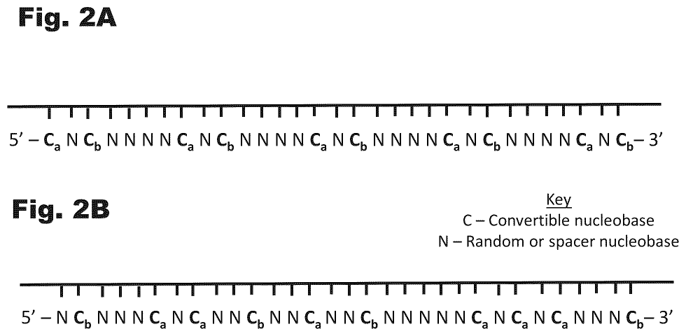

FIG. 2A illustrates yet another example of a data encodable nucleic acid polymer having a plurality of convertible nucleobases in which each bit is a pair of convertible nucleobases that are iteratively repeated along the polymer. The data encodable nucleic acid polymer can exist as a single-stranded or double-stranded molecule. Each convertible nucleobase contains a removable group such that the nucleobase can be converted from one structure state to a second structure state by removing the removable group via light or redox energy. In reference to FIG. 2A, in some embodiments, conversion of the “Cₐ” nucleobase yet maintaining the “C_{b}” unconverted yields a “zero” bit and conversion of the “C_{b}” nucleobase yet maintaining the “Cₐ” unconverted yields “one” bit. In some embodiments, conversion of the “Cₐ” nucleobase yet maintaining the “C_{b}” unconverted yields a “zero” bit and conversion of both the “Cₐ” and “C_{b}” nucleobases yields a “one” bit. It is understood that the definition of “zero” and “one” is arbitrary, and simply meant to signify binary code.

FIG. 2B illustrates a further example of a data encodable nucleic acid polymer having a plurality of convertible nucleobases in which each bit is a convertible nucleobase that are spaced along the nucleic acid polymer. The data encodable nucleic acid polymer can exist as a single-stranded or double-stranded molecule. Each convertible nucleobase contains removal group such that the nucleobase can be converted from one structure state to a second structure state by removing the removable group via light or redox energy. As shown in FIG. 2B, in some embodiments, conversion of the “Cₐ” nucleobase yields a “zero” bit and conversion of the “C_{b}” nucleobase yields “one” bit. In these embodiments, convertible nucleobases can be left unconverted and thus do not contribute to the code of data.

In some embodiments, a data encodable nucleic acid polymer includes one or more unique data tag sequences, denoting documentation such as type of data, date, or other information. A unique data tag sequence may be incorporated during the synthesis of the encodable polymer, or may be added on to an end via a primer, or may be added to the data strand via ligation after data encoding.

In various embodiments, writable nucleic acid polymers can be any length, for example, from as short as 15 nucleotides to longer than 100 kilobases. In various embodiments, a writable nucleic acid polymer is greater than 500 nucleotides long, is greater than 1000 nucleotides, is greater than 5000 nucleotides, is greater than 10,000 nucleotides, is greater than 50,000 nucleotides, or is greater than 100,000 nucleotides. Maximum lengths are only limited by the stability of the DNA, by the method used to make them, and by the method used to read the written data. In some embodiments, longer strands have the advantage of containing more data per molecule. Notably, current sequencing technologies can handle nucleic acid strands of tens to hundreds of thousands of bases in length (see N Kono and K. Arakawa, Dev Growth Differ. 2019:61:316-326; and Q Chen and Z. Liu, Sensors (Basel). 2019:19:1886; the disclosures of which are each incorporated herein by reference).

Several embodiments are directed to convertible nucleobases, which can be incorporated into a writable nucleic acid polymer. A convertible nucleobase, in accordance with various embodiments, is a nucleic acid base that is capable of being converted from a first chemical state into a second chemical state by a controlled reaction chemistry. Any appropriate mechanism to convert a nucleobase from a first state into a second state can be utilized, including (but not limited to) light pulses, voltage pulses, enzymatic agent, chemical reagent, and/or redox agent. It is understood that “nucleobases” are not limited to naturally occurring structures, but may also embody unnatural nucleobases, such as designer nucleobases.

In some embodiments, the convertible nucleobases are nucleic acid bases that are capable of being converted from a first structural state into a second structural state by a controlled reaction chemistry. In some embodiments, a convertible nucleobase comprises a removable group that can be removed (e.g., as a leaving group) to provide a structural change. Any appropriate mechanism to convert a nucleobase from a first state into a second state can be utilized, including (but not limited to) light pulses, voltage pulses, enzymatic agent, chemical reagent, and/or redox agent. It is understood that “nucleobases” are not limited to naturally occurring structures, but may also embody unnatural nucleobases, such as designer nucleobases.

In some embodiments, the structural change results in a conversion of a non-natural nucleobase (e.g., nucleobase in the first structural state) to a natural or native nucleobase (e.g., nucleobase in the second structure state). A natural or native nucleobase in this definition can be identified by standard sequencing methods. In some embodiments, the nucleobase in the second state is a natural nucleobase. In some embodiments, the nucleobase in the second state has no scar. In some embodiments, the nucleobase in the first state comprises a chemically modifiable moiety. In some embodiments, the nucleobase in the first state does not comprise a linker (or a linker moiety) or a sidechain between the base of the nucleobase and the chemically modifiable moiety. In some embodiments, when the nucleobase in the first state is converted to the second state, the chemically modifiable moiety is removed, thereby leaving the nucleobase in the second state a natural or native nucleobase. In some embodiments, the nucleobase in the first state and in the second state are readable or recognizable by polymerase. In some embodiments, the written nucleic acid polymer is readable by various sequencing methods, e.g., sequencing by synthesis (SBS).

In some embodiments, “scar” used herein refers to a group not normally found on naturally occurring DNA (such as a portion of a linker or a sidechain) that remains behind after a covalent bond is cleaved. Scars are frequently observed in some DNA sequencing technologies where a label is released by cleaving a linker during sequencing steps.

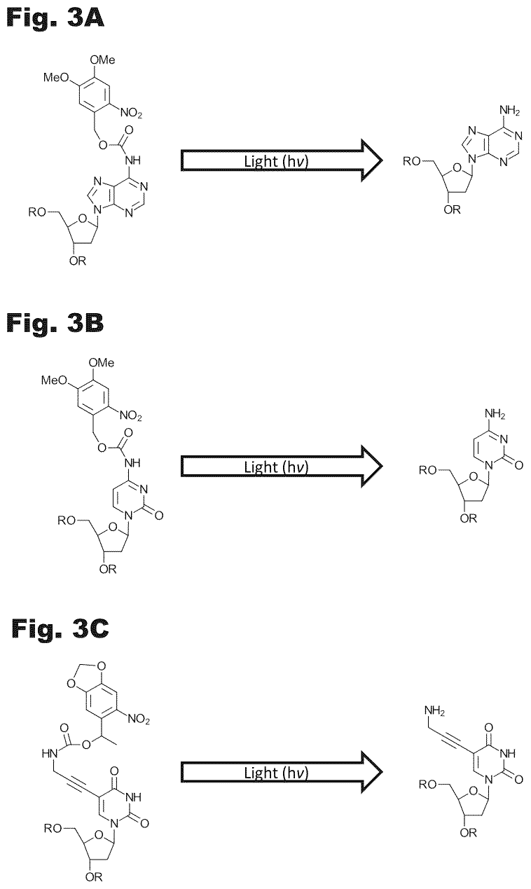

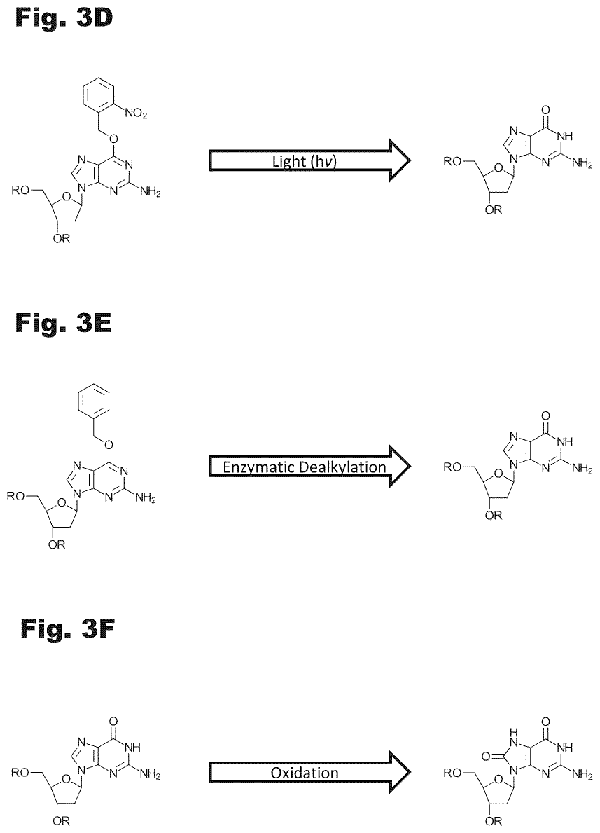

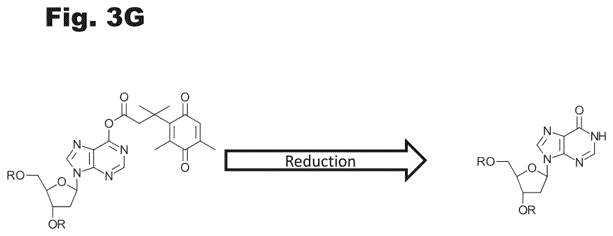

Provided in FIGS. 3A-3G are examples of convertible nucleobases in their unconverted and converted states. In several embodiments, convertible nucleobases can encode “bits” of data, enabling conversion from a first structure state to a second structure state, akin to “0” or “1” digital bit designations. In some embodiments, each state of the nucleobase is to be readable by sequencing methods capable of detecting and differentiating unnatural and/or modified bases, such as (for example) sequencing by synthesis or nanopore sequencing. As provided in FIGS. 3A-3G are examples of convertible nucleobases designed to be converted from a first state into a second state by localized pulses of light, which remove caging groups, reducing the size, altering shape or H-bonding of the base. Various photo removable groups can be incorporated into light convertible nucleobases (see, e.g., D. D. Young and A. Deiters. Org Biomol Chem. 2007:5:999-1005; and Y. Wu. Z. Yang, and Y. Lu. Curr Opin Chem Biol. 2020:57:95-104; the disclosures of which are each incorporated herein by reference). While a few examples are provided, it is understood that any appropriate photo-removable group and other nucleobases may be used in accordance with the various embodiments. FIG. 3E provides a convertible nucleobase that can be converted by localized enzymatic activity that removes a group resulting in altered size, shape, and H-bonding (see A. E. Pegg and T. L. Byers. FASEB J 1992:6:2302-10. FIG. 3F provides a convertible nucleobase that is converted by localized oxidation, resulting in an altered shape and polymerase substrate capability (K. Kino, et al., Genes Environ. 2017; 39:21). FIG. 3G provides a convertible nucleobase that is converted with a redox-removable group, again resulting in an altered size, shape, and/or polymerase substrate ability. In FIGS. 3A-3G, both the unconverted state and converted state of these nucleobase are uniquely identifiable by current sequencing methods.

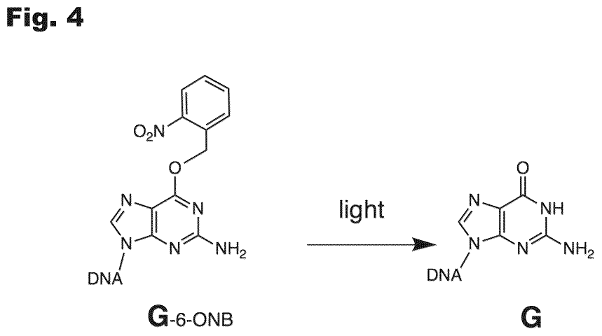

FIG. 4 illustrates the conversion of a convertible nucleobase O6-nitrobenzyl-guanine to guanine by using light energy to break the bond with the nitrobenzyl group. This conversion can represent a bit of data or can be utilized in combination with one or more other convertible nucleobases to represent a writable bit of data. When decoding data via sequencing by synthesis, an unconverted O6-nitrobenzyl-guanine will be read as a mix of A and G and after conversion, the resulting guanine will be read as >99% G.

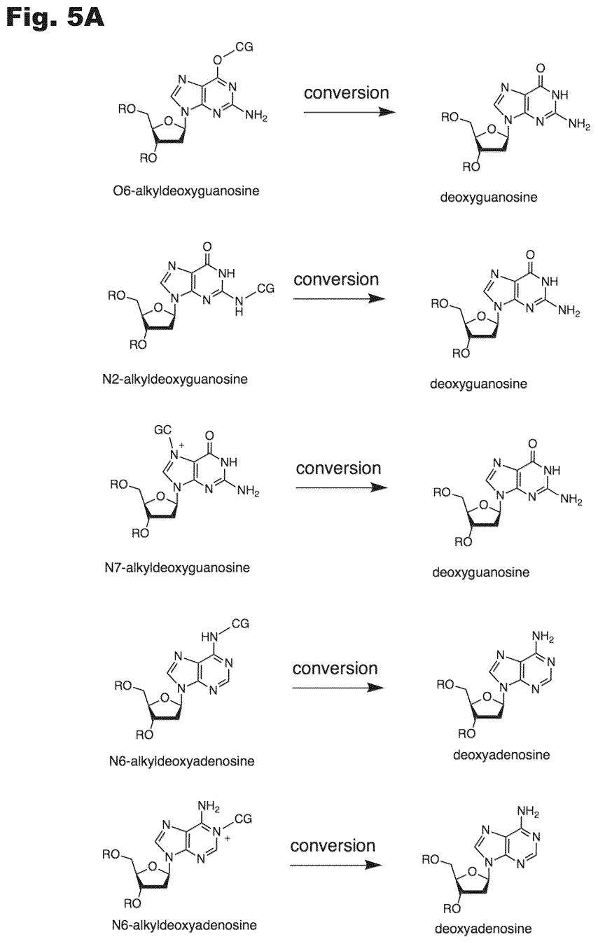

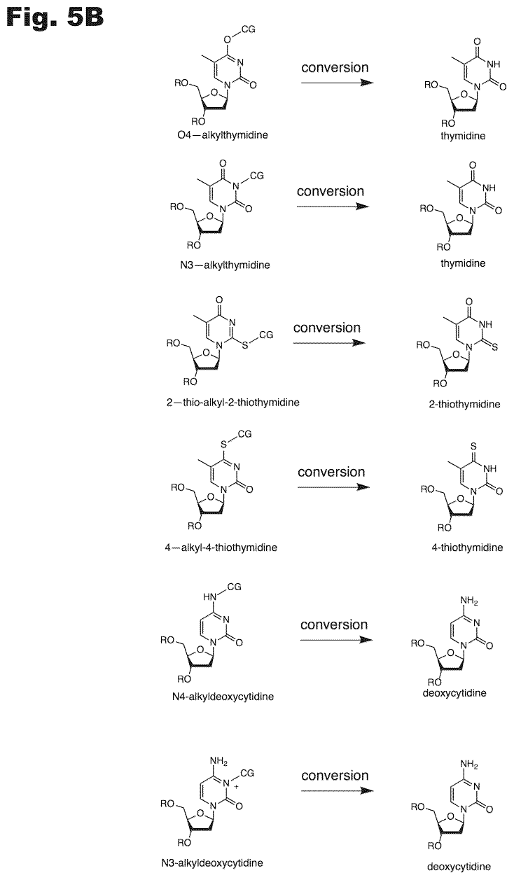

FIGS. 5A-5B show more examples of convertible nucleobases that can be converted from a first state into a second state by localized pulses of light, which remove caging groups, yielding natural nucleobase structures. Each exemplary convertible nucleobase includes a caging or removable group, which is denoted as “CG” in the structure drawings. While a few examples are provided, it is understood that any appropriate convertible nucleobase structure that includes a photoremovable caging group may be used in accordance with the various embodiments. In FIGS. 5A-5B, both the unconverted state and converted state of these nucleobase structures are uniquely identifiable by current sequencing methods.

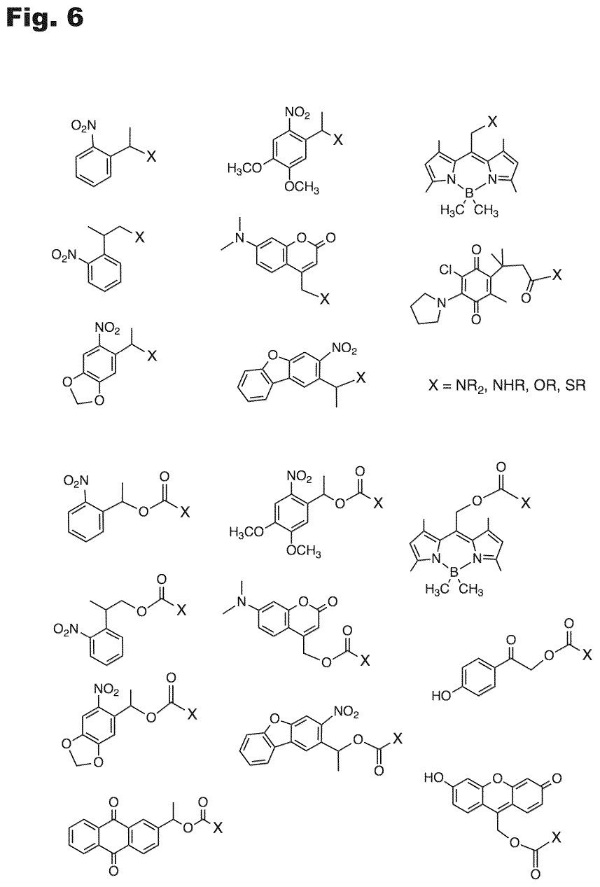

FIG. 6 provides further examples of photoremovable caging groups that can be utilized with the nucleobase structures to provide convertible nucleobases that can be converted from a first state into a second state by localized pulses of light. In various embodiments, any one of the photoremovable caging groups of FIG. 6 can be combined with the nucleobase structures in FIGS. 4 and 5A-5B. The photoremovable caging groups include a linker denoted as “X” which connect to the nucleobase structure denoted as R. In addition to the examples provided, various other photoremovable caging groups can be incorporated into light convertible nucleobases (see, e.g., D. D. Young and A. Deiters. Org Biomol Chem. 2007:5:999-1005; and Y. Wu. Z. Yang, and Y. Lu. Curr Opin Chem Biol. 2020:57:95-104; the disclosures of which are each incorporated herein by reference).

Numerous embodiments are also directed to a writable nucleic acid polymer further incorporating one or more of spacers, delimiters, and data tags. In accordance with various embodiments, a spacer is molecular residue incorporated within a writable nucleic acid polymer that provides a requisite space between convertible nucleobases in accordance with spatial resolution of the data writing mechanism. In many embodiments, a spacer will be distinguishable from convertible nucleobases such that when the data is read in a sequencer, the spacer does not interfere with the ability to read the convertible nucleobases. In some embodiments, a spacer is unreactive with the data writing mechanism. In some embodiments, a writable nucleic acid polymer will utilize the same residue repeatedly for each and every spacer. In some embodiments, however, a writable nucleic acid polymer will utilize two or more different residues as spacers. Any appropriate residue that is distinguishable from the convertible nucleobases may be utilized as spacers, including naturally occurring nucleobases, unnatural nucleobases, tetrahydrofuran abasic residues, and/or ethylene glycol residues.

In some embodiments, a spacer is distinguishable from convertible nucleobases and/or converted nucleobases such that when the data is read in a sequencer, the spacer does not interfere with the ability to encode data and decode/read the encoded data. In some embodiments, a spacer is unreactive with the data encoding mechanism.

A delimiter, in accordance with various embodiments, is a residue that signifies a boundary. In some embodiments, a delimiter is utilized to separate two adjacent data fields. Any appropriate residue that is distinguishable from the convertible nucleobases may be utilized as a delimiter, including naturally occurring nucleobases, unnatural nucleobases, tetrahydrofuran abasic residues, and/or ethylene glycol residues.

In several embodiments, a data tag is a string of residues (typically 4 or more residues) that signifies certain data. For instance, a data tag can signify type of data, date, data source, or any other information. Any appropriate residues that are distinguishable from the convertible nucleobases may be utilized as data tag residues, including naturally occurring nucleobases, unnatural nucleobases, tetrahydrofuran abasic residues, and/or ethylene glycol residues.

In another aspect, also provided herein are methods for generating a writable nucleic acid polymer, comprising providing a circular single-stranded oligonucleotide template, wherein the circular single-stranded oligonucleotide template is complementary to a repeating data field that comprises convertible nucleobases; and incubating the circular single-stranded oligonucleotide template in the presence of a nucleic acid primer, a polymerase, and triphosphate nucleotides, wherein the triphosphate nucleotides comprise convertible nucleobases in a first state and are capable of being converted from the first state into a second state, the first state and the second state being different.

In some embodiments, the circular single-stranded oligonucleotide template comprises nucleobases complementary to the convertible nucleobases, and wherein the complementary nucleobases are iteratively spaced such that the incubation of the template with the nucleic acid primer, the polymerase, and the triphosphate nucleotides provides a nucleic acid polymer comprising a plurality of the convertible nucleobases iteratively spaced along and covalently linked via the backbone of the nucleic acid polymer; wherein the plurality of the convertible nucleobases are covalently linked to the nucleic acid polymer in the first state and in the second state.

In some embodiments, the repeating data field further comprises spacer nucleobases, and wherein the triphosphate nucleotides further comprise triphosphate spacer nucleotides.

In another aspect, also provided herein are methods for generating a writable nucleic acid polymer, comprising chemically synthesizing a plurality of oligomers, each oligomer comprises a plurality of convertible nucleobases iteratively spaced along and linked via the nucleic acid polymer backbone, wherein each of the plurality of convertible nucleobases has a first state and is capable of being converted from the first state into a second state; wherein the plurality of convertible nucleobases are attached covalently to the nucleic acid polymer in the first state and in the second state, the first state and the second state being different; and ligating the plurality of oligomers to form the writable nucleic acid polymer.

In some embodiments, each of the plurality of oligomers comprises a plurality of spacer residues linked via the backbone of the nucleic acid polymer, wherein each of the plurality of the convertible nucleobases is separated by one or more spacer residues of the plurality of spacer residues. In some embodiments, the ligating step is via chemical ligation. In some embodiments, the ligating step is via enzymatic ligation. In some embodiments, a complementary DNA splint is used in the ligating step.

In some embodiments, the plurality of oligomers have the same sequence. In some embodiments, the plurality of oligomers are a plurality of copies of the same sequence. In some embodiments, the plurality of oligomers have different sequences.

In some embodiments, the method further comprising annealing a plurality of complements to the oligomers prior to the ligating step.

Writable nucleic acids can be generated by any appropriate method for generating long nucleic acid polymers. Generally, in accordance with various embodiments, polymerase extension or chemical synthesis is utilized to generate writable nucleic acid polymers. If polymerase extension is utilized, appropriate convertible nucleobases and residues that can be polymerized by the polymerase are to be utilized. If chemical synthesis is utilized, a broader range of convertible nucleobases and residues, but generally synthesis results in shorter nucleic acid strands (e.g., between 10 and 200 residues), which can be ligated together to generate longer nucleic acid polymers. It is understood that both polymerase and ligation methods can construct repeating writable polymers in either single-stranded or double-stranded states.

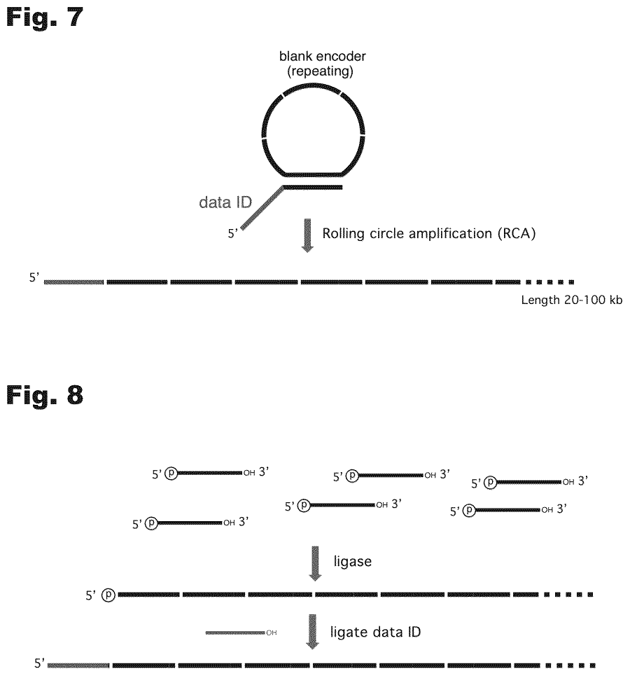

Illustrated in FIG. 7 is an example of generating a writable nucleic acid utilizing polymerase extension, and in particular, the figure illustrates an enzymatic rolling circle reaction method. In certain embodiments, a circular single-stranded DNA oligonucleotide is utilized as template (M. G. Mohsen and E. T. Kool, Acc Chem Res. 2016; 49: 2540-2550, the disclosure of which is incorporated herein by reference). The circular single-stranded DNA oligonucleotide is complementary to the repeating data field that comprises convertible nucleobases. In various embodiments, the circular single-stranded DNA oligonucleotide further comprises spacers, delimiters, and/or data tags. In various embodiments, the circular DNA size is 2-2000 nucleotides in length, preferably 2-200 nucleotides in length, and more preferably 45-95 nucleotides in length.

Once the nucleic acid circular template encoding the repeating data fields is constructed, it is incubated with a nucleic acid primer, a polymerase, a suitable buffer to support polymerase activity, and nucleoside triphosphates suitable for generating the writable nucleic acid. The primer binds the circle and the polymerase then produces a long repeating complement of the circle. Rolling circle nucleic acid synthesis is documented to proceed for many thousands of nucleotides, producing long DNA repeats (see M. M. Ali, et al., Chem Soc Rev. 2014:43:3324-41; and M. G. Mohsen and E. T. Kool. Acc Chem Res. 2016 Nov. 15; 49 (11): 2540-2550; the disclosures of which are incorporated herein by reference). In some embodiments, a data tag is utilized, which may be included at the remote 5′-end of the primer, and remains non-complementary to the DNA circle. Rolling circle DNA synthesis in this case will result in the repeating writable nucleic acid with a data tag attached to the 5′-end. If writable nucleic acid polymers are desired to be double-stranded, a primer complementary to the repeating data fields can be used together with a polymerase and nucleotides complementary to the first polymer to generate the complementary strand.

FIG. 8 illustrates a chemical synthesis and ligation method for generating a writable nucleic acid. In some instances, nucleotides for incorporation into a writable nucleic acid are not efficient polymerase substrates, especially many unnatural nucleobases, preventing the ability to effectively use a polymerase to generate long strands of the nucleic acid polymer. In a chemical synthesis and ligation approach, short writable nucleic acid polymers are constructed on a DNA synthesizer, which can be done utilizing phosphoramidite synthesis protocols, typically resulting in polymer lengths of 10-200 nucleotides. To assist in ligation, in some embodiments, the short-synthesized polymer further comprises a 5′-phosphate group and a native unaltered 3-hydroxyl group. A DNA ligase enzyme in the presence of ATP (e.g., T4 DNA ligase) will join the short polymers together to generate a long repeating polymer. In some embodiments, a complementary “splint” nucleic acid oligonucleotide that can hybridize to the reactive ends is utilized to assist ligation.

In some embodiments, to generate a double-stranded writable nucleic acid, a nucleic acid complement comprising a 5′-phosphate group is synthesized. Prior to ligation, the complement strand hybridizes with the writable nucleic acid. In some embodiments, hybridization of the complement strand results in a duplex with sticky ends that can be efficiently ligated into a double-stranded writable nucleic acid polymer utilizing a ligase enzyme.

Ligation-derived polymer molecules may result in a range of polymer lengths. In some embodiments, a mixture of polymers with variable lengths is used for data encoding. In some embodiments, a specific length is enriched and/or isolated (e.g., by electrophoresis) and subsequently used for data encoding.

Several embodiments are directed to polymerase expansion of writable nucleic acid polymers via repetitive expansion using a thermostable polymerase (e.g., DNA polymerase from Thermococcus litoralis). For more on polymerase expansion of repetitive regions, see J. S. Hartig and E. T. Kool. Nucleic Acids Res. 2005:33:4922-7, the disclosure of which is incorporated herein by reference.

If the ends of the data field DNA to be ligated are inefficient as a ligase enzyme substrate because of poor hybridization or an unnatural structure that interferes with the enzyme, in accordance with various embodiments, natural nucleobases can be added at ligation sites to ensure a good hybridization/ligation. In some embodiments, chemical ligation is utilized to generate a writable nucleic acid polymer. Chemical ligation can be achieved with cyanogen bromide, with carbodiimide reagents, or by nucleophilic reaction of a phosphorothioate group on one nucleic acid polymer strand terminus and a leaving group, such as (for example) iodide, on the other nucleic acid polymer strand terminus. Although chemical ligation involves joining of a phosphate end to a hydroxyl end, the reaction may be carried out with a 5″-phosphate and 3-hydroxyl, or a 3″-phosphate and a 5-hydroxyl. Such methods of chemical ligation have been described (see E. T. Kool, Acc Chem Res. 1998; 31:502-510; C. Obianyor, et al., Chembiochem. 2020; 21:3359-3370; and Y. Xu and E. T. Kool, Nucleic Acids Res. 1999:27:875-81: the disclosures of which are each incorporated herein by reference).

### Methods and Systems of Data Writing and Reading

In another aspect, provided herein are systems and methods for writing or reading the writable or written polymers provided herein (e.g., nucleic acid polymers).

### Systems

In another aspect, provided herein are systems for data writing, comprising: a writable polymer comprising a plurality of convertible residues iteratively spaced along and covalently linked to the backbone of the polymer, wherein each of the plurality of convertible residues has a first state and is capable of being converted from the first state into a second state, the first state and the second state being different and the plurality of convertible residues in the first state and the second state are readable by a polymerase enzyme; wherein the plurality of convertible residues are attached covalently linked to the polymer in the first state and in the second state; and a data writing device for writing data on the writable polymer.

In some embodiments, the writable polymer is a writable nucleic acid polymer and the plurality of convertible residues are convertible nucleobases. In some embodiments, the data writing device comprises a nanopore. In some embodiments, the data writing device converts the plurality of convertible nucleobases into the second state by light pulses, voltage pulses, an enzymatic agent, or a redox agent. In some embodiments, the data writing device converts the converts the plurality of convertible nucleobases into the second state by light pulses. In some embodiments, the data writing device comprises a light irradiation device.

### Methods for Writing Encoding Writable Polymers

In yet another aspect, provided herein are methods for writing data onto a writable polymer, comprising: providing a writable polymer that comprises a plurality of convertible residues iteratively spaced along and covalently linked via the backbone of the writable polymer, wherein each convertible residues of the plurality of convertible residues has a first state and is capable of being converted from the first state into a second state, the first state and the second state being different and the plurality of convertible residues in the first state and the second state are readable by a polymerase enzyme; and selectively converting, utilizing a data writing device, one or more of the plurality of convertible residues into the second state such that a data encoded polymer is generated.

Several embodiments are directed towards writing and reading data on nucleic acid polymers. In many embodiments, a writable nucleic acid polymer is provided having convertible nucleobases iteratively spaced along the writable polymer. The provided writable nucleic acid polymer may also have spacers, delimiters, and data tags, as described herein. To write data upon a nucleic acid polymer, in accordance with various embodiments, an individual strand is passed through a device having a nanopore. The device having a nanopore further provides a means for selectively converting a convertible nucleobase from a first state into a second state. A number of means can be utilized for converting a convertible nucleobase, including (but not limited to) light pulses, voltage pules, an enzymatic agent, a chemical reagent, and/or a redox agent. An example of a nanopore device for passing DNA through and encoded with localized light pulses is described within the examples provided in the Exemplary Embodiments.

In some embodiments, the writable polymer is a writable nucleic acid polymer and the plurality of convertible residues are convertible nucleobases. In some embodiments, the data writing device comprises a nanopore, and the method further comprising passing the writable polymer through the nanopore of the writing device, wherein the nanopore comprises converts one or more of the plurality of convertible residues into the second state.

In some embodiments, the nanopore is a plasmonic nanopore that provides localized excitation energy to selectively convert convertible nucleobases from the first state into the second state. In some embodiments, the data writing device comprises a plasmonic well or channel, and the method further comprising transferring the writable polymer into the plasmonic well or channel of the data encoding device, wherein the plasmonic well or channel provides local excitation from light pulses to selectively convert convertible nucleobases from the first state into the second state. In some embodiments, the data writing device selectively coverts the convertible residues into the second state by light pulses, voltage pulses, an enzymatic agent, or a redox agent. In some embodiments, the data writing device selectively converts the converts the convertible residues into the second state by light pulses.

In some embodiments, the convertible residues become naturally occurring nucleobases after conversion into the second state.

In some embodiments, the starting position and/or the ending positions of the writing on the writable polymer can be any position (i.e., any convertible residue such as convertible nucleobase) in the writable polymer (e.g., writable nucleic acid polymer) and specific starting and/or ending positions are not needed.

In some embodiments, the selectively converting step starts on either end of the writable polymer (e.g. the 5′ or 3′ end of a nucleic acid polymer). In some embodiment, the selectively converting step starts on the 5′ or the 3′ end of the nucleic acid polymer. In some embodiment, the selectively converting step selectively converts the convertible residues (e.g., convertible nucleobases) in either direction of the writable polymer. In some embodiments, the selectively converting step selectively converts the convertible nucleobases (e.g., writable bits) in either the 5′ to 3′ direction or the 3′ to 5′ direction. In some embodiment, the selectively converting step starts on the 5′ end of the nucleic acid polymer. In some embodiment, the selectively converting step starts on the 3′ end of the nucleic acid polymer.

In some embodiments, the writing starts at any position (e.g., any convertible residue such as convertible nucleobase) on the writable polymer. In some embodiments, the writing ends at any position (e.g., any convertible residue such as convertible nucleobase) on the writable polymer. In some embodiments, the writing starts and ends at any position (e.g., any convertible residue such as convertible nucleobase) on the writable polymer.

In some embodiments, the writable polymer is writable over its entire length, and the writing starts at the beginning position (e.g., the 3′ end of a nucleic acid polymer) and ends at the end position (e.g., the 5′ end of the nucleic acid polymer).

In some embodiments, the plurality of convertible residues comprise two or more types of convertible residues, wherein a first type of convertible residues are activatable by light of a first wavelength and a second type of convertible residues are activatable by light of a second wavelength. In some embodiments, the iterative spacing among the plurality of the convertible residues conforms to a resolution of the data writing device for selectively converting the convertible residues. In some embodiments, the selectively converting step does not require specific positioning of the writable polymer. In some embodiments, the conversion of the convertible residues into the second state is non-uniform on the data encoded polymer. In some embodiments, the conversion of the convertible residues into the second state is not limited to certain positions on the data encoded polymer.

In some embodiments, the writable polymer comprises a plurality of convertible residues regularly spaced along the writable polymer. In some embodiments, the data encoded polymer after the data is written comprises stochastically or irregularly spaced converted nucleobases.

In some embodiments, the plurality of convertible nucleobases are capable of being converted by light of a wavelength of 325 nm, 360 nm, or 400 nm.

In some embodiments, the plurality of convertible nucleobases are capable of being converted by light of a wavelength of between 400 nm to 850 nm.

In some embodiments, the method further comprises stretching or combing the writable polymer (e.g., a writable DNA) on a solid support.

In some embodiments, the method further comprises visualizing locations of the convertible residues using a dye.

In some embodiments, the method further comprises locally illuminating or locally exciting the writable polymer. In some embodiments, the locally illuminating or locally exciting uses Stimulated Emission Depletion (STED) laser.

In some embodiments, the method further comprises joining two or more data fields from two or more writable polymers end-to-end, resulting in a joined polymer comprising two or more data fields.

In some embodiments, the method further comprises controlling the passage rate of the writable polymer through the nanopore of the writing device.

In some embodiments, a plurality of writable polymers pass through the data writing device or multiple devices in parallel to write the same data (e.g., generating data redundancy).

In some embodiments, data encoded polymers generated by selectively converting convertible nucleobases comprises different polymer molecules encoded with the same data. In some embodiments, the data encoded nucleic acid polymers comprise converted nucleobases at different positions along the nucleic acid polymers (e.g., differently and optionally irregularly spaced) but encoding the same data (e.g., the sequential order of the written data bits are the same among different encoded polymer molecules).

In some embodiments, to encode data on a writable nucleic acid polymer provided herein, in accordance with various embodiments, an individual polymer has light energy or redox energy impinged upon the polymer in an iterative fashion such that it can controllably and selectively convert the convertible nucleobases to encode a data code (e.g., a binary data code).

Although a device with a nanopore is described, any device that can controllably and selectively convert the convertible nucleobases in accordance with a data code. In some embodiments, the device utilizes plasmonic channels or plasmonic wells for controllably and selectively converting the convertible nucleobases.

In several embodiments, as a writable nucleic acid polymer passes through the nanopore, the device selectively provides the means for converting the convertible nucleobase. For instance, if a nucleobase is to be converted into a second state via light pulses, as the nucleic acid polymer passes through the nanopore, the device can provide light such that it contacts the convertible nucleobase and converts the convertible nucleobase into the second state. If a nucleobase is to remain in a first state, the device will not provide light such that the convertible nucleobase will pass through the nanopore without conversion. In many embodiments, to ensure a device only converts a single nucleobase, the convertible nucleobase can be flanked with spacers in accordance with the device's writing resolution. For instance, if an optical light source and device with 1 nm of resolution is used to alter nucleobases, then each convertible base needs to be separated by at least 1 nm.

In certain embodiments, if a nucleobase is to be converted into a second state via light pulses, as the nucleic acid polymer passes through the nanopore, the device can provide light such that it only contacts the set of convertible nucleobases to be converted. If a nucleobase is to remain in the initial state, the device will not provide light such that the convertible nucleobase will pass through the nanopore without conversion. In many embodiments, to ensure a device only converts a set of nucleobase, the set of convertible nucleobases can be flanked with spacers in accordance with the device's writing resolution.

In some embodiments, to ensure a device only converts a single nucleobase (or a set of nucleobases), the device utilizes two or more means for converting a nucleobase: a first means being able to convert a first nucleobase structure but not a second nucleobase structure and a second means being able to convert the second nucleobase structure but not the first nucleobase structure. For instance, a device can utilize two wavelengths of light for providing energy such that the first wavelength is able to convert a first nucleobase structure but not a second nucleobase structure and a second wavelength is able to convert the second nucleobase structure but not the first nucleobase structure.

In some embodiments, to ensure a device only converts a single nucleobase (or a set of nucleobases), the device utilizes two or more means for converting a nucleobase: a first means being able to convert a first nucleobase structure but not a second nucleobase structure and a second means being able to convert both the first nucleobase structure and the second nucleobase structure concurrently as a pair. For instance, a device can utilize two wavelengths of light for providing energy such that the first wavelength is able to convert a first nucleobase structure but not a second nucleobase structure and a second wavelength is able to convert both the first nucleobase structure and the second nucleobase structure concurrently as a pair.

In many embodiments, the writing device is provided a code for writing the data into the nucleic acid polymer. Accordingly, the writing device will selectively convert various nucleobases of the polymer that are akin to being a “1” in binary code, while selectively allowing nucleobases of the polymer to pass through the pore without conversion that are akin to being a “0”. After writing a data code into the nucleic acid polymer, it can be stored by any appropriate means for storing nucleic acid molecules. For instance, data written nucleic acid polymers can be stored dry, as a precipitate, or in an appropriate nuclease-free solution at room temperature, or at colder temperatures (e.g., −20° C.). Stabilizers such as (for example) alcohol, chelating agents and nuclease inhibitors, may be included with the stored nucleic acid.

In some embodiments, the polymers provided herein (e.g., nucleic acid polymers) can be stored under standard nucleic acid storage protocols. In some embodiments, the polymer is a nucleic acid polymer that can be stored in appropriate nuclease-free solution at room temperature, or at a lower temperature (e.g., −20° C.). In some embodiments, the polymer can be stored at room temperature without stabilizer.

In many embodiments, the data encoding device is provided a code for writing the data into the nucleic acid polymer. Accordingly, in some embodiments, the encoding device will selectively convert various nucleobases of the polymer that in accordance with the code. In some embodiments that use solitary nucleobases as a bit, a data is encoded by selecting converting some of the nucleobase and selectively not converting the others, resulting in a binary code of converted and unconverted nucleobases. In some embodiments that use solitary nucleobases as a bit, a data is encoded by selectively converting some of the nucleobase into a first converted structure and selectively converting others into a second converted structure, resulting in a binary code of converted nucleobases: any unconverted nucleobases remain unencoded and are not utilized to decode the data code.

In some embodiments that utilize a set of nucleobases to encode a bit, each set will comprise at least two convertible nucleobases and the encoding device will selectively convert a first nucleobase of some of the sets into a converted structure and selectively convert a second nucleobase of other sets into a converted structure, resulting in a binary code. In some embodiments that utilize a set of nucleobases to encode a bit, each set will comprise at least two convertible nucleobases and the encoding device will selectively convert a first nucleobase of some of the sets into a converted structure and selectively convert both nucleobases of other sets into a converted structure, resulting in a binary code.

In some embodiments, nucleic acid polymers most efficiently store data at the single molecule level, providing the highest potential density of information. In some embodiments, however, if redundancy of data is required for better accuracy of data storage, then a plurality of nucleic acid polymers could be used to redundantly write the same data on each polymer of the plurality. Error correction algorithms are already well developed for digital data storage, and some of these algorithms can be applied in the present approach (see J. Li, et al., IEEE Transactions on Emerging Topics in Computing. 2021:9:651-663, the disclosure of which is incorporated herein by reference).

In various embodiments in which the encoded data is to be decoded by sequencing by synthesis (SBS), it may be desirable to have a redundancy of data and thus the same data on each polymer of the plurality. For instance, when using a nucleobase structure such as O6-nitrobenzyl-guanine, the structure is read as a mix of A and G using SBS and thus a redundancy of reading the structure would be needed to interpret whether the structure is O6-nitrobenzyl-guanine, guanine, or adenine. In some methods of SBS, the redundancy is inherent to each single sequence being read.

### Methods for Reading Decoding Writable Polymers

In another aspect, also provided herein are methods for reading data from a polymer encoded with data, comprising: providing the polymer encoded with data comprising convertible residues iteratively spaced along and covalently linked via the backbone of the polymer, wherein a first subset of the convertible residues are in a first state and a second subset of the convertible residues are in a second state, the first state and the second state being different and the plurality of convertible residues in the first state and the second state are readable by a polymerase enzyme; and passing the writable polymer encoded with data through a data reading device to read the encoded data on the polymer encoded with data.

In some embodiments, the writable polymer is a writable nucleic acid polymer and the plurality of convertible residues are convertible nucleobases. In some embodiments, the convertible residues in the first state can be converted into the second state via light. In some embodiments, the data reading device comprises a nanopore. In some embodiments, the data reading device is a sequencing device. In some embodiments, the sequencing device is a sequencing by synthesis device.

In some embodiments, the method further comprising measuring current flow of electrolytes during passage of the writable polymer.

In some embodiments, the method further comprising determining whether each of the plurality of convertible residues is in the first state or the second state based on the measured current flow of electrolytes during passage of the writable polymer.

In some embodiments, the method further comprising re-passing the polymer encoded with data through the data reading device to re-read the encoded data on the polymer encoded with data.

In some embodiments, the method further comprising validating and correcting the encoded data on the polymer encoded with data by comparing the encoded data on multiple copies of the polymer encoded with data.

In another aspect, also provided herein are methods for reading or decoding data from a nucleic acid polymer encoded with data, the method comprising:
providing a plurality of redundant copies of the nucleic acid polymer encoded with data comprising:
a plurality of converted nucleobases, wherein each converted nucleobase comprises a first nucleobase structure, wherein the first converted nucleobase has been converted from a first state into a second state, the first state and the second state being different; and
a plurality of convertible nucleobases, wherein each convertible nucleobase comprising a second nucleobase structure and a directly linked removable group, and wherein the convertible nucleobase is provided in a first state and is capable of being converted from the first state into a second state by releasing the second removable group from the second nucleobase structure, the first state and the second state being different:
wherein the converted nucleobases and convertible nucleobases are linked via the nucleic acid polymer backbone; and
sequencing each redundant copy of the plurality redundant copies of the nucleic acid polymer.

In some embodiments, the method further comprising detecting the plurality of converted nucleobases and the plurality of convertible nucleobases; and decoding the data based on the detected plurality of converted nucleobases.

In some embodiments, the plurality of converted nucleobases in the first state and the second state are readable by a polymerase enzyme. In some embodiments, the plurality of convertible nucleobases in the first state and the second state are readable by a polymerase enzyme. In some embodiments, the plurality of converted nucleobases and the plurality of convertible nucleobases are detected based on the sequencing result of the redundant copies of the nucleic acid polymer encoded with data.

In some embodiments, the sequencing starts on either end of the writable polymer (e.g. the 5′ or 3′ end of a nucleic acid polymer). In some embodiment, the sequencing starts on the 5′ or the 3′ end of the nucleic acid polymer. In some embodiment, the sequencing starts on the 5′ end of the nucleic acid polymer. In some embodiment, the sequencing starts on the 3′ end of the nucleic acid polymer

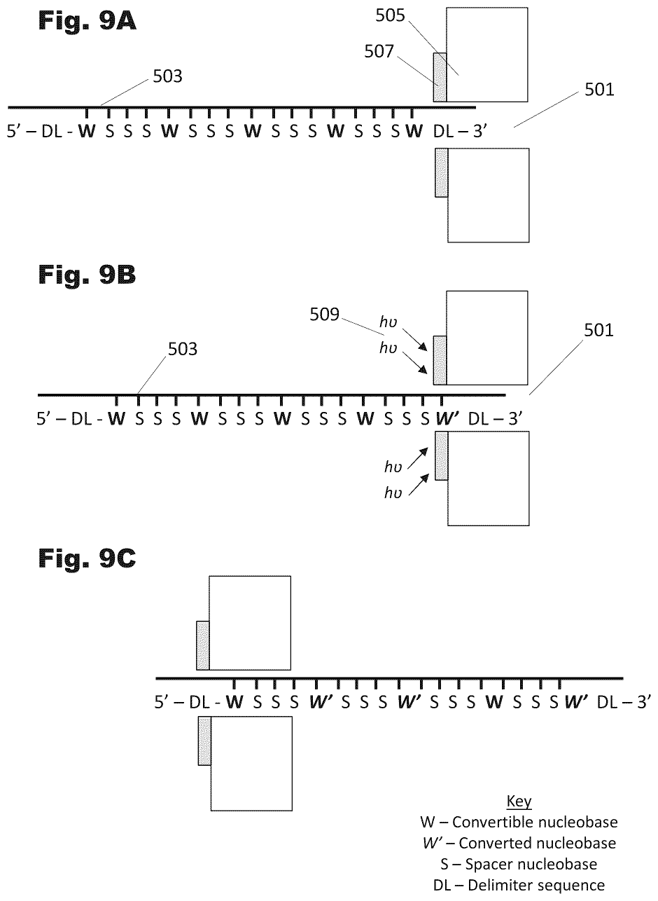

FIGS. 9A-9C illustrate an example of utilizing a device with a nanopore 501 for writing data into writable nucleic acid polymers 503. The device comprises a substrate 505 that includes a plasmonic nanostructure 507 for providing localized light energy to the writable polymer 503. The writable polymer 503 is controllably passed through a nanopore 501 at a steady rate. The nanopore may be comprised of protein or may be artificial, such as a pore engineered in silicon or other inorganic solid (see N Kono and K. Arakawa. Dev Growth Differ. 2019; 61:316-326; and Q Chen and Z. Liu. Sensors (Basel). 2019; 19:1886; the disclosures of which are each incorporated herein by reference). Methods for constructing nanopores, and methods for a controlled rate of passage, have been previously described (see Y. Zhishan, et al., Nanoscale Res Lett. 2020; 15: 80, the disclosure of which is incorporated herein by reference). As the writable nucleic acid polymer 503 passes through the nanopore 501 at the controlled rate, the device selectively converts individual convertible nucleobases as they pass through the pore as encoded. As shown in FIG. 9B, a pulse of light 509 can be impinged on the convertible nucleobase via a plasmonic nanostructure 507 locally just as it passes through the pore, which can be appropriately timed due to the controlled rate of passage through the pore. As result of selective nucleobase conversion, binary digital data is encoded into the polymer (FIG. 9C).

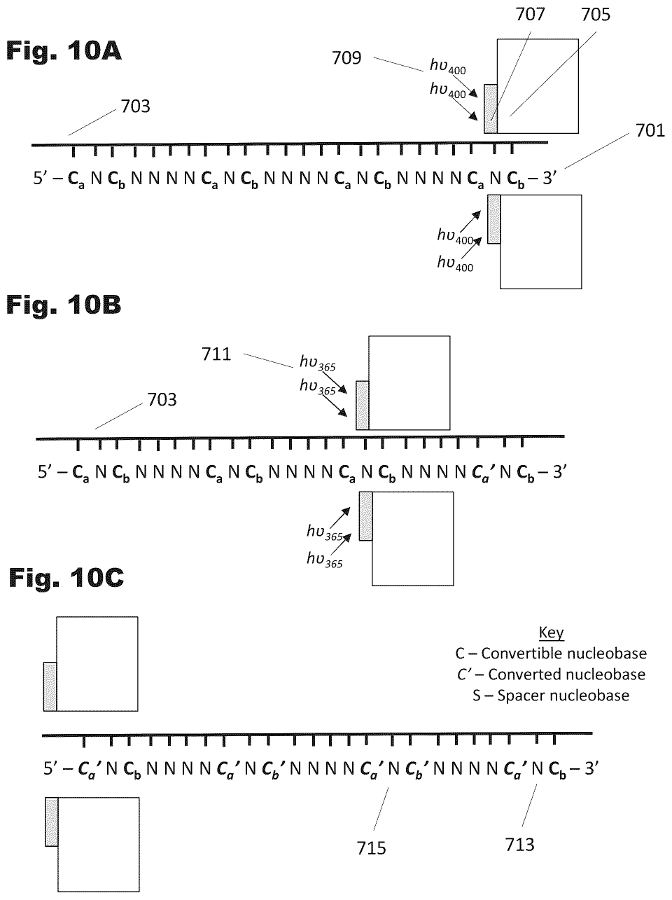

FIGS. 10A-10C illustrate another example of utilizing a device with a nanopore 701 for encoding data into encodable nucleic acid polymers 703 comprising a plurality of sets of convertible nucleobases that are iteratively repeated along the polymer. The device comprises a substrate 705 that includes a plasmonic nanostructure 707 for providing localized light energy of multiple wavelengths to the data encodable polymer 703. The polymer 703 is controllably passed through a nanopore 701 at a steady rate. As the data encodable nucleic acid polymer 703 passes through the nanopore 701 at the controlled rate, the device selectively converts one or both convertible nucleobases of each set as the set passes through the pore, as prescribed by a data code. In this example, the data code to be encoded is 1001, where 1 is represented by Cₐ′ and 0 is represented by Cₐ′C_{b}′. As shown in FIG. 10A, a pulse of light 709 at a first wavelength (e.g., 400 nm) can be impinged on a set via a plasmonic nanostructure 707 locally just as it passes through the pore, which results in conversion of a single convertible base (as shown it converts base Cₐ into Cₐ′). As shown in FIG. 10B, a pulse of light 711 at a second wavelength (e.g., 365 nm) can be impinged on the set via a plasmonic nanostructure 707 locally just as it passes through the pore, which results in conversion of a both convertible bases (as shown it converts bases Cₐ and C_{b} into Cₐ′ and C_{b}′). As result of selective nucleobase conversion, binary digital data is encoded into the polymer 703, which is encoded via sets with single nucleobase conversion 713 and sets of dual nucleobase conversion 715 (FIG. 10C).

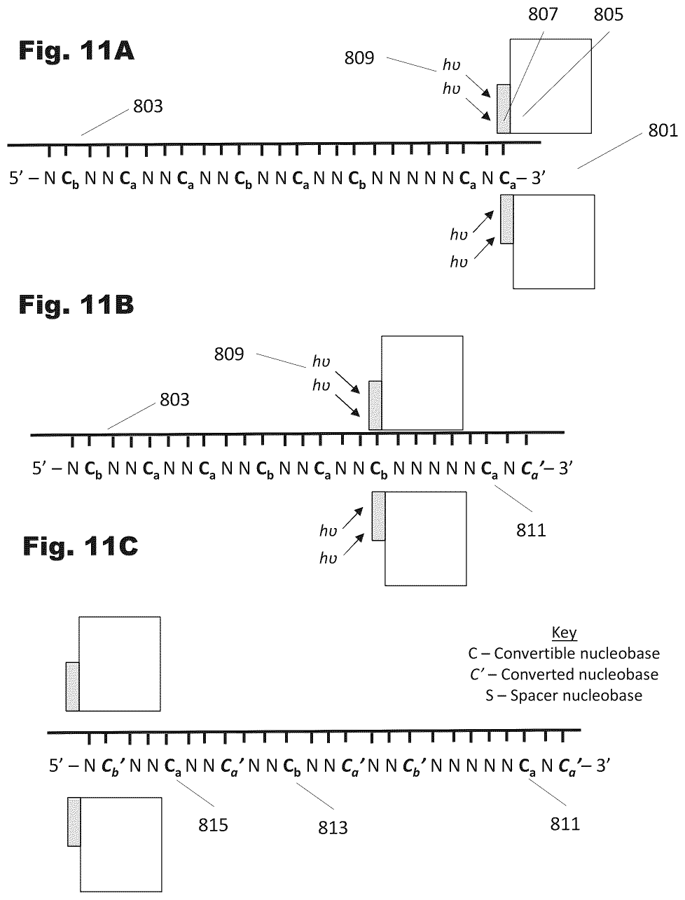

FIGS. 11A-11C illustrate yet another example of utilizing a device with a nanopore 801 for encoding data into encodable nucleic acid polymers 803 comprising a plurality of two convertible nucleobase structures that are stochastically or irregularly repeated along the polymer. The device comprises a substrate 805 that includes a plasmonic nanostructure 807 for providing localized light energy of one or more wavelengths to the data encodable polymer 803. The polymer 803 is controllably passed through a nanopore 801 at a steady rate. As the data encodable nucleic acid polymer 803 passes through the nanopore 801 at the controlled rate, the device selectively converts one convertible nucleobase structure at a time, as prescribed by a data code. In this example, the data code to be encoded is 10110, where 1 is represented by Cₐ′ and 0) is represented by C_{b}′. As shown in FIG. 11A, a pulse of light 809 can be impinged on a first nucleobase structure via a plasmonic nanostructure 807 locally just as it passes through the pore, which results in conversion of the nucleobase (as shown it converts base Cₐ into Cₐ′). As shown in FIG. 11B, a pulse of light 809 can be impinged on a second nucleobase structure via a plasmonic nanostructure 807 locally just as it passes through the pore, which results in conversion of the nucleobase (as shown it converts base C_{b} into C_{b}′). Further, as shown in FIGS. 11B and 11C, convertible bases 813, 815, and 817 are skipped, in accordance with the code. As result of selective nucleobase conversion, binary digital data is encoded into the polymer 803, which is encoded by converted nucleobases Ca′Cb′Ca′Ca′Cb′ and skipping any convertible base in accordance with the data code.

Highly localized light excitation can be achieved via specialized sub-wavelength microscopic focusing strategies such as STEDX, or by the use of nanoplasmonic structures such as bow ties or by the use of zero-mode waveguides (see Y. Fang and M Sun. Light Sci Appl. 2015; 4:e294; and X. Shi, et al. Small. 2018; 14: e1703307; the disclosures of which are each incorporated herein by reference). If redox is to be used for nucleobase conversion, an applied potential of an electrode near or in a nanopore or nanochannel can be used. With a regular rate of passage, timed electronic pulses of voltage potential can result in appropriate spacing of nucleobase conversion. For enzymatic nucleobase conversion, the writable nucleic acid polymer can be passed through two adjacent nanopores at a controlled rate; as a convertible nucleobase enters the volume between two pores, the enzyme is contacted (e.g. by microfluidics) with the strand at a local moiety/base/bit. Timing of microfluidic flow and controlled passage of the writable polymer can be in concert with appropriate spacing such that data is encoded with fidelity.

Several embodiments are also directed towards positive bit writing with dual bits. Accordingly, in certain embodiments, a writable nucleic acid polymer includes one or more repeated duads of convertible nucleobases, each convertible base of the duad is within the same field of resolution of the writing mechanism. In some embodiments, each convertible nucleobase of a duad is adjacent with other nucleobase of the duad. In some embodiments, each convertible nucleobase of a duad is near enough to the other nucleobase of the duad to be addressed in the same converting signal. In some embodiments, one convertible nucleobase of a duad has different reaction condition for nucleobase conversion than the other nucleobase of the duad. For example, in some embodiments, a first convertible nucleobase of a duad is converted by light at a first wavelength and a second convertible nucleobase of the duad is converted by light at a second wavelength. Thus, in certain embodiments of encoding a writable nucleic acid polymer comprising one or more duads, as each duad enters a nanopore, a particular reaction condition is provided to convert a first convertible nucleobase, or a second convertible nucleobase, or both the first and the second convertible nucleobases in accordance with a code.

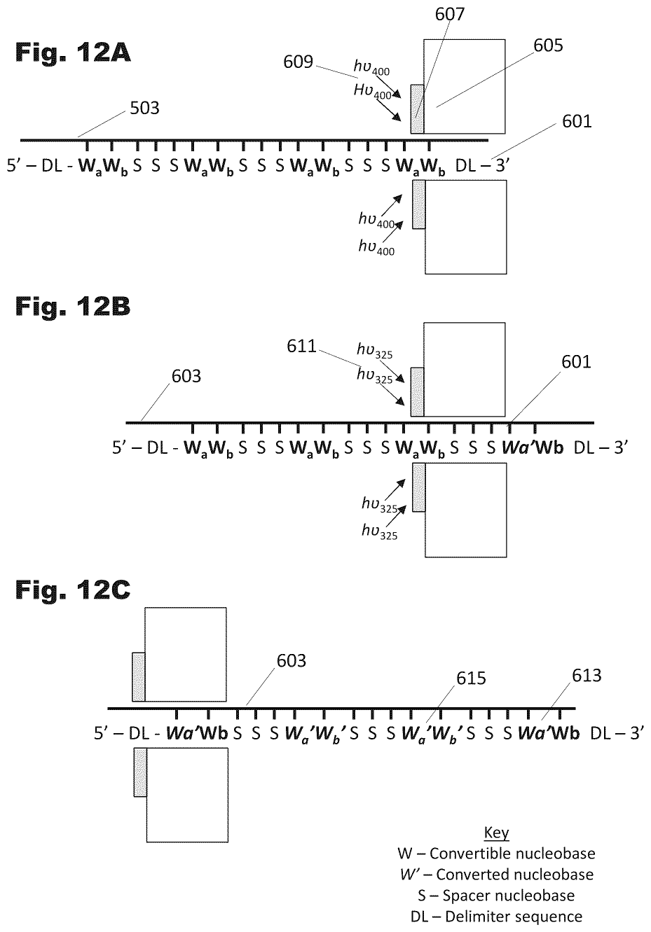

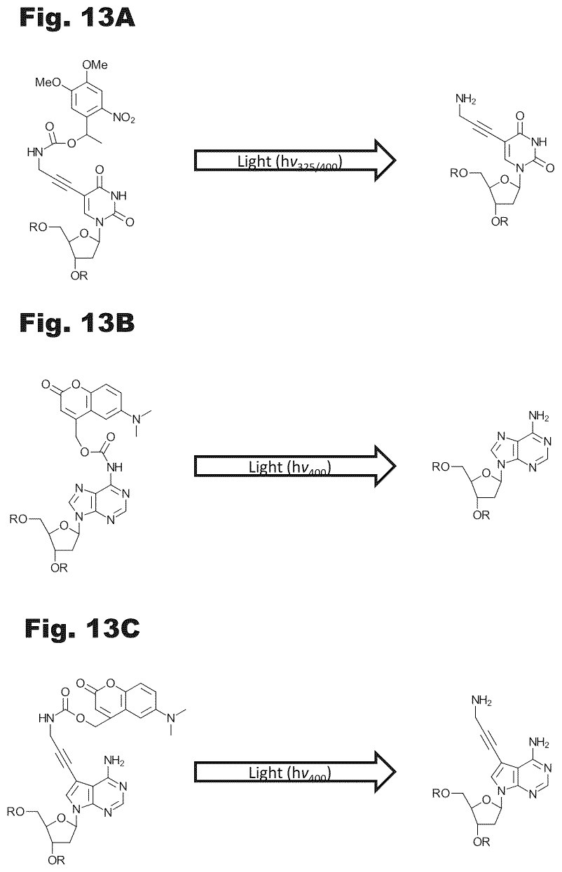

FIGS. 12A-12C illustrate an example of utilizing a device with a nanopore 601 for writing data into writable nucleic acid polymers 603 comprising a plurality of duads. The device comprises a substrate 605 that includes a plasmonic nanostructure 607 for providing localized light energy of multiple wavelengths to the writable polymer 603. The writable polymer 603 is controllably passed through a nanopore 601 at a steady rate. As the writable nucleic acid polymer 603 passes through the nanopore 601 at the controlled rate, the device selectively converts individual convertible nucleobases of a duad as the duad pass through the pore as encoded. As shown in FIG. 12A, a pulse of light 609 at a first wavelength (e.g., 400 nm) can be impinged on the duad via a plasmonic nanostructure 607 locally as it passes through the pore, which results in conversion of a single convertible base (as shown it converts base Wₐ into Wₐ′). As shown in FIG. 12B, a pulse of light 611 at a second wavelength (e.g., 325 nm) can be impinged on the duad via a plasmonic nanostructure 607 locally as it passes through the pore, which results in conversion of a both convertible bases (as shown it converts bases Wₐ and W_{b} into Wₐ′ and W_{b}′). As result of selective nucleobase conversion, binary digital data is encoded into the polymer 603, which is encoded via duads with single nucleobase conversion 613 and duads of dual nucleobase conversion 615 (FIG. 12C). Examples of convertible nucleobases that are converted at specific wavelengths are provided in FIGS. 13A-13C.

In many embodiments, to read the data on written nucleic acid polymers, any appropriate sequencer capable of reading unnatural and/or altered nucleobases can be utilized. In certain embodiments, a device is capable of writing and reading nucleic acid polymers. In certain embodiments, a nanopore has dual functionality for both writing and reading nucleic acid polymers, however, some devices may include distinct nanopores for performing writing and reading. Examples of commercial nanopore sequencers include Oxford Nanopore Technologies PromethION, MinION, and GridION sequencing platforms (Oxford, UK) and Pacific Bioscience's Single Molecule, Real-Time (SMRT) sequencing platform (Menlo Park, CA). Alternatively, a nanopore device can be fabricated or manufactured for writing and/or reading the data. The nanopore can be comprised of solid-state materials, or can contain one or more proteins.

In many embodiments, to decode the data on encoded nucleic acid polymers, any appropriate sequencer capable of reading unnatural and/or altered nucleobases can be utilized. Examples of sequencing techniques used to decode DNA include (but are not limited to) shotgun sequencing, long-read sequencing, nanopore sequencing, and sequencing by synthesis.

In various embodiments in which the encoded data is to be decoded by sequencing by synthesis (SBS), it may be desirable to have a redundancy of data and thus the same data on each polymer of the plurality. For instance, when using a nucleobase structure such as O6-nitrobenzyl-guanine, the structure is read as a mix of A and G using SBS and thus a redundancy of reading the structure would be needed to interpret whether the structure is O6-nitrobenzyl-guanine, guanine, or adenine.

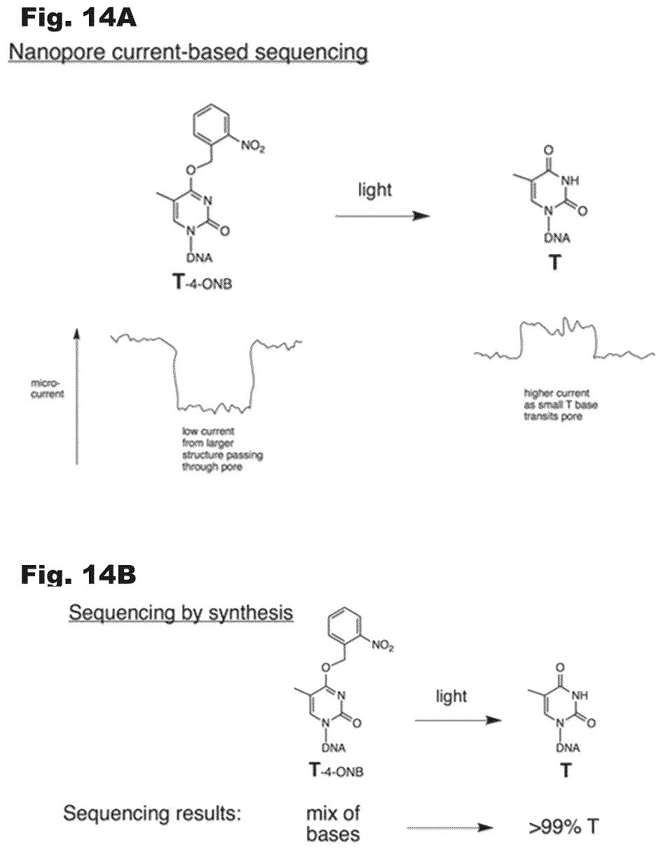

Provided in FIG. 14A is an example of utilizing a nanopore to read nucleobase sequences of convertible and converted nucleobases. In this example, O4-nitrobenzylthymine (T-4-ONB) is provided as the convertible base and removal of the nitrobenzyl group converts the nucleobases into a thymine. The current reading providing is differentiable between these two structures, as the microcurrent of T-4-ONB has low current and thymine has larger current. Although T-4-ONB is provided in this example, any convertible nucleobases in which an appreciable change in structure size and/or charge can be utilized, including (but not limited to) structures provided in FIGS. 4 and 5A-5B.

In certain embodiments, sequencing by synthesis (SBS) is performed to decode the data within a nucleic acid polymer, which may help in decoding between certain bases that have been converted and/or left unconverted. Standard SBS utilizes a polymerase a to read a strand of the DNA sequence and make a complementary copy of the strand. The converted nucleobases should have the ability to serve as polymerase substrates and yield a predictable sequence result, enabling the polymerase to incorporate a base opposite and continue in the synthesis. For example, O6-nitrobenzylguanine (O6NBG) is contemplated as a convertible base, which is a suitable substrate for a DNA polymerase enzyme, thus enabling its reading by SBS. Sequencing of O6NBG nucleobase yields a reading that is a mixture of A and G nucleobases encoded at that position (see, e.g., A. M. Kietrys, W. A. Velema, and E. T. Kool, J Am Chem Soc. 2017; 139:17074-17081, the disclosure of which is incorporated herein by reference). When the nitrobenzyl group is removed to convert into a guanine structure, however, the sequencing reads will have a clear signal of G. When utilizing SBS, sequencing of multiple copies of encoded nucleic acid can help differentiate whether a nucleobase is a converted structure (e.g., guanine) or an unconverted structure (e.g., O6-nitrobenzylguanine) at a given position, thus indicating the presence of whether data has been encoded at that position. Notably, sequencing of multiple copies of encoded nucleic acid may be helpful in distinguishing several convertible/converted nucleobase structures, such as the structures provided in FIGS. 4 and 5A-5B.

Provided in FIG. 14B is an example of utilizing SBS to read nucleobase sequences of convertible and converted nucleobases. In this example, O4-nitrobenzylthymine (T-4-ONB) is provided as the convertible base and removal of the nitrobenzyl group converts the nucleobases into a thymine. SBS of T-4-ONB results in reading of a mixture of bases whereas the removal of the nitrobenzyl group results in a specific reading of thymine (see, e.g., A. M. Kietrys, W. A. Velema, and E. T. Kool, J Am Chem Soc. 2017; 139:17074-17081, the disclosure of which is incorporated herein by reference). Although T-4-ONB is provided in this example, any convertible nucleobases in which the sequencing readings changes as a result of the conversion can be utilized, including (but not limited to) structures provided in FIGS. 4 and 5A-5B.

### Certain Embodiments

Embodiment 1. A nucleic acid polymer for encoding data, comprising:
a plurality of pairs of convertible nucleobases, wherein the pairs are iteratively spaced along the nucleic acid polymer and each convertible nucleobase is linked via the nucleic acid polymer backbone,
wherein each convertible nucleobase of each pair comprises a nucleobase structure and a leaving group, the leaving group linked to the nucleobase structure via a linker, and wherein each convertible nucleobase of each pair is provided in a first state and is capable of being converted from the first state into a second state by light energy or redox energy that releases the leaving group from the nucleobase structure.
Embodiment 2. The nucleic acid polymer of Embodiment 1 further comprising a first plurality of sets of spacer residues, each spacer residue linked via the nucleic acid polymer backbone, wherein each set of the first plurality comprises two or more spacer residues, wherein each set of the first plurality is provided in-between each pair of the plurality of pairs of convertible nucleobases to provide the iterative spacing among the plurality of pairs of convertible nucleobases.
Embodiment 3. The nucleic acid polymer of Embodiment 2 further comprising a second plurality of sets of spacer residues, each spacer residue linked via the nucleic acid polymer backbone, wherein each set of the second plurality comprises one or more spacer residues, wherein each set of second plurality is provided in-between the convertible nucleobases in each pair of nucleobases, and wherein the number of spacer residues in each set of the second plurality is less than the number of spacer residues in each set of the first plurality.
Embodiment 4. The nucleic acid polymer of Embodiment 1 or 2., wherein the iterative spacing among the pairs of convertible nucleobases is equal to or greater than a resolution of a data encoding mechanism for encoding data into the nucleic acid polymer.
Embodiment 5. The nucleic acid polymer of any one of Embodiments 1-4, wherein each convertible nucleobase comprises one of the following nucleobase structures: O6-guanine, N2-guanine, N7-guanine, N6-adenine, N5-adenine, O4-thymine, N3-thymine, 2-thio-thymine, 4-thio-thymine, N4-cytosine, or N3-cytosine.
Embodiment 6. The nucleic acid polymer of any one of Embodiments 1-5, wherein the leaving group comprises one of:

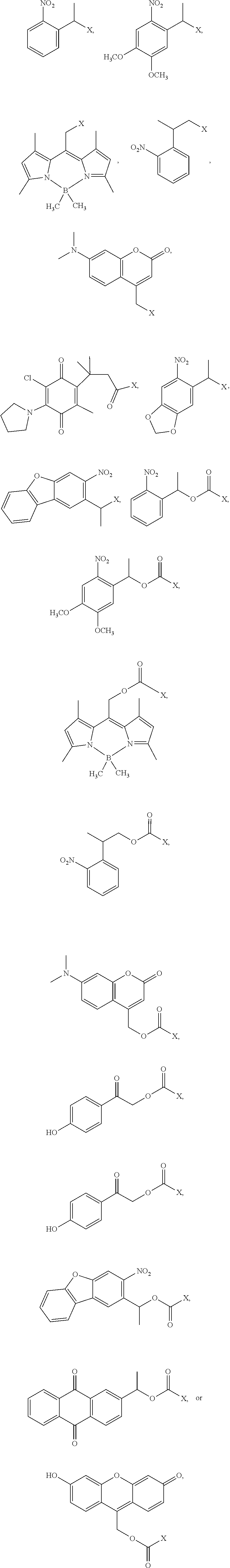

wherein X is the linker to the nucleobase structure, wherein the linker is one of: NR2, NHR, OR, or SR, and wherein R is the nucleobase structure.
Embodiment 7. The nucleic acid polymer of Embodiment 1, wherein light energy is used to release each leaving group, and wherein a first wavelength of light provides energy capable of converting a first convertible nucleobase of each pair into its second state, and wherein a second wavelength of light provides energy capable of converting a second convertible base of each pair into its second state.
Embodiment 8. The nucleic acid polymer of Embodiment 7, wherein the second wavelength of light provides energy that is further capable of converting the first convertible nucleobase of each pair into its second state.
Embodiment 9. A nucleic acid polymer for encoding data, comprising:
a first plurality convertible nucleobases stochastically or irregularly spaced along the nucleic acid polymer and linked via the nucleic acid polymer backbone, wherein each convertible nucleobase of the first plurality comprises a first nucleobase structure and a first leaving group, the first leaving group linked to the first nucleobase structure via a first linker, and wherein each convertible nucleobase of the first plurality is provided in a first state and is capable of being converted from the first state into a second state by light energy or redox energy that releases the first leaving group from the first nucleobase structure; and
a second plurality of convertible nucleobases stochastically or irregularly spaced along the nucleic acid polymer and linked via the nucleic acid polymer backbone, wherein each convertible nucleobase of the second plurality comprises a second nucleobase structure and a second leaving group, the second leaving group linked to the second nucleobase structure via a second linker, and wherein each convertible nucleobase of the first plurality is provided in a first state and is capable of being converted from the first state into a second state by light energy or redox energy that releases the second leaving group from the second nucleobase structure.
Embodiment 10. The nucleic acid polymer of Embodiment 9 further comprising a plurality of spacer residues linked via the nucleic acid polymer backbone, wherein spacer residues are stochastically or irregularly provided in between the convertible nucleobases.
Embodiment 11. The nucleic acid polymer of Embodiment 9 or 10, wherein each convertible nucleobase comprises one of the following nucleobase structures: O6-guanine, N2-guanine, N7-guanine, N6-adenine, N5-adenine, O4-thymine, N3-thymine, 2-thio-thymine, 4-thio-thymine, N4-cytosine, or N3-cytosine.
Embodiment 12. The nucleic acid polymer of any one of Embodiments 9-11, wherein the leaving group comprises one of:

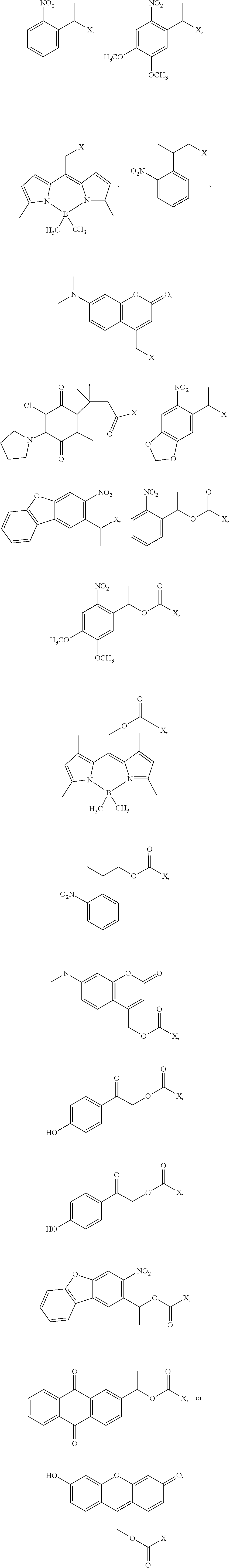

wherein X is the linker to the nucleobase structure, wherein the linker is one of: NR2, NHR, OR, or SR, and wherein R is the nucleobase structure.
Embodiment 13. A convertible nucleobase for use in a data encodable polymer, comprising: a nucleobase structure and a leaving group, wherein the leaving group is linked to the nucleobase structure via a linker, and wherein the leaving group is capable of being removed from the nucleobase structure by light energy or redox energy.
Embodiment 14. The convertible nucleobase of Embodiment 13, wherein the nucleobase structure comprises O6-guanine, N2-guanine, N7-guanine, N6-adenine, N5-adenine, O4-thymine, N3-thymine, 2-thio-thymine, 4-thio-thymine, N4-cytosine, or N3-cytosine.
Embodiment 15. The convertible nucleobase of Embodiment 13, wherein the leaving group comprises:

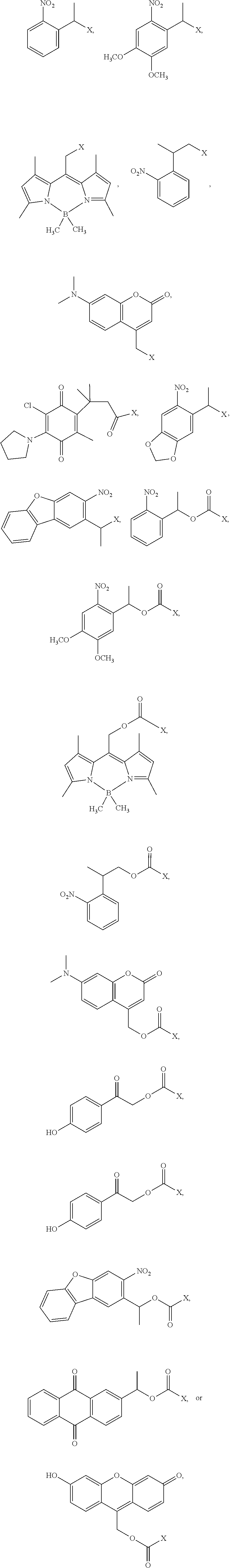

wherein X is a linker to the nucleobase structure, wherein the linker is one of: NR2, NHR, OR, or SR, and wherein R is the nucleobase structure.
Embodiment 16. The convertible nucleobase of Embodiment 15, wherein the linker comprises: NR2, NHR, OR, or SR, and wherein R is the nucleobase structure.
Embodiment 17. A data encoded nucleic acid polymer, comprising:
a plurality of pairs of nucleobases, wherein each pair of nucleobases comprises at least a first converted nucleobase, wherein the first converted nucleobase comprises a first nucleobase structure, wherein the first converted nucleobase has been converted from a first state into a second state by light energy or redox energy that released a first leaving group from the first nucleobase structure;
wherein each pair of nucleobases further comprises at least one of:
   a convertible nucleobase that comprises a nucleobase structure and a second leaving group, the second leaving group linked to the second nucleobase structure via a linker, and wherein the convertible nucleobase is provided in a first state and is capable of being converted from the first state into a second state by light energy or redox energy that releases the second leaving group from the second nucleobase structure; or
   a second converted nucleobase, wherein the second converted nucleobase comprises a second nucleobase structure, wherein the second converted nucleobase has been converted from a first state into a second state by light energy or redox energy that released a second leaving group from the second nucleobase structure; wherein the pairs of nucleobases are iteratively spaced along the nucleic acid polymer and the nucleobases are linked via the nucleic acid polymer backbone.
Embodiment 18. The nucleic acid polymer of Embodiment 17 further comprising a first plurality of sets of spacer residues, each spacer residue linked via the nucleic acid polymer backbone, wherein each set of the first plurality comprises two or more spacer residues, wherein each set of the first plurality is provided in-between each pair of the plurality of pairs of nucleobases to provide the iterative spacing among the plurality of pairs of nucleobases.
Embodiment 19. The nucleic acid polymer of Embodiment 18 further comprising a second plurality of sets of spacer residues, each spacer residue linked via the nucleic acid polymer backbone, wherein each set of the second plurality comprises one or more spacer residues, wherein each set of second plurality is provided in-between the convertible nucleobases in each pair of nucleobases, and wherein the number of spacer residues in each set of the second plurality is less than the number of spacer residues in each set of the first plurality.
Embodiment 20. The nucleic acid polymer of Embodiment 17 or 18, wherein the iterative spacing among the pairs of nucleobases is equal to or greater than a resolution of a data encoding mechanism used to encode data into the data encoded nucleic acid polymer.
Embodiment 21. The nucleic acid polymer of any one of Embodiments 14-20, wherein each converted nucleobase has one following nucleobase structures: guanine, adenine, thymine, or cytosine.
Embodiment 22. The nucleic acid polymer of any one of Embodiments 14-21, wherein each convertible nucleobase comprises one of the following nucleobase structures: O6-guanine, N2-guanine, N7-guanine, N6-adenine, N5-adenine, O4-thymine, N3-thymine, 2-thio-thymine, 4-thio-thymine, N4-cytosine, or N3-cytosine.
Embodiment 23. The nucleic acid polymer of any one of Embodiments 14-22, wherein the second leaving group of each convertible nucleobase comprises one of:

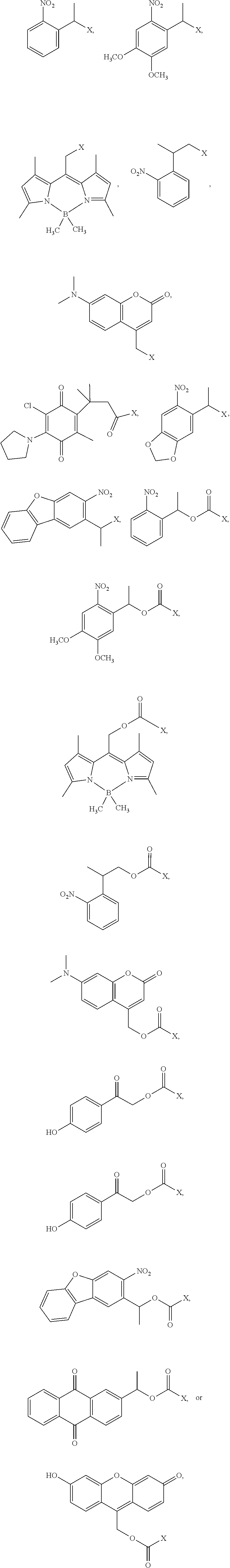

wherein X is a linker to the nucleobase structure, wherein the linker is one of: NR₂, NHR, OR, or SR, and wherein R is the nucleobase structure.
Embodiment 24. A data encoded nucleic acid polymer, comprising:
a first plurality of converted nucleobases stochastically or irregularly spaced along the nucleic acid polymer and linked via a nucleic acid polymer backbone, wherein each converted nucleobase of the first plurality comprises a first nucleobase structure, wherein each converted nucleobase of the first plurality has been converted from a first state into a second state by light energy or redox energy that released a first leaving group from the first nucleobase structure; and
a second plurality of converted nucleobases stochastically or irregularly spaced along the nucleic acid polymer and linked via a nucleic acid polymer backbone, wherein each converted nucleobase of the second plurality comprises a second nucleobase structure, wherein each converted nucleobase of the second plurality has been converted from a first state into a second state by light energy or redox energy that released a second leaving group from the second nucleobase structure.
Embodiment 25. The data encoded nucleic acid polymer of Embodiment 24, further comprising:
a first plurality of convertible nucleobases stochastically or irregularly spaced along the nucleic acid polymer and linked via the nucleic acid polymer backbone, wherein each convertible nucleobase of the first plurality comprises the first nucleobase structure and the first leaving group, wherein the first leaving group is linked to the first nucleobase structure via a first linker; and
a second plurality of convertible nucleobases stochastically or irregularly spaced along the nucleic acid polymer and linked via the nucleic acid polymer backbone, wherein each convertible nucleobase of the second plurality comprises the second nucleobase structure and the second leaving group, wherein the second leaving group is linked to the second nucleobase structure via a second linker.
Embodiment 26. The nucleic acid polymer of Embodiment 25 further comprising a plurality of spacer residues linked via the nucleic acid polymer backbone, wherein spacer residues are stochastically or irregularly provided in between nucleobases comprising the converted and convertible nucleobases.
Embodiment 27. The nucleic acid polymer of any one of Embodiment 24 to 26, wherein each converted nucleobase has one following nucleobase structures: guanine, adenine, thymine, or cytosine.
Embodiment 28. The nucleic acid polymer of any one of Embodiments 25 to 27, wherein each convertible nucleobase comprises one of the following nucleobase structures: O6-guanine, N2-guanine, N7-guanine, N6-adenine, N5-adenine, O4-thymine, N3-thymine, 2-thio-thymine, 4-thio-thymine, N4-cytosine, or N3-cytosine.
Embodiment 29. The nucleic acid polymer of any one of Embodiments 25 to 28, wherein the leaving group of each convertible nucleobase comprises one of

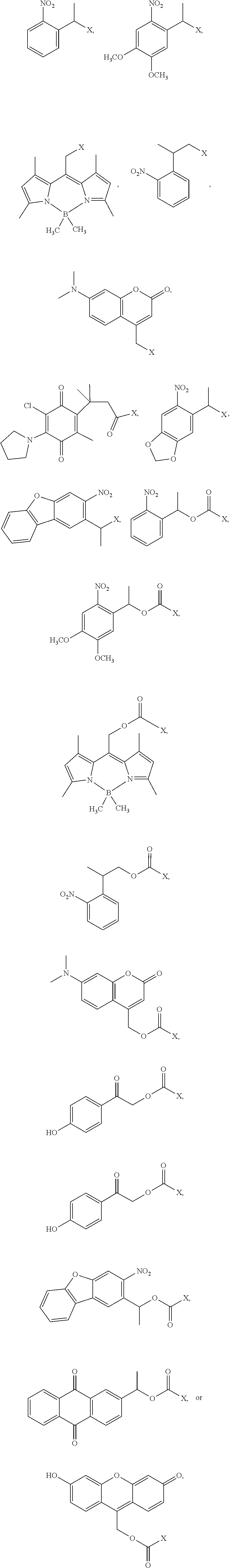

wherein X is a linker to the nucleobase structure, wherein the linker is one of: NR2, NHR, OR, or SR, and wherein R is the nucleobase structure.
Embodiment 30. A method of encoding data onto a data encodable nucleic acid polymer, comprising:
providing a data encodable nucleic acid polymer that comprises: a plurality of pairs of convertible nucleobases, wherein the pairs are iteratively spaced along the nucleic acid polymer and each convertible nucleobase is linked via the nucleic acid polymer backbone,
wherein each convertible nucleobase of each pair comprises a nucleobase structure and a leaving group, the leaving group linked to the nucleobase structure via a linker, and wherein each convertible nucleobase of each pair is provided in a first state and is capable of being converted from the first state into a second state by light energy or redox energy that releases the leaving group from the nucleobase structure; and
selectively converting, utilizing a data encoding device, at least one nucleobase of each pair of convertible nucleobases into the second state by providing a light energy or redox energy to release the leaving group from the nucleobase structure of the at least one nucleobase.
Embodiment 31. The method of Embodiment 30, wherein the data encoding device comprises a plasmonic nanopore, and the method further comprising: passing the data encodable nucleic acid polymer through the plasmonic nanopore of the data encoding device, wherein the plasmonic nanopore provides the light energy or redox energy to release the leaving group from the nucleobase structure of the at least one nucleobase.
Embodiment 32. The method of Embodiment 31, wherein the data encodable nucleic acid polymer further comprises a first plurality of sets of spacer residues, each spacer residue linked via the nucleic acid polymer backbone, wherein each set of the first plurality comprises two or more spacer residues, wherein each set of the first plurality is provided in-between each pair of the plurality of pairs of convertible nucleobases to provide the iterative spacing among the plurality of pairs of convertible nucleobases.
Embodiment 33. The method of Embodiment 31 or 32, wherein the iterative spacing among the pairs of convertible nucleobases is equal to or greater than the resolution of the data encoding device.
Embodiment 34. The method of Embodiment 30, wherein the data encoding device comprises a plasmonic well or channel, and the method further comprising: transfer the data encodable nucleic acid polymer into the plasmonic well or channel of the data encoding device, wherein the plasmonic well or channel provides the light energy or redox energy to release the leaving group from the nucleobase structure of the at least one nucleobase.
Embodiment 35. The method of Embodiment 30, wherein the data encoding device comprises a STED laser system, and the method further comprising: stretching the data encodable nucleic acid polymer and focusing the STED laser onto the stretched data encodable nucleic acid polymer, wherein the STED laser provides the light energy or redox energy to release the leaving group from the nucleobase structure of the at least one nucleobase.
Embodiment 36. A method of encoding data onto a data encodable nucleic acid polymer, comprising:
providing a data encodable nucleic acid polymer that comprises:
   a first plurality convertible nucleobases stochastically or irregularly spaced along the nucleic acid polymer and linked via the nucleic acid polymer backbone, wherein each convertible nucleobase of the first plurality comprises a first nucleobase structure and a first leaving group, the first leaving group linked to the first nucleobase structure via a first linker, and wherein each convertible nucleobase of the first plurality is provided in a first state and is capable of being converted from the first state into a second state by light energy or redox energy that releases the first leaving group from the first nucleobase structure; and
   a second plurality of convertible nucleobases stochastically or irregularly spaced along the nucleic acid polymer and linked via the nucleic acid polymer backbone, wherein each convertible nucleobase of the second plurality comprises a second nucleobase structure and a second leaving group, the second leaving group linked to the second nucleobase structure via a second linker, and wherein each convertible nucleobase of the first plurality is provided in a first state and is capable of being converted from the first state into a second state by light energy or redox energy that releases the second leaving group from the second nucleobase structure; and
selectively converting, utilizing a data encoding device, a subset of the convertible nucleobases of the first plurality and the second plurality into the second state by providing a light energy or redox energy to release the leaving group from the nucleobase structure of the convertible nucleobases.
Embodiment 37. The method of Embodiment 36, wherein the subset of the convertible nucleobases of the first plurality and the second plurality that are selectively converted are based on a data code to be encoded.
Embodiment 38. The method of Embodiment 37, wherein the selective conversion of nucleobases yields a nucleic acid polymer comprising convertible nucleobases in between converted nucleobases.
Embodiment 39. The method of Embodiment 36, wherein the data encoding device comprises a plasmonic nanopore, and the method further comprising:
passing the data encodable nucleic acid polymer through the plasmonic nanopore of the data encoding device, wherein the plasmonic nanopore provides the light energy or redox energy to release the leaving group from the nucleobase structure of the convertible nucleobases.
Embodiment 40. The method of Embodiment 30, wherein the data encoding device comprises a plasmonic well or channel, and the method further comprising:

transfer the data encodable nucleic acid polymer into the plasmonic well or channel of the data encoding device, wherein the plasmonic well or channel provides the light energy or redox energy to release the leaving group from the nucleobase structure of the convertible nucleobases.
Embodiment 41. The method of Embodiment 30, wherein the data encoding device comprises a STED laser system, and the method further comprising:
stretching the data encodable nucleic acid polymer and focusing the STED laser energy onto the stretched data encodable nucleic acid polymer, wherein the STED laser provides the light energy or redox energy to release the leaving group from the nucleobase structure of the convertible nucleobases.
Embodiment 42. A method for decoding data from a data encoded nucleic acid polymer, the method comprising:
providing a plurality of redundant copies of a data encoded nucleic acid polymer that comprises:
   a plurality of converted nucleobases, wherein each converted nucleobase comprises first nucleobase structure, wherein the first converted nucleobase has been converted from a first state into a second state by light energy or redox energy that released a first leaving group from the first nucleobase structure; and
   a plurality of convertible nucleobases, wherein each convertible nucleobase comprises a nucleobase structure and a leaving group, the leaving group linked to the second nucleobase structure via a linker, and wherein the convertible nucleobase is provided in a first state and is capable of being converted from the first state into a second state by light energy or redox energy that releases the second leaving group from the second nucleobase structure
   wherein the converted nucleobases and convertible nucleobases are linked via the nucleic acid polymer backbone; and
sequencing each redundant copy of the plurality of redundant copies;
detecting the plurality converted nucleobases and the plurality of convertible nucleobases;
decoding the data based on the detected plurality of converted nucleobases.
Embodiment 43. The method of Embodiment 42, wherein the plurality of the plurality converted nucleobases and the plurality of convertible nucleobases are detected based on the sequencing result of the redundant copies of the data encoded nucleic acid polymer.
Embodiment 44. The method of Embodiment 43, wherein a sequencing result indicating a mix of nucleobase structures at a particular nucleobase indicates a convertible nucleobase that is not a part of the data code.
Embodiment 45. A nucleic acid polymer for encoding data, comprising:
a first plurality convertible nucleobases regularly or irregularly spaced along the nucleic acid polymer and linked via the nucleic acid polymer backbone, wherein each convertible nucleobase of the first plurality comprises a first nucleobase structure and a first leaving group, the first leaving group linked to the first nucleobase structure via a first linker, and wherein each convertible nucleobase of the first plurality is provided in a first state and is capable of being converted from the first state into a second state by light energy or redox energy that releases the first leaving group from the first nucleobase structure; and
a second plurality of convertible nucleobases regularly or irregularly spaced along the nucleic acid polymer and linked via the nucleic acid polymer backbone, wherein each convertible nucleobase of the second plurality comprises a second nucleobase structure and a second leaving group, the second leaving group linked to the second nucleobase structure via a second linker, and wherein each convertible nucleobase of the first plurality is provided in a first state and is capable of being converted from the first state into a second state by light energy or redox energy that releases the second leaving group from the second nucleobase structure.
Embodiment 46. The nucleic acid polymer of Embodiment 45 further comprising a plurality of spacer residues linked via the nucleic acid polymer backbone, wherein spacer residues are provided in between the convertible nucleobases.
Embodiment 47. A data encoded nucleic acid polymer, comprising:
a first plurality of converted nucleobases regularly or irregularly spaced along the nucleic acid polymer and linked via a nucleic acid polymer backbone, wherein each converted nucleobase of the first plurality comprises a first nucleobase structure, wherein each converted nucleobase of the first plurality has been converted from a first state into a second state by light energy or redox energy that released a first leaving group from the first nucleobase structure; and
a second plurality of converted nucleobases regularly or irregularly spaced along the nucleic acid polymer and linked via a nucleic acid polymer backbone, wherein each converted nucleobase of the second plurality comprises a second nucleobase structure, wherein each converted nucleobase of the second plurality has been converted from a first state into a second state by light energy or redox energy that released a second leaving group from the second nucleobase structure.
Embodiment 48. The data encoded nucleic acid polymer of Embodiment 47, further comprising:
a first plurality of convertible nucleobases regularly or irregularly spaced along the nucleic acid polymer and linked via the nucleic acid polymer backbone, wherein each convertible nucleobase of the first plurality comprises the first nucleobase structure and the first leaving group, wherein the first leaving group is linked to the first nucleobase structure via a first linker; and
a second plurality of convertible nucleobases regularly or irregularly spaced along the nucleic acid polymer and linked via the nucleic acid polymer backbone, wherein each convertible nucleobase of the second plurality comprises the second nucleobase structure and the second leaving group, wherein the second leaving group is linked to the second nucleobase structure via a second linker.
Embodiment 49. The nucleic acid polymer of Embodiment 48 further comprising a plurality of spacer residues linked via the nucleic acid polymer backbone, wherein spacer residues are provided in between nucleobases comprising the converted and convertible nucleobases.

### Exemplary Embodiments

Described herein are various examples of compositions, systems, and methods for data storage utilizing nucleic acid polymers. Examples of writable nucleic acid polymers, methods to produce such polymers, methods to writing data, and methods for reading data are provided.

### Example 1: Writable DNA Polymer with MeNPOC Nucleobases

A writable nucleic acid molecule can be generated to comprise bits, data fields, spacers, delimiters, and/or a terminal identifier tag. In this example, a converted nucleobase (i.e., “1”) is 5-aminopropynyl-deoxyuridine, and an unconverted nucleobase (i.e., “0”) is the same molecule with the amine group substituted by a MeNPOC group, which can be efficiently removed by light (see P. Klan, et al., Chem Rev. 2013; 113:119-91, the disclosure of which is incorporated herein by reference). The writable nucleic acid is constructed with all convertible nucleobases having an MeNPOC-substituted deoxyuridine base, which is denoted “0” in the following example:

Data field: 5′-C-(A)₆-0-(A)₆-0-(A)₆-0-(A)₆-0-(A)₆-0-(A)₆-0-(A)₆-0-(A)₆-0-(A)₆-(C)-3′

The data field contains “0” bits spaced by six adenine nucleotides (A) to allow for spatial resolution for writing via focused light energy. It is shown here with eight bits (one “byte” in 8-bit architecture). The cytosines at the ends can provide a data delimiter function, signifying a break between one 8-bit field and the next. It is understood that spacers and delimiter are not limited to adenosines and cytidines and could be almost any single or multiple natural or unnatural residue that is detectably different from the convertible nucleobases and, preferably, is unreactive to the writing mechanism. It is also understood that a delimiter may not be needed to achieve efficient data encoding. In such a case, a writable nucleic acid contains repeating bits and spacers that are not contained within delimiters. It is also understood that the spacing and number of spacers between bits can be readily altered to reflect the resolution and precision of the writing method.

The writable nucleic acid polymer consists of the data field sequence repeated in a string. The polymer can be tagged at the 5′ or 3′ end by a data tag. This can comprise a sequence of natural bases that denote time, date, type of data, user, or other useful identifying information. It is understood that a data tag may not be necessary for some applications, as identifying information can be written directly into the data fields.

### Example 2: Writable Nucleic Acid Polymer Produced by Rolling Circle Reaction

In this example, a circular DNA oligonucleotide encoding the repeating “data field” in example 1 as described. The circle is chosen to be complementary to the repeating unit, and is chosen in this case to be 57 nucleotides in size, which falls in a size range that is known to act as a good substrate for DNA polymerase-mediated rolling circle synthesis (see M. G. Mohsen and E. T. Kool, Acc Chem Res. 2016 Nov. 15; 49(11): 2540-2550, the disclosure of which is incorporated herein by reference). The circle sequence is as follows: 5′-GTTTTTTATTTTTTATTTTTTATTTTTTATTTTTTATTTTTTATTTTTTATTTTTTG-3′ where the 5′ and 3′ ends are joined intramolecularly to make a circle.

A DNA primer is constructed with a 3′ end complementary to the circle. An example of an effective primer sequence is below:

Primer:
5′-IDsequence-AAAAAATAAAAAACCAAAAAA-3′

The ID sequence is optional. The DNA primer is annealed to the DNA circle in a Mg²⁺-containing buffer that supports DNA polymerase activity. The mixture is contacted with nucleoside triphosphates (dNTPs) that will comprise the repeating data field. For the data field in example 1, the necessary dNTPs are 5-nitroveratryl-oxycarbonyl-aminoproynyl deoxyuridine 5′-triphosphate, dATP, and dCTP. Contacting this solution with a suitable DNA polymerase enzyme at a temperature supportive of enzyme activity produces a long repeating writable DNA polymer, comprising repeating data fields, and a DNA data identifier tag at the 5′ end. Gel analysis shows that the blank tape is 10,000 to 50,000 nucleotides in length. It is isolated from the smaller polymerase, nucleotides, and circle by size exclusion chromatography, column purification, precipitation, gel electrophoresis, or by other purification methods, and is stored in the dark to avoid stray bit writing.

Various DNA polymerase enzymes for rolling circle synthesis have been described (see S. Ishino and Y. Ishino, Front Microbiol. 2014; 5:465, the disclosure of which is incorporated herein by reference). Examples include phi29 and BST3.0 polymerases. A polymerase with high processivity enables longer writable DNA polymers to be produced. A polymerase with the ability to efficiently accept modified nucleotides (such as the modified deoxyuridine described here) as substrates can be used.

### Example 3: Writable Nucleic Acid Polymer Produced by Synthesis and Ligation

In this example, a ligase enzyme is used to assemble single-stranded and/or double-stranded writable DNA polymers containing the convertible nucleobase O6-ortho-nitrobenzylG (see FIG. 3D, denoted X here), which is not efficiently incorporated into DNA by most polymerase enzymes due to its blocked base pairing ability. The designed 8-bit repeating data field sequence is the following:

5′-CCT-(A)6-X-(A)6-X-(A)6-X-(A)6-X-(A)6-X-(A)6-X-
(A)6-X-(A)6-X-(A)6-CGA-3′

A ligatable oligonucleotide comprising the single 8-bit field is synthesized with the following sequence:

5′-pCCT-(A)6-X-(A)6-X-(A)6-X-(A)6-X-(A)6-X-(A)6-
X-(A)6-X-(A)6-X-(A)6-(CGA)-3′

Where “p” denotes a terminal phosphate group. A splint for ligating this sequence is synthesized with the following sequence:

5′-TTTTTTAGGTCGTTTTTT-3′

Contacting this splint and the data field oligonucleotide with T4 DNA ligase and ATP in a ligase-supporting buffer results in joining of many data field oligomers end-to-end, resulting in a long polymer strand. Gel analysis of this product reveals a ladder of lengths ranging from 5000-50,000 nucleotides in size. If desired, portions of the “data field” DNA product can be split up and ligated at one end separately with different DNA identifiers, to be used separately in data writing. The long data fields are used for writing as a mixture of lengths. Alternatively, use of an electrophoresis gel and cutting out and eluting a specific band results in a blank tape DNA of homogeneous length.

A double-stranded writable DNA polymer is obtained by similar methods. In this case, the first data field oligonucleotide is also employed, but a different complement is used in the formation of a duplex with sticky ends. The sequence of this complementary oligonucleotide is as follows:

5′-
pGTTTTTTCTTTTTTCTTTTTTCTTTTTTCTTTTTTCTTTTTTCTTTTT
TCTTTTTTCTTTTTTAGGTC-3′

Hybridization of the complementary oligonucleotide with the data field oligonucleotide results in a duplex with sticky ends. Ligation with T4 DNA ligase and ATP results in a long repeating DNA double-stranded polymer. Gel analysis of this product reveals a ladder of lengths ranging from 5000-50,000 base pairs in size. If desired, portions of the data field DNA product can be split up and ligated at one end separately with different DNA identifiers, to be used separately in data writing. The long data fields are used for writing as a mixture of lengths. Alternatively, use of an electrophoresis gel and cutting out and eluting a specific band results in a blank tape DNA of homogeneous length.

### Example 4: Data Writing Via Light

A nanopore device with a plasmonic bow tie on the exit side of the pore is used to write digital data on the writable DNA polymer from example 1. Nanopores with plasmonic bow ties have been described (see X. Shi, et al., Small. 2018 May; 14(18):e1703307, the disclosure of which is incorporated herein by reference). The writable polymer is dissolved in an electrolyte solution and is moved through the pore at a regular rate via applied potential across the two sides of the pore. The test bit sequence “01100101” is written repeatedly. This is achieved by flashing a beam of light on the nanoplasmonic structure at spaced time intervals to coincide with the bit spacing in a data field. Subsequent analysis by nanopore sequencing then reveals the sequence of “1” and “0” bits, and the repetition allows the analysis of the precision and errors in bit writing. Statistical analysis and data correction on the repeat units in the sequence confirms the intended bit sequence. Subsequent experiments with longer data strings reveal the ability to encode more data per molecule. Comparison of multiple copies of DNA tapes written with the same data enables sequence comparison and error correction.

### Example 5: Writing Data Via DNA Stretching and Light

In this example, data is encoded in the double-stranded writable DNA polymer from Example 3 by DNA stretching or combing, combined with local illumination to write bits. In the stretching/combing technique, flow is used to stretch individual DNA molecules with lengths of tens of thousands of nucleotides on a slide or other solid support, and the locations of the long DNAs are visualized by simple dyes added to solution (see T. F. Chan, et al., Nucleic Acids Res. 2006; 34:e113; and S Takahashi, M. Oshige, and S. Katsura, Molecules. 2021; 26:1050; the disclosures of which are each incorporated herein by reference). Light is focused progressively along the strand at intended “1” sites along the strand to convert nucleobase bits from the “0” state to the “1” state. The light illumination is achieved at high resolution by the use of the STED technique, which uses two lasers to illuminate locally with high precision (see G. Vicidomini, P. Bianchini, and A. Diaspro, Nat Methods. 201; 15:173-182, the disclosure of which is incorporated herein by reference).

The resulting written DNAs can be stored for archiving. When the data is to be retrieved, the stored data can be read by nanopore sequencing of the DNA polymer (see Example 7).

In another embodiment, the bit nucleotide comprises a fluorescent dye linked by a photocleavable linker to a fluorescence quencher. The presence of the quencher keeps the unwritten DNA nonfluorescent. “Localized illumination” of the “stretched DNA” strand results in cleavage of the linker, resulting in loss of the quencher, rendering the local nucleotide fluorescent. Progression of the photoexciting light along the stretched data field DNA results in writing bits at data-encoding intervals. The slide is stored as written data. When the data is to be retrieved, it is read by imaging the strand on the slide and analyzing the “1” bits as fluorescent spots; the spacing denotes the presence and numbers of intervening “0” bits.

### Example 6: Writing Data Via Redox

This example describes the writing of data by redox with writable DNA polymers comprising the redox-reactive nucleotide in FIG. 3G. In this experiment, a nanopore device with an electrode at the pore is employed. A DNA blank tape containing redox-reactive nucleobases is passed through the pore at a controlled rate. As the DNA passes through, reductive voltage potential is applied as a pulse at timed spacing. This results in reduction and loss of the group on the “0” bit, switching it to the aminopropyne group which encodes “1”. Spacing in time of applied reduction results in variable but predictable spacing of “1” and “0” groups, which defines the digital data.

### Example 7: Reading Written DNA Polymers Via Nanopore Sequencing

Common nanopore sequencing devices measure current flow of electrolytes during passage of a DNA molecule through the pore. Since DNA bases each differ in size and shape, this slightly alters the current as each different base passes the pore. In this example, an experiment is carried out with a commercial nanopore device, and the readout changes in current over time while a written DNA tape passes through. In this case, the single-stranded written DNA polymer produced in Example 3 and written as in Example 4 is employed. The “1” and “0” bits comprise G and nitrobenzylG, which differ considerably in size. Experiments with DNA tapes having bits in all-“0” state (blank polymer) reveal the lowering of current when the largest nitrobenzylG nucleotides pass through, and can distinguish the differences in current between these “0” bits and the spacers and delimiters. Separately, DNA all-“1” polymers are measured, showing the level of current observed as the “1” (G) bits pass though. These experiments provide calibration for reading and distinguishing current levels that denote “1” and “0” bits. Next, fully written DNA polymers are passed though. Current levels denoting “1” and “0” are read and placed in context of current levels seen for spacers and delimiters. Multiple reads of the same strand are used, if needed, to improve accuracy of data reading.

### Example 8. Dual Bit Writable Nucleic Acid Polymers

This example provides a writable nucleic acid polymer design that enables the writing of both “1” and “0” bits with an active signal. In this design, zeros are not passively included in the data field, but rather require an active switching signal. Photo removable groups can be triggered at distinct wavelengths of light. FIGS. 13A-13C show examples of nucleotides comprising a group that can be removed by irradiation at 325 nm, and a different group that can be removed at 400 nm irradiation. If these two groups are placed near one another in a data field of the blank DNA tape, a light pulse at 400 nm removes only one of the two groups in the pair. On the other hand, a light pulse at 325 nm results in loss of both of the groups. These two outcomes are akin to “0” and “1” for encoding data.

### Example 9: Construction of Data Encodable DNA

A 141 nt DNA strand is synthesized to contain pairs of iteratively repeating convertible nucleobases (X and Y) separated by two spacer nucleobases, with each pair representing a bit of encodable data. Each pair of nucleobases is separated with ten intervening spacer nucleobases. The total number of pairs in the strand is 11, and thus the DNA can encode 11 bits of “one” and “zero” data. The sequence of this 150mer is:

5′-
TCGATTXAYAATTATTCCTXAYAATTATTCCTXAYAATTATTCCTXAYA
ATTATTCCTXAYAATTATTCCTXAYAATTATTCCTXAYAATTATTCCTX
AYAATTATTCCTXAYAATTATTCCTXAYTTTATCTTATXAYTCG
A-3′ = 141

where X denotes O6-nitrobenzylguanine and Y denotes N6-coumarinylmethyl-adenine.

A complementary DNA sequence is synthesized to be complementary to the first strand such that a duplex can be formed. The complementary sequence can be designed to create overhanging sticky ends, and the two strands are further modified with 5′ phosphate groups. The sequence of this 141mer is:

5′-
TCGATTCATAAGATAAATTCAGGAATAATTTTCAGGAATAATTTTCAGG
AATAATTTTCAGGAATAATTTTCAGGAATAATTTTCAGGAATAATTTTC
AGGAATAATTTTCAGGAATAATTTTCAGGAATAATTTTCAATCGA-3′

Note that the bases in this complement are designed to be complementary to the converted versions of bases X and Y. Longer DNAs can store more data per molecule. To generate longer nucleic acid polymers for data storage, the two DNA strands can be mixed in a Mg2+-containing buffer that supports hybridization and enzymatic ligation. ATP and T4 DNA ligase are added, resulting in end-to-end joining of the 150 nt DNAs into longer polymer chains, having lengths of ˜300 bp and more, including DNAs of ˜1500 bp as analyzed by agarose gel electrophoresis. Data encodable DNAs of preferred size can be isolated by gel electrophoresis and extracted. Accordingly, data encodable polymers can be provided and utilized as a mixture of lengths or having specific lengths by excising specific bands.

### Example 10: Data Encoding into a Polymer

A nanopore device with a plasmonic bow tie on the exit side of the pore is used to write digital data on the data encodable DNA polymer from Example 9. Nanopores with plasmonic bowties have been described (see X. Shi, et al., Small. 2018 May; 14(18):e1703307, the disclosure of which is incorporated herein by reference). The data encodable polymer is dissolved in an electrolyte solution and is moved through the pore at a regular rate via applied potential across the two sides of the pore. The data sequence “01100101100” is encoded in the polymer (for the first 150 nucleotides). This is achieved by flashing a beam of light on the nanoplasmonic structure at spaced time intervals to coincide with the paired bit spacing.

To encode a bit of data, light energy can be provided by 400 nm wavelength onto the bit pair to release the coumarinylmethyl group from the N6-coumarinylmethyl-adenine to convert the nucleobase into an adenine. The light energy at 400 nm does not affect the O6-nitrobenzylguanine, leaving the nucleobase unconverted. This bit pair conversion can be denoted a “zero.” Likewise, light energy can be provided by 365 nm wavelength onto the bit pair to release the nitrobenzyl group from the O6-nitrobenzylguanine to convert the nucleobase into a guanine and to release the coumarinylmethyl group from the N6-coumarinylmethyl-adenine to convert the nucleobase into an adenine. This bit pair conversion can be denoted a “one.” Data encoding can continue to yield the data sequence “01100101100,” which structurally would have the following nucleobase sequence:

5′-
TCGATTXAAAATTATTCCTGAAAATTATTCCTGAAAATTATTCCTXAAA
ATTATTCCTXAAAATTATTCCTGAAAATTATTCCTXAAAATTATTCCTG
AAAATTATTCCTGAAAATTATTCCTXAATTTATCTTATXAATCGA-3′

where X denotes O6-nitrobenzylguanine and Y denotes N6-coumarinylmethyl-adenine. Notably, multiple copies can be encoded such that decoding can be performed by SBS as unconverted nucleobases will be read as a mixture of bases in the sequencing result.

### Example 11: Decoding Data from Encoded DNA

After data has been encoded into a 1500 bp DNA strand by use of a nanopore device combined by use of dual wavelength light pulses, the resulting DNA is ready for decoding (“reading”) when the data is to be recovered. The DNA can be encoded with a multiplicity of approximately 10 to 100 copies, the encoded DNA contains enough copies to enable mixtures of outcomes to be decoded. The DNA is sequenced by use of long-read single-molecule sequencing by synthesis (Pacific Biosciences). The sequence output shows that the convertible bases are sequenced as expected, with near 100% fidelity; 98% or better) reading as the bases that were in the original assembly. Where a “zero” is encoded, the coumarinyl group is removed from the N6-coumarinylmethyl-adenine, resulting in formation of adenine. Thus, the signal of “A” is found to be enhanced over that of N6-coumarinylmethyl-adenine at this position. However, the O6-nitrobenzylguanine sequencing signature in the same bit pair reads as mix of G and A. At positions encoded to be “one”, both the coumarinyl group and the nitrobenzyl groups are removed, resulting in both the A signal being enhanced at position Y in the bit and adenine signal being enhanced at position X the same bit pair.

### Example 12: Stochastic or Irregular Data Encoding

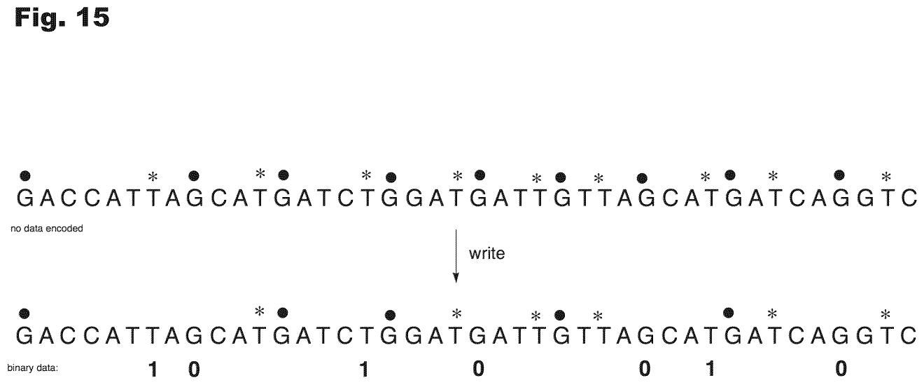

In this example, the convertible nucleobases are provided irregularly spaced along the polymer. The data encodable polymer comprises O6-nitrobenzylguanine and O4-nitrobenzylthymine along the strand. Conversion of O6-nitrobenzylguanine into guanine can be denoted as a “zero” and conversion O4-nitrobenzylthymine into thymine can be denoted as a “one.” As the polymer pass through the nanopore, data is encoded by selectively converting the appropriate convertible nucleobase in accordance with a data code. Furthermore, convertible nucleobases can be skipped to ensure the correct code is encoded. FIG. 15 illustrates a DNA polymer before and after data encoding, in which a code of “1010010” is encoded. Several convertible nucleobases are skipped and left unconverted in the process. When the encoded data is decoded, only the converted nucleobases are utilized to decipher the data code and the unconverted bases are ignored. When using SBS, multiple redundant encoded DNA polymers can be utilized to decipher whether a particular nucleobase is unconverted (e.g., by providing reads of mixed nucleobase structures) or converted (e.g., by providing reads a singular nucleobase structure).

### Example 13: Constructing “Writeable” DNA with Modified Convertible Nucleobases at Regular Intervals

The convertible base O6-coumarinylG (G*) is synthesized as a deoxynucleoside triphosphate derivative (dG*TP). It acts as a polymerase substrate when a DNA template is provided to contain a complementary base, such as “benzi” (see, e.g., C. M. N. Aloisi et al., J. Am. Chem. Soc 2020, 142(15):6962-6969). Benzi is known to pair selectively with O6alkylG modified bases.

A circular single-stranded DNA oligonucleotide is constructed having 60 nucleotides in size, with a single “benzi” nucleotide in the sequence. The other 59 nucleotides comprise native A, C, T, and G nucleotides. A DNA primer (20 nt in length) (1 μM) complementary to a non-benzi region of the circle is added to a solution of the circle (1 μM) in polymerase-supporting buffer. To induce a “rolling circle” DNA synthesis, Phi29 polymerase is added along with five nucleotides at 500 uM each (dATP, dGTP, dCTP, dTTP, and dG*TP), under suitable conditions known for the Phi29 polymerase activity. After 4 hours, the resulting solution has long repeating single-stranded DNAs of varying length but many over 10 kB in length as judged by agarose gel electrophoresis with size markers. Sequencing of the single-stranded DNAs in the solution confirms that the repeating sequence contains a G* base once per repeat, evenly spaced at 60 nucleotides apart.

This solution of single-stranded DNAs is converted to double-stranded form by using a primer complementary to this repeating sequence, along with four native nucleoside triphosphates and phi29 polymerase. The result is a solution of long double-stranded DNAs containing single G* modified bases every 60 bp.

This polymerase approach, together with modified DNA bases, is used to solve the problem of incorporating photomodifiable groups into nucleobases in a DNA where the photomodifiable groups are not substrates for polymerase enzymes.

To construct a repeating DNAs containing a second modified base, a modification of this strategy is used. The modified base T* is synthesized as a deoxynucleoside triphosphate derivative. T* is O4-nitrophenethylT, containing a group NPE that can be removed with light. O4-alkylT is known to pair with polymerases opposite G. See, e.g., M. K. Dosanjh et al., Carcinogenesis 1993, 14(9):1915-1919.

A second circular DNA containing benzi is constructed once in the sequence. In this case, there is also only one C in the sequence, placed 10 nt away from benzi; the remainder of the bases are G, C, and T. Using DNA polymerase and primer as described above, together with the same five nucleotides above (dATP, dGTP, dCTP, dTTP, and dG*TP) produces long repeating DNAs containing G* once per repeat and a single G per repeat ten nucleotides away. Use of a DNA primer complementary to this repeat, combined with polymerase and nucleotides (dTTP, dGTP, dATP, dT*TP, with no dCTP) results in synthesis of long repeating DNA duplexes containing G* once per repeat and T* once per repeat, ten bp away from G* and in the opposite strand.

This example shows that writable DNAs with photo-removable nucleobases at regular intervals can be synthesized using nucleotide with photo-removable nucleobases (e.g., photo-removable nucleobases that will convert to natural nucleobase after conversion by light) in the presence of polymerase. This method can utilize polymerase for controllable production of longer strands of DNAs. DNAs produced using this method are significantly longer than those DNAs can only be synthesized by ligations of synthetic oligos, such as DNAs with backbone modifications.

### Example 14: Writing “Scarless” Data in DNA and Reading with Long-Read SMRT Sequencing

A 20 kb DNA is constructed to contain two modified convertible nucleobases (X and Y) that can be converted to native DNA nucleobases upon “writing” by photoirradiation. The positions of all modifications are known, and are repeatedly spaced with distance of about 60 base pairs (ca. 20 nm) between each occurrence of a given modification. That is, X is located approximately 60 base pairs (bp) from the adjacent X, and Y approximately 60 bp from adjacent Y. Both modifications (X and Y) are within 10 base pairs of each other, such that a given pair or duad of X/Y is simultaneously exposed in a given localized photoexcitation event. This DNA assembly is denoted “DNA blank tape”. Mixed polymerases can be used for incorporation of two or more modified nucleobases in the DNA blank tape.

Nucleobase X is guanine modified with an O-nitrophenethyl (NPE) group directly attached without linker or sidechain at 0-6. It can be converted to native guanine (i.e., without a scar) by irradiation at 360 nm. In this example, the 0-6 modified guanine is the “unwritten” (“blank”) form of the nucleotide, and after successful removal by irradiation, the guanine product is considered written, and its interpretation as 1 or 0 depends on the state of a nearby Y modification.

Previous work has shown that guanine modified by an alkyl group at 0-6 can be read by a polymerase enzyme via sequencing by synthesis. See, e.g., A. M. Kietrys, J. Am. Chem. Soc. 2017, 139(47); 17074-17081. It typically codes for a mixture of A and G among the numerous reads of the sequence. The quantitative percentages of coding depend on which exact modification and which polymerase is used to read it, and this is measured beforehand (in a calibration experiment) by SMRT sequencing of synthetic DNA fragments containing the modification. Consensus reads yield the percentages of base encoding for this modification. For example, one can observe that on rereading the same DNA fragment, one sees that the polymerase inserts C opposite the modified base in 30% of reads (interpreting the base as “G”), and inserts T opposite the base in 64% of reads (interpreting it as “A”). This mixed signal for a single modified base is a signal (a fingerprint) of an unwritten bit. If the base in that single molecule is successfully photoconverted to G, then essentially 100% of reads will interpret it as G.

If there are multiple copies (for example, 1000 copies) of the same DNA molecule containing this modification at one position, and the DNA is irradiated by light in bulk solution at 360 nm to the extent that the NPE group is removed in 50% of the DNAs, then this change remains readable by sequencing by synthesis. Its consensus read is a 50% average between the fingerprint of the modified nucleobase (i.e., O-6 nitrophenethyl substituted guanine) and that of the native nucleobase (i.e., guanine). Thus the user can read data that is encoded by light at less than 100% complete yield.

Also in this example, nucleobase Y is thymine modified with a coumarinyl (Coum) group at O-4. It can be converted to native thymine in a “scarless reaction” by photoirradiation at 360 nm or 400 nm. Similar to the analysis of guanine above, a calibration is done with SMRT sequencing to determine its mixed coding percentages, distinct form that of native thymine. This mixed coding percentage is a fingerprint denoting an unconverted Coum-thymine, such as occurs in an unwritten bit. When Coum-thymine is photoconverted to a native nucleobase thymine (T), it codes as native T, essentially 100% of reads. As for nucleobase X, one can interpret partial conversion among multiple copies of DNA by observing an averaging of the fingerprints of the modified nucleobase Y and native nucleobase T.

In this example, a “0” bit is interpreted as such when T-Coum in a G-NPE/T-Coum pair is converted to T via irradiation at 400 nm. If both modifications are removed (using 360 nm irradiation), the bit is interpreted as “1”. Again, reads of multiple copies of the data can be used to interpret bits that are converted below the 100% maximal yield.

Writing a data “bit” locally makes use of local irradiation or local excitation method such as translocating a STED microscope irradiation beam along the DNA, or translocating the DNA in a zero mode waveguide or through a plasmonic nanopore using methods known in the art.

Note that the blank tape DNA in this example is modified with approximately evenly spaced X and Y everywhere in the DNA sequence. Thus it contains the potential to be written with binary data anywhere. Pairs of X,Y modified groups are simply regarded as lacking data (i.e., unwritten). The identical data can be written starting anywhere in the DNA (assuming there is enough length to complete the writing process). Since the DNA positioning relative to the writing light can stochastically vary, and the speed of translocation can vary, one can still write and read data by interpreting the string of 0 and 1 bits, skipping over “blank” bits. This has the advantage of not requiring careful positioning of the start and stop site of writing, and does not require perfect control over translocation speed. Because there is no need to pause to position bits, the writing method is simpler and faster than methods that function by controlling the translocation and exact position of the DNA polymer through a nanopore.

Data encoding the letter “e” are written into the DNA blank tape at the single molecule level using a superresolution microscope on stretched DNA molecules on a slide. The 8-bit Unicode binary string for letter “e” is 01100101, using eight pulses of 360 nm light (1) and/or 400 nm light (0) from a superresolution microscope at 20 nm resolution. The writing is done 1000 times on 1000 single molecules, collecting the DNAs at the end by washing the slide containing them.

This “written” DNA is submitted to SMRT sequencing. Positions showing the fingerprints of modified nucleobases (as a G-NPE/T-Coum pair) are interpreted as blank and not encoding data. Paired bit positions in which the consensus of reads show an averaging of the fingerprints of modified and unmodified bases are interpreted as data; selectively unblocking of T by removing NPE indicates a “0” and paired bit positions that show substantial conversion of both T and G indicate a written bit of “1”. Progressing along the strand in order generates the bit string 01100101, indicating the storage of data (data conversion interprets it as the letter “e”).

Note that data correction can be optionally used to correct errors. For example, if most single molecule DNA copies yield a string of 01100101, but other binary strings are also present, comparisons of binary data can lead to the correct conclusion. For example, some missed bits may occur (example 0100101) or the data may run out because the end of the DNA can be reached (example 01100). However the comparison of these different strings leads to the correct conclusion even with these errors. This dual bit active writing enables the user to write more rapidly than would be possible if specific positioning of the DNA were required.

1-113. (canceled)

114. A method, comprising:
(a) providing a polymer, wherein said polymer comprises:
(i) a plurality of convertible residues, wherein each of said convertible residues is in a first state, and is capable of being converted into a second state by a stimulus; and
(ii) a plurality of spacer residues, wherein each of said convertible residues of said plurality of convertible residues is separated by 2-50 spacer residues of said plurality of spacer residues, wherein each of said spacer residues of said plurality of spacer residues is not capable of being converted into the second state by said stimulus; and
(b) encoding data into the polymer by converting at least a subset of convertible residues of the plurality of convertible residues from the first state into the second state using a data writing device, wherein said data writing device applies said stimulus.

115. The method of claim 114, wherein said plurality of convertible residues are covalently linked to said polymer.

116. The method of claim 114, wherein said plurality of spacer residues are covalently linked to the polymer.

117. The method of claim 114, wherein said polymer is a single-stranded nucleic acid.

118. The method of claim 114, wherein said polymer is a double-stranded nucleic acid.

119. The method of claim 114, wherein said plurality of convertible residues comprises at least 10 convertible residues.

120. The method of claim 114, wherein said plurality of convertible residues comprises at least 100 convertible residues.

121. The method of claim 114, wherein said plurality of convertible residues comprises at least 1,000 convertible residues.

122. The method of claim 114, wherein said second state comprises a naturally occurring nucleobase.

123. The method of claim 114, wherein each of said spacer residue of said plurality of spacer residues is the same spacer residue.

124. The method of claim 114, wherein said plurality of spacer residues comprises naturally occurring nucleobases.

125. The method of claim 114, wherein said plurality of spacer residues comprises ethylene glycol residues.

126. The method of claim 114, wherein each of said convertible residues of said plurality of convertible residues is separated by 6-50 spacer residues of said plurality of spacer residues.

127. The method of claim 114, wherein each of said convertible residues of said plurality of convertible residues is separated by 2-10 spacer residues of said plurality of spacer residues.

128. The method of claim 114, wherein a conversion of said first state into said second state is an irreversible conversion.

129. The method of claim 114, wherein said plurality of convertible residues comprises non-naturally occurring nucleobases.

130. The method of claim 114, wherein each of said convertible residues of said plurality of convertible residues comprises a chemically modifiable moiety.

131. The method of claim 130, wherein said chemically modifiable moiety comprises one or more photo-removable groups.

132. The method of claim 131, wherein said chemically modifiable moiety is a leaving group.

133. The method of claim 114, wherein a ratio of convertible residues of said plurality of convertible residues to spacer residues of said plurality of spacer residues is between 2 to 1000.

134. The method of claim 114, wherein a ratio of convertible residues of said plurality of convertible residues to spacer residues of said plurality of spacer residues is between 2 to 100.

135. The method of claim 114, wherein said data writing device comprises a nanopore.

136. The method of claim 135, wherein said nanopore comprises a plasmonic nanopore that provides light pulses or redox energy.

137. The method of claim 136, wherein said plasmonic nanopore provides light pulses and wherein said light pulses comprise a wavelength from 400-850 nm.

138. The method of claim 137, wherein said stimulus comprises light pulses.

139. The method of claim 114, wherein said stimulus comprises voltage pulses.

140. The method of claim 114, wherein said stimulus comprises an enzymatic agent.

141. The method of claim 114, wherein said stimulus comprises a redox agent.

142. The method of claim 114, further comprising after (b) reading said data encoded into said polymer.

143. The method of claim 114, further comprising after (b) storing said polymer without a stabilizer.
